(19) United States
(12) Patent Application Publication
(10) Pub. No.: US 2007/0208001 A1
(43) Pub. Date: Sep. 6, 2007

(54) Modulators of 11- beta hydroxyl steroid dehydrogenase type 1, pharmaceutical compositions thereof, and methods of using the same

(76) Inventors: Jincong Zhuo, Boothwyn, PA (US); Yun-Long Li, Wilmington, DE (US); Meizhong Xu, Hockessin, DE (US); Chunhong He, Boothwyn, PA (US); Wenqing Yao, Kennett Square, PA (US)

Correspondence Address: FISH & RICHARDSON PC, P.O. BOX 1022, MINNEAPOLIS, MN, 55440-1022, US

(21) Appl. No.: 11/712,871

(22) Filed: Mar. 1, 2007

Related U.S. Application Data
(60) Provisional application No. 60/778,682, filed on Mar. 3, 2006.
(60) Provisional application No. 60/808,769, filed on May 26, 2006.

(51) Int. Cl. A61K 31/551; A61K 31/4747

(52) U.S. Cl. 514/212.02; 514/278; 540/543; 546/15

(57) ABSTRACT

The present invention relates to inhibitors of 11-β hydroxyl steroid dehydrogenase type 1 and pharmaceutical compositions thereof. The compounds of the invention can be useful in the treatment of various diseases associated with expression or activity of 11-β hydroxyl steroid dehydrogenase type 1.

### CROSS REFERENCE TO RELATED APPLICATIONS

This application claims the benefit of U.S. Ser. Nos. 60/778,682, filed Mar. 3, 2006, and 60/808,769, filed May 26, 2006, the disclosures of which are incorporated herein by reference in their entireties.

### FIELD OF THE INVENTION

The present invention relates to modulators of 11-β hydroxyl steroid dehydrogenase type 1 (11βHSD1), compositions thereof, and methods of using the same.

### BACKGROUND OF THE INVENTION

Glucocorticoids are steroid hormones that have the ability to modulate a plethora of biological processes including development, neurobiology, inflammation, blood pressure, and metabolism. In humans, the primary endogenously produced glucocorticoid is cortisol. Two members of the nuclear hormone receptor superfamily, glucocorticoid receptor (GR) and mineralcorticoid receptor (MR), are the key mediators of cortisol function in vivo. These receptors possess the ability to directly modulate transcription via DNA-binding zinc finger domains and transcriptional activation domains. This functionality, however, is dependent on the receptor having first bound to ligand (cortisol); as such, these receptors are often referred to as ‘ligand-dependent transcription factors’.

Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, being driven by corticotropin releasing hormone (CRH) produced by the paraventricular nucleus of the hypothalamus. The HPA axis functions to maintain circulating cortisol concentrations within restricted limits, with forward drive at the diurnal maximum or during periods of stress being rapidly attenuated by a negative feedback loop resulting from the ability of cortisol to suppress ACTH production in the anterior pituitary and CRH production in the hypothalamus.

The importance of the HPA axis in controlling glucocorticoid excursions is evident from the fact that disruption of this homeostasis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Interestingly, the phenotype of Cushing's syndrome patients closely resembles that of Reaven's metabolic syndrome (also known as Syndrome X or insulin resistance syndrome) including visceral obesity, glucose intolerance, insulin resistance, hypertension, and hyperlipidemia (Reaven (1993) Ann. Rev. Med. 44: 121-131). Paradoxically, however, circulating glucocorticoid levels are typically normal in metabolic syndrome patients.

For decades, the major determinants of glucocorticoid action were believed to be limited to three primary factors: 1) circulating levels of glucocorticoid (driven primarily by the HPA axis), 2) protein binding of glucocorticoids in circulation (upward of 95%), and 3) intracellular receptor density inside target tissues. Recently, a fourth determinant of glucocorticoid function has been identified: tissue-specific pre-receptor metabolism. The enzymes 11-beta hydroxysteroid dehydrogenase type 1 (11βHSD1) and 11-beta hydroxysteroid dehydrogenase type 2 (11βHSD2) catalyze the interconversion of active cortisol (corticosterone in rodents) and inactive cortisone (11-dehydrocorticosterone in rodents). 11βHSD1 has been shown to be an NADPH-dependent reductase, catalyzing the activation of cortisol from inert cortisone (Low et al. (1994) J. Mol. Endocrin. 13: 167-174); conversely, 11βHSD2 is an NAD-dependent dehydrogenase, catalyzing the inactivation of cortisol to cortisone (Albiston et al. (1994) Mol. Cell. Endocrin. 105: R11-R17). The activity of these enzymes has profound consequences on glucocorticoid biology as evident by the fact that mutations in either gene cause human pathology. For example, 11βHSD2 is expressed in aldosterone-sensitive tissues such as the distal nephron, salivary gland, and colonic mucosa where its cortisol dehydrogenase activity serves to protect the intrinsically non-selective mineralcorticoid receptor from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralcorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1 and a co-localized NADPH-generating enzyme, hexose 6-phosphate dehydrogenase (H6PD), can result in cortisone reductase deficiency (also known as CRD; Draper et al. (2003) Nat. Genet. 34: 434-439). CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS).

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the later, as well as b) the ability of 11βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local 11βHSD1-mediated conversion of inert glucocorticoid to active glucocorticoid can have profound influences whole body insulin sensitivity.

Based on this data, it would be predicted that the loss of 11βHSD1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Further, adipose-tissue overexpression of the 11-beta dehydrogenase enzyme, 11bHSD2, which inactivates intracellular corticosterone to 11-dehydrocorticosterone, similarly attenuates weight gain on high fat diet, improves glucose tolerance, and heightens insulin sensitivity. Taken together, these transgenic mouse studies confirm a role for local reactivation of glucocorticoids in controlling hepatic and peripheral insulin sensitivity, and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Addtionally, it was recently reported that these selective inhibitors of 11βHSD1 can ameliorate severe hyperglycemia in genetically diabetic obese mice. Data using a structurally distinct series of compounds, the adamantyl triazoles (Hermanowski-Vosatka et al. (2005) J. Exp. Med. 202: 517-527), also indicates efficacy in rodent models of insulin resistance and diabetes, and further illustrates efficacy in a mouse model of atherosclerosis, perhaps suggesting local effects of corticosterone in the rodent vessel wall. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, hyperlipidemia, and/or atherosclerosis/coronary heart disease. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). In vivo pharmacology studies with multiple chemical scaffolds have confirmed the critical role for 11βHSD1 in regulating insulin resistance, glucose intolerance, dyslipidemia, hypertension, and atherosclerosis. Thus, inhibition of 11βHSD1 is predicted to have multiple beneficial effects in the liver, adipose, skeletal muscle, and heart, particularly related to alleviation of component(s) of the metabolic syndrome, obesity, and/or coronary heart disease.

B. Pancreatic Function

Glucocorticoids are known to inhibit the glucose-stimulated secretion of insulin from pancreatic beta-cells (Billaudel and Sutter (1979) Horm. Metab. Res. 11: 555-560). In both Cushing's syndrome and diabetic Zucker fa/fa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release and the potential for attenuating pancreatic beta-cell decompensation.

D. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11βHSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

E. Intra-Ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

F. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawa et al. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11βHSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

G. Bone Disease

Gluccorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, WO 2004/065351, and WO 2005/108359. Antagonists of 11βHSD1 have also been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, atherosclerosis, androgen excess (hirsutism, menstrual irregularity, hyperandrogenism) and polycystic ovary syndrome (PCOS), therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1. The compounds, compositions and methods therein help meet this and other needs.

### SUMMARY OF THE INVENTION

The present invention provides, inter alia, compounds of formula Ia, Ib, Ic, Id, Ie, If and Ig: 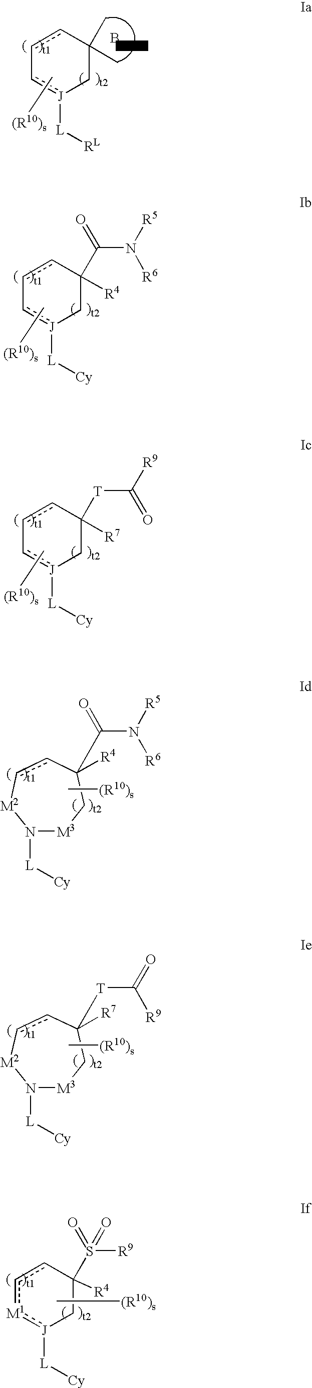 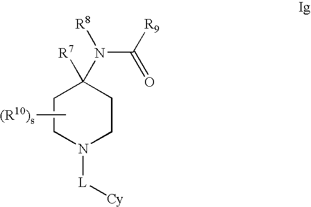
or pharmaceutically acceptable salts or prodrugs thereof, wherein constituent members are defined herein.

The present invention further provides methods of modulating 11βHSD1 by contacting 11βHSD1 with a compound of the invention.

The present invention further provides methods of inhibiting 11βHSD1 by contacting 11βHSD1 with a compound of the invention.

The present invention further provides methods of inhibiting the conversion of cortisone to cortisol in a cell by contacting the cell with a compound of the invention.

The present invention further provides methods of inhibiting the production of cortisol in a cell by contacting the cell with a compound of the invention.

The present invention further provides methods of treating diseases associated with activity or expression of 11βHSD1.

### DETAILED DESCRIPTION

The present invention provides, inter alia, a compound of of formula Ia, Ib, Ic, Id, Ie, If or Ig: 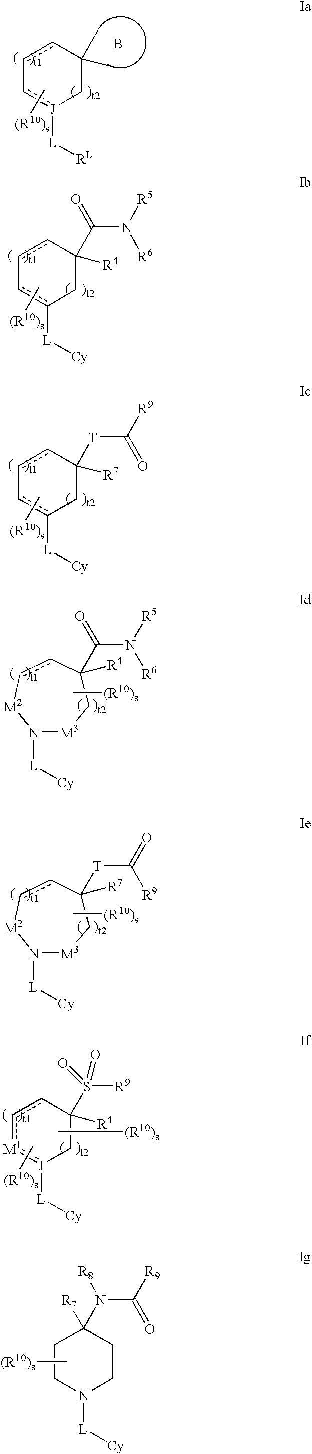
or a pharmaceutically acceptable salt or prodrug thereof, wherein:

Cy is aryl, heteroaryl, cycloalkyl, or heterocycloalkyl, each optionally substituted with 1, 2, 3, 4 or 5-W-X—Y-Z;

the ring-forming atom J is N or C;

L is absent, C₁₋₆alkenylenyl, (CR¹R²)_{q}, (CR¹R²)_{q1}O(CR¹R²)_{q2}, (CR¹R²)_{q1}S(CR¹R²)_{q2}, (CR¹R²)_{q1}SO₂(CR¹R²)_{q2}, (CR¹R²)_{q1}SO(CR¹R²)_{q2}, (CR¹R²)_{q1}SO₂NR³(CR¹R²)_{q2}, (CR¹R²)_{q1}COO(CR¹R²)_{q2}, (CR¹R²)_{q1}CO(CR¹R²)_{q2}, (CR¹R²)_{q1}NR^{3a}CONR³(CR¹R²)_{q2}, or (CR¹R²)_{q1}CONR³(CR¹R²)_{q2}, wherein the C₁₋₆ alkenylenyl is optionally substituted by 1, 2, 3, 4, 5 or 6 R^{1a};

M¹ is CH, CH₂, C(O), O, SO, SO₂, OC(O), NH, NHC(O), or NHSO₂;

M² and M³ are independently selected from absent, C(O), SO, SO₂, O, OC(O), NH, NHC(O), and NHSO₂, provided that at least one of M² and M³ is other than absent;

T is NR⁸, CH₂ or O;

ring B is a 3-14 membered cycloalkyl group or 3-14 membered heterocycloalkyl group which is optionally substituted by 1, 2, 3, 4 or 5-W^{a}-X^{a}-W′-X′—Y′-Z′;

[symbol] is a single or double bond;

R^{L} is Cy or C₁₋₆ alkyl wherein the C₁₋₆ alkyl is optionally substituted by 1, 2, 3, 4 or 5-W-X—Y-Z;

R¹ and R² are independently selected from H, halo, C₁₋₆ alkyl, C₁₋₆ haloalkyl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, S(O)R^{b′}, S(O)NR^{c′}R^{d′}, S(O)₂R^{b′}, and S(O)₂NR^{c′}R^{d′};

each R^{1a} is independently selected from halo, C₁₋₆ alkyl, C₁₋₆ haloalkyl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, S(O)R^{b′}, S(O)NR^{c′}R^{d′}, S(O)₂R^{b′}, and S(O)₂NR^{c′}R^{d′};

R³ and R^{3a} are independently selected from H, C₁₋₈ alkyl, arylalkyl, heteroarylalkyl, cycloalkyl, cycloalkylalkyl, heterocycloalkyl and heterocycloalkylalkyl, wherein each of the C₁₋₈ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′;

R⁴ is H, C(O)OR^{b′}, C(O)NR^{c′}R^{d′}, OR^{b′}, SR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹¹;

R⁵ is H or C₁₋₆ alkyl, wherein the C₁₋₆ alkyl is optionally substituted by 1, 2 or 3-W′-X′Y′-Z′;

or R⁴ and R⁵ together with the intervening —C—C(O)—N(R⁶)— moiety to which they are attached form a 4-14 membered heterocycloalkyl group which is optionally substituted by 1, 2 or 3-W′-X′—Y′-Z′;

R⁶ is C₁₋₆ alkyl, aryl, cycloalkyl, heteroaryl or heterocycloalkyl, each optionally substituted by 1, 2 or 3-W′-X′—Y′-Z′;

or R⁵ and R⁶ together with the N atom to which they are attached form a 3-14 membered heterocycloalkyl group which is optionally substituted by 1, 2 or 3-W′-X′—Y′-Z′;

R⁷ is H, C(O)OR^{b′}, C(O)NR^{c′}R^{d′}, OR^{b′}, SR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹¹;

R⁸ is H, C₁₋₆ alkyl, arylalkyl, heteroarylalkyl, cycloalkyl, cycloalkylalkyl, heterocycloalkyl or heterocycloalkylalkyl, wherein each of the C₁₋₆ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′;

or R⁷ and R⁸ together with the two adjacent atoms to which they are attached form a 3-14 membered heterocycloalkyl group which is optionally substituted by 1, 2 or 3-W′-X′—Y′-Z′;

R⁹ is H, C₁₋₆ alkyl, arylalkyl, heteroarylalkyl, cycloalkyl, cycloalkylalkyl, heterocycloalkyl or heterocycloalkylalkyl, wherein each of the C₁₋₆ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′;

or R⁸ and R⁹ together with the two adjacent atoms to which they are attached form a 3-14 membered heterocycloalkyl group which is optionally substituted by 1, 2 or 3-W′-X′—Y′-Z′;

or R⁷ and R⁹ together with the intervening —C-T-C(O)— moiety to which they are attached form a 4-14 membered heterocycloalkyl group which is optionally substituted by 1, 2 or 3-W′-X′—Y′-Z′;

or R⁴ and R⁹ together with the intervening —C—S(O)₂— moiety to which they are attached form a 4-14 membered heterocycloalkyl group which is optionally substituted by 1, 2 or 3-W′-X′—Y′-Z′;

or R⁹ is NR^{9a}R^{9b};

R^{9a} and R^{9b} are each, independently, H, C₁₋₆ alkyl, aryl, cycloalkyl, heteroaryl, heterocycloalkyl, each optionally substituted by 1, 2 or 3-W′-X′—Y′-Z′;

or R^{9a} and R^{9b} together with the N atom to which they are attached form a 3-14 membered heterocycloalkyl group which is optionally substituted by 1, 2 or 3-W′-X′—Y′-Z′;

each R¹⁰ is independently OC(O)R^{a′}, OC(O)OR^{b′}, OC(O)NR^{c′}R^{d′}, C(O)OR^{b′}, C(O)NR^{c′}R^{d′}, NR^{c′}R^{d′}, NR^{c′}C(O)R^{a′}, NR^{c′}C(O)OR^{b′}, NR^{c′}S(O)₂R^{b′}, S(O)R^{a′}, S(O)NR^{c′}R^{d′}, S(O)₂R^{a′}, S(O)₂NR^{c′}R^{d′}, OR^{b′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹¹;

or two R¹⁰ together with the same carbon atom to which they are attached form a 3-14 membered cycloalkyl or heterocycloalkyl group which is optionally substituted by 1, 2 or 3 R¹¹;

or two R¹⁰ together with the same carbon atom to which they are attached form a carbonyl group;

or two adjacent R¹⁰ together with the two atoms to which they are attached form a 3-14 membered fused cycloalkyl group or 3-14 membered fused heterocycloalkyl group which is optionally substituted by R¹¹;

or R¹⁰ and -L-Cy together with the same carbon atom to which they are attached form a 3-14 membered cycloalkyl or heterocycloalkyl group which is optionally substituted by 1, 2 or 3 R¹¹;

or adjacent R¹⁰ and -L-Cy together with the two atoms to which they are attached form a 3-14 membered fused cycloalkyl group or 3-14 membered fused heterocycloalkyl group which is optionally substituted by R¹¹;

or R¹⁰ and -L-R^{L} together with the same carbon atom to which they are attached form a 3-14 membered cycloalkyl or heterocycloalkyl group which is optionally substituted by 1, 2 or 3 R¹¹;

or adjacent R¹⁰ and -L-R^{L} together with the two atoms to which they are attached form a 3-14 membered fused cycloalkyl group or 3-14 membered fused heterocycloalkyl group which is optionally substituted by R¹¹;

or adjacent R⁴ and R¹⁰ together with the two atoms to which they are attached form a 3-14 membered fused cycloalkyl group or 3-14 membered fused heterocycloalkyl group which is optionally substituted by R¹¹;

or adjacent R⁷ and R¹⁰ together with the two atoms to which they are attached form a 3-14 membered fused cycloalkyl group or 3-14 membered fused heterocycloalkyl group which is optionally substituted by R¹¹;

or adjacent R⁴ and -L-Cy together with the two atoms to which they are attached form a 3-14 membered fused cycloalkyl group or 3-14 membered fused heterocycloalkyl group which is optionally substituted by R¹¹;

or adjacent R⁷ and -L-Cy together with the two atoms to which they are attached form a 3-14 membered fused cycloalkyl group or 3-14 membered fused heterocycloalkyl group which is optionally substituted by R¹¹;

or adjacent R⁴ and -L-R^{L} together with the two atoms to which they are attached form a 3-14 membered fused cycloalkyl group or 3-14 membered fused heterocycloalkyl group which is optionally substituted by R¹¹;

or adjacent R⁷ and -L-R^{L} together with the two atoms to which they are attached form a 3-14 membered fused cycloalkyl group or 3-14 membered fused heterocycloalkyl group which is optionally substituted by R¹¹;

each R¹¹ is independently halo, C₁₋₆ alkyl, C₁₋₆ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};

W, W′, W″ and W^{a} are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

X, X, X″ and X^{a} are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{c}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;

Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};

wherein two -W-X—Y-Z attached to the same atom optionally form a 3-14 membered cycloalkylk or 3-14 membered heterocycloalkyl group optionally substituted by 1, 2 or 3-W″-X″—Y″-Z″;

wherein two -W′-X′—Y′-Z′ attached to the same atom optionally form a 3-14 membered cycloalkyl or 3-14 membered heterocycloalkyl group optionally substituted by 1, 2 or 3-W″-X″—Y″-Z″;

wherein two -W^{a}-X^{a}-W′-X′—Y′-Z′ attached to the same atom optionally form a 3-14 membered cycloalkyl or 3-14 membered heterocycloalkyl group optionally substituted by 1, 2 or 3-W″-X″—Y″-Z″;

wherein -W-X—Y-Z is other than H;

wherein -W′-X′—Y′-Z′ is other than H;

wherein -W^{a}-X^{a}-W′-X″—Y-Z, is other than H;

wherein -W″-X″—Y″-Z″ is other than H;

R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} and R^{f} are independently selected from H, C₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{e} and R^{f} together with the N atom to which they are attached form a 4-, 5-, 6- or 7-membered heterocycloalkyl group;

q is 1, 2 or 3;

q1 is 0, 1 or 2;

q2 is 0, 1 or 2;

s is 0, 1, 2, 3, 4, 5, 6, 7, 8, 9, 10 or 11;

t1 is is 0, 1 or 2; and

t2 is 0, 1 or 2.

In some embodiments, when the compound of the invention has formula Ia and the ring-forming atom J is N, then ring B is other than a ring having the structure: 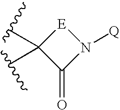
wherein:

Q is —(CR¹⁰¹R¹⁰²)ₘ—R²⁰⁰;

R²⁰⁰ is cycloalkyl, heterocycloalkyl or heteroaryl, each optionally substituted with 1, 2, 3, 4 or 5-W′-X′—Y′-Z′;

E is —(CR^{103a}R^{103b})ₙ₁—, —(CR^{103a}R^{103b})ₙ₂CO—, —(CR^{103a}R^{103b})ₙ₂OCO—, —(CR^{103a}R^{103b})ₙ₂SO—, —(CR^{103a}R^{103b})ₙ₂SO₂—, —(CR^{103a}R^{103b})ₙ₂NR^{103c}—, —(CR^{103a}R^{103b})ₙ₂CONR^{103c}—, —(CR^{103a}R^{103b})ₙ₂NR^{103c}CO—, or a group of formula:

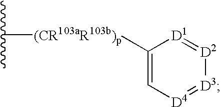

D¹, D², D³ and D⁴ are independently selected from N and CR¹⁰⁴;

R¹⁰¹ and R¹⁰² are independently selected from H and C₁₋₈ alkyl;

R^{103a} and R^{103b} are independently selected from H, halo, C₁₋₄ alkyl, C₁₋₄ haloalkyl, C₂₋₄ alkenyl, and C₂₋₄ alkynyl;

R^{103c} is H, C₁₋₄ alkyl, C₁₋₄ haloalkyl, C₂₋₄ alkenyl, C₂₋₄ alkynyl, or CO—(C₁₋₄ alkyl);

each R¹⁰⁴ is independently H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

m is 0, 1, 2 or 3;

n1 is 1, 2, 3 or 4;

n2is 0, 1, 2, 3 or 4; and

p is 0, 1 or 2.

In some embodiments, Cy is aryl or heteroaryl, each optionally substituted with 1, 2, 3, 4 or 5-W-X—Y-Z.

In some embodiments, Cy is aryl or heteroaryl, each optionally substituted with 1, 2, 3, 4 or 5-W-X—Y-Z wherein W is O or absent, X is absent, and Y is absent.

In some embodiments, Cy is phenyl, naphthyl, pyridyl, pyrimidinyl, triazinyl, furanyl, thiazolyl, pyrazinyl, purinyl, quinazolinyl, quinolinyl, isoquinolinyl, pyrrolo[2,3-d]pyrimidinyl, or 1,3-benzothiazolyl, each optionally substituted with 1, 2, 3, 4 or 5-W-X—Y-Z.

In some embodiments, Cy is phenyl, naphthyl, pyridyl, pyrimidinyl, triazinyl, furanyl thiazolyl, pyrazinyl, purinyl, quinazolinyl, quinolinyl, isoquinolinyl, pyrrolo[2,3-d]pyrimidinyl, or 1,3-benzothiazolyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, CN, NO₂, OR^{a}, SR^{a}, C(O)NR^{c}R^{d}, NR^{c}C(O)R^{d} and COOR^{a}.

In some embodiments, Cy is phenyl, pyridyl, pyrimidinyl, pyrazinyl or 1,3-benzothiazolyl, each optionally substituted by 1, 2, 3, 4 or 5-W-X—Y-Z.

In some embodiments, Cy is phenyl, pyridyl, pyrimidinyl, pyrazinyl or 1,3-benzothiazolyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, CN, NO₂, OR^{a}, SR^{a}, C(O)NR^{c}R^{d}, NR^{c}C(O)R^{d} and COOR^{a}.

In some embodiments, Cy is phenyl, pyridyl, pyrimidinyl, pyrazinyl or 1,3-benzothiazolyl, each optionally substituted by 1, 2, 3 or 4 substituents independently selected from halo, CN, C₁₋₆ haloalkyl, C₁₋₆ alkyl and aryl, wherein each of the C₁₋₆ alkyl and aryl is optionally substituted by 1, 2 or 3 substituents independently selected from halo, C₁₋₆ alkyl, C₁₋₆ haloalkyl and CN.

In some embodiments, Cy is cycloalkyl or heterocycloalkyl, each optionally substituted by 1, 2, 3, 4 or 5-W-X—Y-Z.

In some embodiments, Cy is cycloalkyl or heterocycloalkyl, each optionally substituted by 1, 2, 3, 4 or 5-W-X—Y-Z wherein W is O or absent, X is absent, and Y is absent.

In some embodiments, cyclopropyl, cyclobutyl, cyclopentyl, cyclohexyl, cyclocheptyl, adamantyl, aziridinyl, azetidinyl, pyrrolidine, piperidinyl, 2-oxo-hexahydropyrimidinyl, piperizinyl or morpholinyl, each optionally substituted by 1, 2, 3, 4 or 5-W-X—Y-Z.

In some embodiments, Cy is cyclopropyl, cyclobutyl, cyclopentyl, cyclohexyl, cyclocheptyl, adamantyl, aziridinyl, azetidinyl, pyrrolidine, piperidinyl, 2-oxo-hexahydropyrimidinyl, piperizinyl or morpholinyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, CN, NO₂, OR^{a}, SR^{a}, C(O)NR^{c}R^{d}, NR^{c}C(O)R^{d} and COOR^{a}.

In some embodiments, Cy is cyclohexyl or piperidinyl each optionally substituted by 1, 2, 3, 4 or 5-W-X—Y-Z.

In some embodiments, L is absent.

In some embodiments, L is (CR¹R²)_{q1}S(CR¹R²)_{q2}, (CR¹R²)_{q1}SO₂(CR¹R²)_{q2}, (CR¹R²)_{q1}SO(CR¹R²)_{q2} or (CR¹R²)_{q1}SO₂NR³(CR¹R²)_{q2}.

In some further embodiments, L is (CR¹R²)_{q1}S(CR¹R²)_{q2} or (CR¹R²)_{q1}SO₂(CR¹R²)_{q2}.

In some embodiments, L is S, SO, SO₂ or SO₂NH. In some further embodiments, L is S or SO₂. In yet further embodiments, L is SO₂.

In some embodiments, L is (CR¹R²)_{q1}COO(CR¹R²)_{q2}, (CR¹R²)_{q1}CO(CR¹R²)_{q2}, (CR¹R²)_{q1}NR^{3a}CONR³(CR¹R²)_{q2}, or (CR¹R²)_{q1}CONR³(CR¹R²)_{q2}. In some further embodiments, L is COO, CO, COO—C₁₋₃ alkylene, NR^{3a}CONR³ or CONR³. In yet further embodiments, L is COO, CO, COO—C₁₋₃ alkylene, NHCONH, N(C₁₋₄ alkyl)CONH, N(C₁₋₄ alkyl)CON(C₁₋₄ alkyl), or CONH, CON(C₁₋₄alkyl).

In some embodiments, L is (CR¹R²)_{q1}O(CR¹R²)_{q2}. In some further embodiments, L is O.

In some embodiments, L is (CR¹R²)_{q}. In some further embodiments, L is C₁₋₃ alkylene.

In some embodiments, t1 is 0.

In some embodiments, t1 is 1 or 2. In some embodiments, t1 is 1.

In some embodiments, t2 is 0.

In some embodiments, t2 is 1 or 2. In some further embodiments, t2 is 1. In some other embodiments, t2 is 2.

In some embodiments, M¹ is CH or CH₂.

In some embodiments, M¹ is C(O), O, SO₂, OC(O), NH, NHC(O), or NHSO₂. In some further embodiments, M¹ is C(O), O, SO₂, OC(O), or NH.

In some embodiments, M² and M³ are independently selected from absent, C(O), OC(O), O, NH, and SO₂.

In some embodiments, one of M² and M³ is absent, and the other is selected from C(O), OC(O), O, NH, and SO₂.

In some embodiments, each R¹⁰ is independently OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, OR^{b′}, SR^{b′}, C₁₋₁₀ alkyl, C₁₋₁₀ haloalk, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

In some embodiments, each R¹⁰ is independently C(O)OR^{b′}, C₁₋₁₀ alkyl or C₁₋₁₀ haloalkyl.

In some embodiments, s is 0, 1, 2, or 3. In some embodiments, s is 0, 1 or 2. In some further embodiments, s is 0 or 1. In yet further embodiments, s is 0.

In some embodiments, the compounds of the present invention have formula Ia.

In some embodiments of compounds of formula Ia, the ring-forming atom J is N.

In some embodiments of compounds of formula Ia, the ring-forming atom J is C.

In some embodiments of compounds of formula Ia:

ring B is selected from:

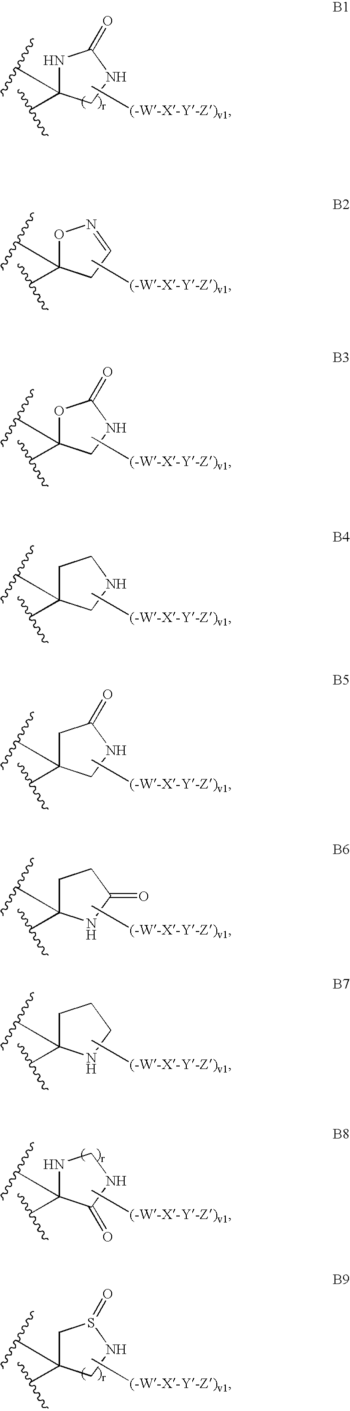

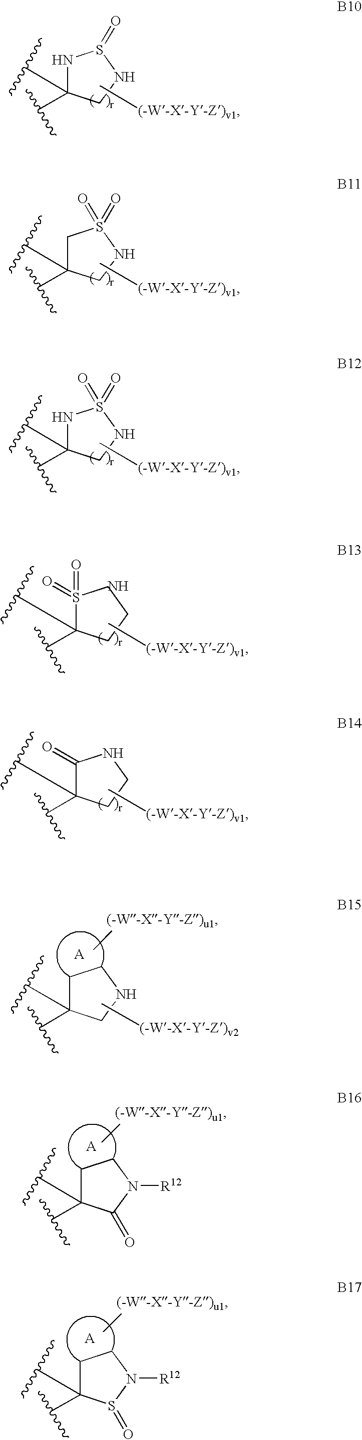

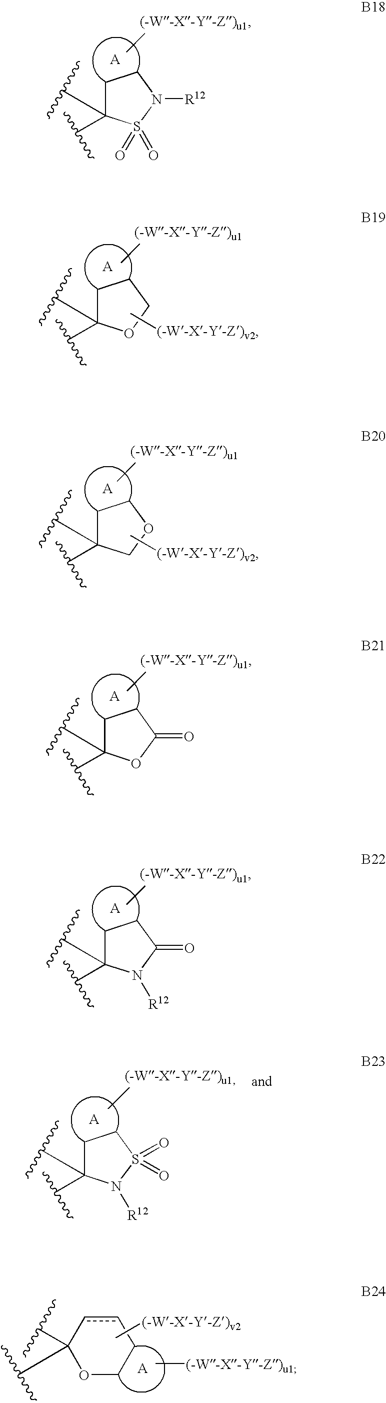

[symbol] is a single or double bond;

r is 0, 1 or 2;

v1 is 0, 1, 2, or 3;

v2 is 0 or 1;

u1 is 0, 1, 2, or 3;

each R¹² is H or -W′-X′—Y′-Z′; and

ring A is a 5- or 6-membered aryl or heteroaryl.

In some embodiments of compounds of formula Ia:

ring B is selected from:

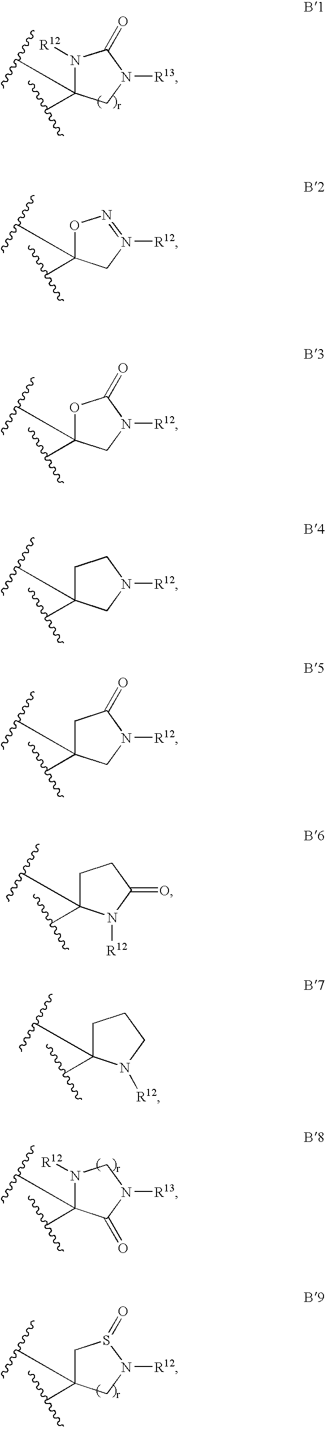

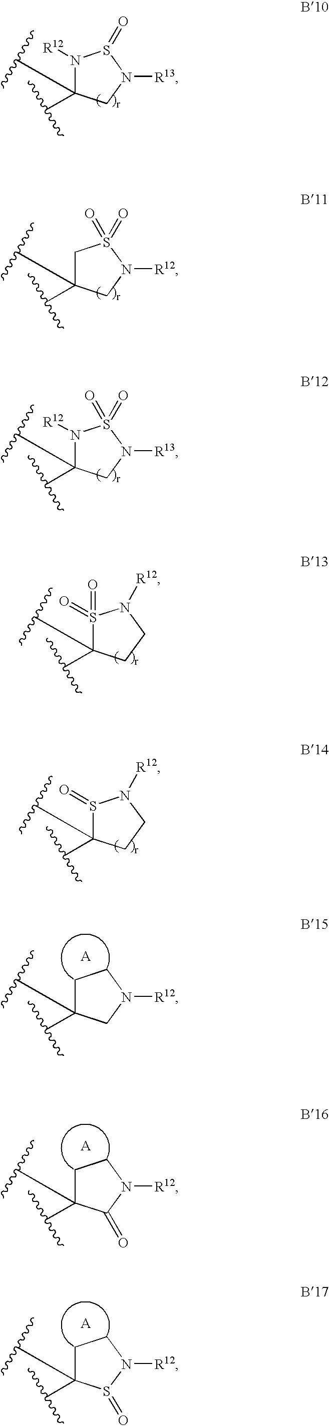

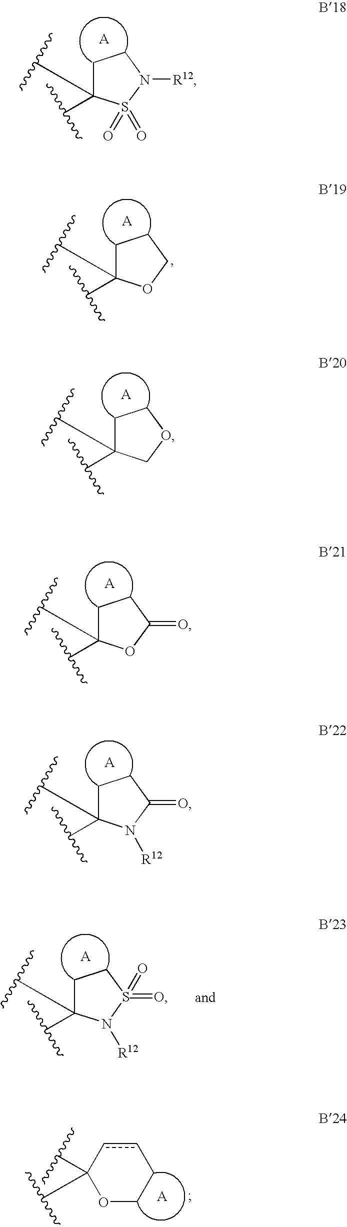

r is 0, 1 or 2;

each R¹² is H or -W′-X′—Y′-Z′;

each R¹³ is H or -W′-X′—Y′-Z′; and

ring A is a 5- or 6-membered aryl or heteroaryl.

In some embodiments, each of R¹² and R¹³ is independently H, C(O)R^{b}, COOR^{a}, C(O)NR^{c}R^{d}, S(O)₂R^{b}, S(O)₂NR^{c}R^{d}, or C₃₋₁₄ cycloalkyl, wherein the C₃₋₁₄ cycloalkyl is optionally substituted by 1 or 2 substituents independently selected from C₁₋₆ alkyl, C₁₋₆ halolkyl, OH, C₁₋₆ alkoxy, heteroaryloxy, C₁₋₆ haloalkoxy, aryl and heteroaryl, and wherein each of aryl and heteroaryl is optionally substituted by 1 or 2 substituents independently selected from halo, C₁₋₆ alkyl, C₁₋₆ halolkyl and C₁₋₆ haloalkoxy.

In some embodiments, ring B has the structure of B′1, B′2, B′4, B5′, B′16, B′19, B′21 or B′24.

In some embodiments, ring B has the structure of B′5, B′13 or B′14; and R¹² is C₃₋₁₄ cycloalkyl optionally substituted by 1 or 2 substituents independently selected from aryl, heteroaryl, C₁₋₆ alkyl, C₁₋₆ halolkyl, C₁₋₆ hydroxyalkyl, OH, C₁₋₆ alkoxy, C₁₋₆ haloalkoxy, C₂₋₁₂ alkoxyalkoxy, aryloxy and heteroaryloxy.

In some embodiments, ring B has the structure of B′5, B′13 or B′14; and R¹² is C₃₋₁₄ cycloalkyl optionally substituted by 1 or 2 substituents independently selected from C₁₋₆ alkyl, C₁₋₆ halolkyl, C₁₋₆ hydroxyalkyl, OH, C₁₋₆ alkoxy, C₁₋₆ haloalkoxy, C₂₋₁₂ alkoxyalkoxy, aryloxy and heteroaryloxy.

In some embodiments of compounds of formula Ia, R^{L} is Cy. In some other embodiments of compounds of formula Ia, R^{L} is C₁₋₆ alkyl optionally substituted by 1, 2, 3, 4 or 5-W-X—Y-Z;

In some embodiments of compounds of formula Ia, L is absent, CO, CONH, COO, or SO₂.

In some embodiments of compounds of formula Ia, the compound has the formula: 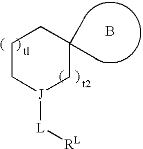
wherein:

t1 is 0 or 1; and

t2 is 1 or 2.

In some embodiments of compounds of formula Ia:

t1 is 1;

t2 is 1;

L is absent;

R^{L} is Cy.

In some embodiments of compounds of formula Ia, ring B is selected from: 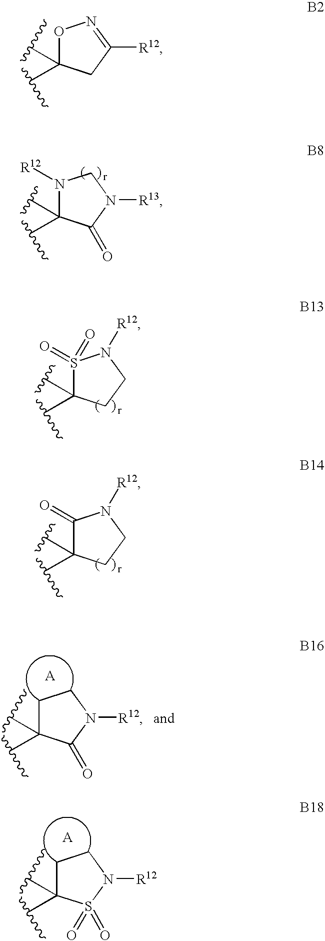
wherein:

r is 0, 1 or 2;

each R¹² is H or -W′-X′—Y′-Z′;

each R¹³ is H or -W′-X′—Y′-Z′; and

ring A is a 5- or 6-membered aryl or heteroaryl.

In some embodiments, the compounds of the present invention have formula Ib, Id or If. In some further embodiments, the compounds of the present invention have formula Ib.

In some embodiments, R⁴ is H, C(O)OR^{b′} or C₁₋₁₀ alkyl.

In some embodiments, R⁴ is H or C₁₋₁₀ alkyl.

In some embodiments, R⁵ is cycloalkyl or heterocycloalkyl, each optionally substituted by 1, 2 or 3-W′-X′—Y′-Z′.

In some embodiments, R⁵ is cycloalkyl optionally substituted by 1, 2 or 3 substituents independently selected from C₁₋₆ alkyl, C₁₋₆ halolkyl, C₁₋₆ hydroxyalkyl, OH, C₁₋₆ alkoxy, C₁₋₆ haloalkoxy, C₂₋₁₂ alkoxyalkoxy, aryloxy and heteroaryloxy.

In some embodiments, R⁶ is H or C₁₋₁₀ alkyl.

In some embodiments of compounds of formula Ia:

ring B is selected from:

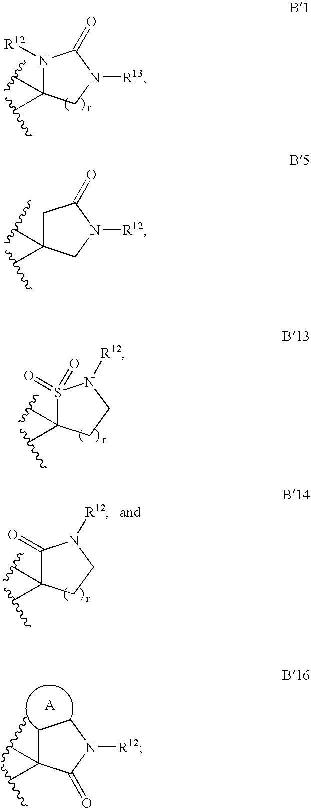

each R¹² is H or -W′-X′—Y′-Z′;

R¹³ is H or -W′-X′—Y′-Z′;

r is 0, 1, or 2;

the ring-forming atom J is C;

L is absent, O or S;

t1 is 0; and

t2 is 1 or 2.

In some embodiments of compounds of formula Ia:

ring B is selected from:

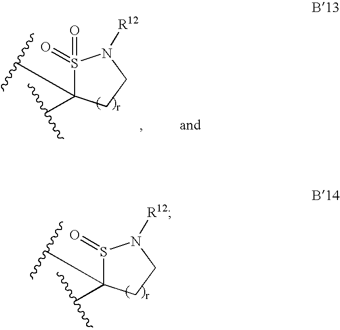

the ring-forming atom J is C;

R¹² is H or -W′-X′—Y′-Z′;

L is absent, O or S;

t1 is 0; and

t2 is 2.

In some embodiments of compounds of formula Ia:

ring B is selected from:

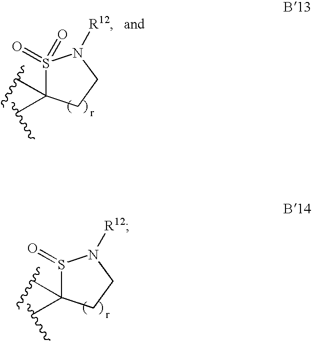

the ring-forming atom J is C;

R¹² is H or -W′-X′—Y′-Z′;

L is absent, O, S or SO₂;

t1 is 1; and

t2 is 1.

In some embodiments of compounds of formula Ia:

ring B has the structure:

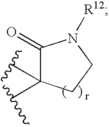

the ring-forming atom J is C;

R¹² is H or -W′-X′—Y′-Z′;

L is absent, CH₂, O, S or SO₂;

t1 is 0; and

t2 is 2.

In some embodiments of compounds of formula Ia:

ring B has the structure:

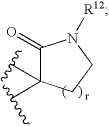

the ring-forming atom J is C;

R¹² is H or -W′-X′—Y′-Z′;

L is absent, CH₂, O, S or SO₂;

t1 is 1; and

t2 is 1.

In some embodiments, the compounds of of invention have formula Ib, Id or If.

In some embodiments of compounds of formula Ib, Id or If, L is absent, O, C₁₋₃ alkylene, CO, NHCONH, N(C₁₋₄ alkyl)CONH, N(C₁₋₄ alkyl)CON(C₁₋₄ alkyl), CONH, CON(C₁₋₄ alkyl), COO, S, or SO₂.

In some embodiments of compounds of formula Ib, Id or If, L is absent, O, C₁₋₃ alkylene, CO, CONH, CON(C₁₋₄alkyl), COO, S, or SO₂.

In some embodiments, the compounds of the invention have formula Ib.

In some embodiments of compounds having formula Ib, the compounds have formula:

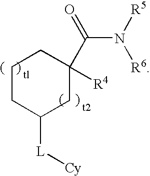

In some embodiments of compounds having formula Ib:

t1 is 0 or 1;

t2 is 1 or 2; and

L is absent, (CR¹R²)_{q}, (CR¹R²)_{q1}O(CR¹R²)_{q2}, (CR¹R²)_{q1}S(CR¹R²)_{q2}, or (CR¹R²)_{q1}SO₂(CR¹R²)_{q2}, (CR¹R²)_{q1}SO(CR¹R²)_{q2},

In some embodiments of compounds having formula Ib, R⁴ and R⁵ together with the intervening —C—C(O)—N(R⁶)— moiety to which they are attached form a 4-14 membered heterocycloalkyl group which is optionally substituted by 1, 2 or 3-W′-X′—Y′-Z′.

In some embodiments of compounds of formulaIb, R⁴ is H; L is absent, CH₂, O, S or SO₂; R⁵ is cycloalkyl or heterocycloalkyl, each optionally substituted by 1, 2 or 3-W′-X′—Y′-Z′; and R⁶ is H or C₁₋₆ alkyl.

In some embodiments of compounds of formula Ib, R⁴ is H; L is absent, CH₂, O, S or SO₂; R⁵ is cycloalkyl optionally substituted by 1, 2 or 3-W′-X′—Y′-Z′; and R⁶ is H.

In some embodiments of compounds of formula Ib, R⁴ is H; L is absent, CH₂, O, S or SO₂; R⁵ is cycloalkyl optionally substituted by 1, 2 or 3 substituents independently selected from OH and CN; and R⁶ is H.

In some embodiments, the compounds of present invention have formula formula Ic or Ie.

In some embodiments, R⁷ is H, C(O)OR^{b} or C₁₋₁₀ alkyl.

In some embodiments, R⁹ is H, C₁₋₆ alkyl, arylalkyl, heteroarylalkyl, cycloalkyl, cycloalkylalkyl, heterocycloalkyl or heterocycloalkylalkyl, wherein each of the C₁₋₆ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′.

In some embodiments, R⁹ is NR^{9a}R^{9b}; R^{9a} is H or C₁₋₆ alkyl; and R^{9b} is cycloalkyl or heterocycloalkyl, each optionally substituted by 1, 2 or 3-W′-X′—Y′-Z′.

In some embodiments, R⁹ is NR^{9a}R^{9b}; R^{9a} is H or C₁₋₆ alkyl; and R^{9b} is cycloalkyl or heterocycloalkyl, each optionally substituted by 1, 2 or 3 substituents independently selected from C₁₋₆ alkyl, C₁₋₆ halolkyl, C₁₋₆ hydroxyalkyl, OH, C₁₋₆ alkoxy, C₁₋₆ haloalkoxy and C₂₋₈ alkoxyalkoxy.

In some embodiments, T is O or CH₂.

In some embodiments, T is NR⁸; and R⁸ is H or C₁₋₁₀ alkyl.

In some embodiments, T is NR⁸; and R⁸ and R⁹ together with the two adjacent atoms to which they are attached form a 3-14 membered heterocycloalkyl group which is optionally substituted by 1, 2 or 3-W′-X′—Y′-Z′.

In some embodiments, the compounds of the invention have formula Ic.

In some embodiments of compounds of formula Ic, the compounds have the formula:

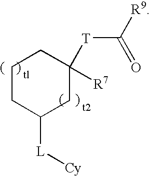

In some embodiments of compounds of formula Ic:

t1 is 0 or 1;

t2 is 1 or 2;

L is absent, (CR¹R²)_{q}, (CR¹R²)_{q1}O(CR¹R²)_{q2}, (CR¹R²)_{q1}S(CR¹R²)_{q2}, or (CR¹R²)_{q1}SO₂(CR¹R²)_{q2}, or (CR¹R²)_{q1}SO(CR¹R²)_{q2}.

In some embodiments of compounds of formula Ic, R⁸ and R⁹ together with the two adjacent atoms to which they are attached form a 3-14 membered heterocycloalkyl group which is optionally substituted by 1, 2 or 3-W′-X′—Y′-Z′.

In some embodiments of compounds of formula Ic, R⁷ is H; L is absent, CH₂, O, S or SO₂; and T is NR⁸.

In some embodiments of compounds of formula Ic, R⁷ is H; L is absent, CH₂, O, S or SO₂; and T is NH.

In some embodiments of compounds of formula Ic, R⁷ is H; L is absent, CH₂, O, S or SO₂; T is NH; and R⁹ is NR^{9a}R^{9b}.

In some embodiments of compounds of formula Ic, R⁷ is H; L is absent, CH₂, O, S or SO₂; T is NH; R⁹ is NR^{9a}R^{9b}; R^{9a} is H or C₁₋₆ alkyl; and R^{9b} is cycloalkyl or heterocycloalkyl, each optionally substituted by 1, 2 or 3-W′-X′—Y′-Z′.

In some embodiments of compounds of formula Ic, R⁷ is H; L is absent, CH₂, O, S or SO₂; T is NH; and R⁸ and R⁹ together with the two adjacent atoms to which they are attached form a 3-14 membered heterocycloalkyl group which is optionally substituted by 1, 2 or 3-W′-X′—Y′-Z′.

In some embodiments, the compounds of the invention have formula Id.

In some embodiments, the compounds of the invention have formula Ie.

In some embodiments, the compounds of the invention have formula If.

In some embodiments of compounds having formula If, the compounds have the formula:

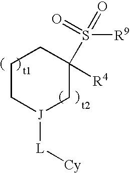

wherein:

t1 is 0 or 1;

t2 is 1 or 2; and

L is absent, (CR¹R²)_{q}, (CR¹R²)_{q1}O(CR¹R²)_{q2}, (CR¹R²)_{q1}S(CR¹R²)_{q2}, or (CR¹R²)_{q1}SO₂(CR¹R²)_{q2}, or (CR¹R²)_{q1}SO(CR¹R²)_{q2}.

In some embodiments, the compounds of the invention have formula Ig.

In some embodiments, each -W-X—Y-Z is independently selected from halo, nitro, cyano, OH, C₁₋₄ alkyl, C₁₋₄ haloalkyl, C₁₋₄ hydroxyalkyl, C₁₋₄ cyanoalkyl, C₂₋₈ alkoxyalkoxy, C₁₋₄ haloalkoxy, amino, C₁₋₄ alkoxy, cycloalkylcarbonylamino, alkoxycarbonylamino, alkylsulfonylamino, cycloalkylalkylcarbonylamino, acyl(alkyl)amino, alkylamino, dialkylamino, dialkylaminosulfonyl, dialkylaminocarbonyl, dialkylaminocarbonylalkyloxy, alkylcarbonyl(alkyl)amino, cycloalkylcarbonyl(alkyl)amino, alkoxycarbonyl(alkyl)amino, alkoxycarbonyl, alkylsulfonyl, arylsulfonyl, aryl, cycloalkyl, heteroaryl, heterocycloalkyl, aryloxy, cycloalkyloxy, heteroaryloxy, heterocycloalkyloxy, arylalkyloxy, and acylamino;

wherein each of said aryl, cycloalkyl, heteroaryl, heterocycloalkyl, arylalkyloxy and heterocycloalkyloxy is optionally substituted by 1 or more substituents independently selected from halo, C₁₋₄ alkyl, OH, C₁₋₄ alkoxy, C₁₋₄ hydroxyalkyl, C₁₋₄ cyanoalkyl, C₂₋₈ alkoxyalkoxy, cycloalkylaminocarbonyl, alkoxycarbonyl, cyano, acyl, acylamino, alkylsulfonyl, amino, alkylamino, dialkylamino, and aminocarbonyl.

In some embodiments, each -W-X—Y-Z is independently selected from halo, CN, NO₂, C₁₋₄ alkoxy, heteroaryloxy, C₂₋₆ alkynyl, C₁₋₄ haloalkoxy, NR^{c}C(O)R^{d}, NR^{c}C(O)OR^{a}, C(O)NR^{c}R^{d}, NR^{c}R^{d}, NR^{c}S(O)₂R^{b}, C₁₋₄ haloalkyl, C₁₋₆ alkyl, heterocycloalkyl, aryl and heteroaryl, wherein each of said C₁₋₆ alkyl, aryl and heteroaryl is optionally substituted by 1, 2 or 3 substituents independently selected from halo, C₁₋₆ alkyl, C₁₋₄ haloalkyl, CN, NO₂, OR^{a}, SR^{a}, C(O)NR^{c}R^{d}, NR^{c}C(O)R^{d} and COOR^{a}.

In some embodiments, each -W′-X′—Y′-Z′ is independently selected from halo, OH, cyano, nitro, C₁₋₄ alkyl, C₁₋₄ alkoxy, C₁₋₄ haloalkyl, C₁₋₄ hydroxyalkyl, C₁₋₄ cyanoalkyl, C₂₋₈ alkoxyalkoxy, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl;

wherein each of said aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl and heterocycloalkyloxy is optionally substituted by 1 or 2 substituents independently selected from halo, OH, cyano, nitro, C₁₋₄ alkyl, C₁₋₄ alkoxy, C₁₋₄ haloalkyl, C₁₋₄ hydroxyalkyl, C₁₋₄ cyanoalkyl, C₂₋₈ alkoxyalkoxy, amino, alkylamino, dialkylamino, and alkoxycarbonyl.

In some embodiments, each 13 W′-X′—Y′-Z′ is independently selected from halo, OH, cyano, nitro, C₁₋₄ alkyl, C₁₋₄ alkoxy, C₁₋₄ haloalkyl, C₁₋₄ haloalkoxy, C₁₋₄ hydroxyalkyl, C₁₋₄ cyanoalkyl, C₂₋₈ alkoxyalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl.

In some embodiments, each -W^{a}-X^{a}-W′-X′—Y′-Z′ is independently selected from halo, OH, cyano, nitro, C₁₋₄ alkyl, C₁₋₄ alkoxy, C₁₋₄ haloalkyl, C₁₋₄ hydroxyalkyl, C₁₋₄ cyanoalkyl, C₂₋₈ alkoxyalkoxy, C₁₋₄ haloalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl;

wherein each of said aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl and heterocycloalkyloxy is optionally substituted by 1 or 2 substituents independently selected from halo, OH, cyano, nitro, C₁₋₄ alkyl, C₁₋₄ alkoxy, C₁₋₄ haloalkyl, C₁₋₄ hydroxyalkyl, C₁₋₄ cyanoalkyl, C₂₋₈ alkoxyalkoxy, amino, alkylamino, dialkylamino, and alkoxycarbonyl.

In some embodiments, each -W″-X″—Y″-Z″ is independenly selected from halo, OH, cyano, nitro, C₁₋₄ alkyl, C₁₋₄ alkoxy, C₁₋₄ haloalkyl, C₁₋₄ haloalkoxy, C₁₋₄ hydroxyalkyl, C₁₋₄ cyanoalkyl, C₂₋₈ alkoxyalkoxy, amino, alkylamino, dialkylamino, hydroxylalkyl, aryl, arylalkyl, aryloxy, heteroaryl, heteroarylalkyl, heteroaryloxy, cycloalkyl, cycloalkylalkyl, cycloalkyloxy, heterocycloalkylalkyl, heterocycloalkylalkyl, heterocycloalkyloxy, aminocarbonyl, alkylaminocarbonyl, dialkylaminocarbonyl, alkylcarbonyloxy, alkylsulfonyl, and arylsulfonyl.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defming the variable. For example, where a structure is described having two R groups that are simultaneously present on the same compound; the two R groups can represent different moieties selected from the Markush group defined for R. In another example, when an optionally multiple substituent is designated in the form: 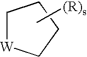
then it is understood that substituent R can occur s number of times on the ring, and R can be a different moiety at each occurrence. Further, in the above example, should the variable W be defined to include hydrogens, such as when W is said to be CH₂, NH, etc., any floating substituent such as R in the above example, can replace a hydrogen of the W variable as well as a hydrogen in any other non-variable component of the ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylene” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” or “alkenylene” refers to a linking alkenyl group between two moieties in a molecule. Also emcompassed in the definition of alkenylenyl or alkenylene is a moiety of formula: 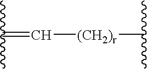
wherein r is an integer such as 0, 1, 2 or 3. Thus, a C₁ akenyleny is a moiety having the formula of 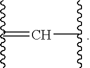
The alkenylenyl groups, like all other groups, can further be substituted as described herein.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, CH₂CF₃, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “heteroaryl” groups refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles where one or more of the ring-forming atoms is replaced by a heteroatom such as an O, N, or S atom. Hetercycloalkyl groups can be mono or polycyclic (e.g., both fused and spiro systems). Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to an —O-haloalkyl group. An example haloalkoxy group is OCF₃.

As used herein, “alkoxyalkyl” refers to an alkyl group substituted by an alkoxy group. One example of alkoxyalkyl is —CH₂—OCH₃.

As used herein, “cyanoalkyl” refers to an alkyl group substituted by a cyano group (CN). One example of cyanoalkyl is —CH₂—CN.

As used herein, “alkoxyalkoxy” refers to an alkoxy group substituted by an alkoxy group. One example of alkoxyalkoxy is —OCH₂CH₂—OCH₃.

As used herein, “arylalkyl” refers to alkyl substituted by aryl and “cycloalkylalkyl” refers to alkyl substituted by cycloalkyl. An example arylalkyl group is benzyl. As used herein, “arylalkenyl” refers to alkenyl substituted by aryl and “arylalkynyl” refers to alkynyl substituted by aryl.

As used herein, “heteroarylalkyl” refers to an alkyl group substituted by a heteroaryl group, and “heterocycloalkylalkyl” refers to alkyl substituted by heterocycloalkyl. As used herein, “heteroarylalkenyl” refers to alkenyl substituted by heteroaryl and “heteroarylalkynyl” refers to alkynyl substituted by heteroaryl.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “dialkylaminocarbonyl” refers to a carbonyl group substituted by a dialkylamino group.

As used herein, “dialkylaminocarbonylalkyloxy” refers to an alkyloxy (alkoxy) group substituted by a carbonyl group which in turn is substituted by a dialkylamino group.

As used herein, “cycloalkylcarbonyl(alkyl)amino” refers to an alkylamino group substituted by a carbonyl group (on the N atom of the alkylamino group) which in turn is substituted by a cycloalkyl group. The term “cycloalkylcarbonylamino” refers to an amino group substituted by a carbonyl group (on the N atom of the amino group) which in turn is substituted by a cycloalkyl group. The term “cycloalkylalkylcarbonylamino” refers to an amino group substituted by a carbonyl group (on the N atom of the amino group) which in turn is substituted by a cycloalkylalkyl group.

As used herein, “alkoxycarbonyl(alkyl)amino” refers to an alkylamino group substituted by an alkoxycarbonyl group on the N atom of the alkylamino group. The term “alkoxycarbonylamino” refers to an amino group substituted by an alkoxycarbonyl group on the N atom of the amino group.

As used herein “alkoxycarbonyl” refers to a carbonyl group [—C(O)—] substituted by an alkoxy group.

As used herein, “alkylsulfonyl” refers to a sulfonyl group [—S(O)₂—] substituted by an alkyl group. The term “alkylsulfonylamino” refers to an amino group substituted by an alkylsulfonyl group.

As used herein, “arylsulfonyl” refers to a sulfonyl group [—S(O)₂—] substituted by an aryl group, i.e., —S(O)₂-aryl.

As used herein, “dialkylaminosulfonyl” refers to a sulfonyl group substituted by dialkylamino.

As used herein, “arylalkyloxy” refers to —O-arylalkly. An example of an arylalkyloxy group is benzyloxy.

As used heren, “cycloalkyloxy” refers to —O-cycloalkyl. An example of a cycloalkyloxy group is cyclopenyloxyl.

As used herein, “heterocycloalkyloxy” refers to —O-heterocycloalkyl.

As used herein, “aryloxy” refers to —O-aryl. An example of aryloxy is phenoxy. The term “aryloxyalkyl” refers to an alkyl group substituted by an aryloxy group.

As used herein, “heteroaryloxy” refers to —O-heteroaryl. An example is pyridyloxy. The term “heteroaryloxyalkyl” refers to an alkyl group substituted by a heteroaryloxy group.

As used herein, “acylamino” refers to an amino group substituted by an alkylcarbonyl (acyl) group. The term “acyl(alkyl)amino” refers to an amino group substituted by an alkylcarbonyl (acyl) group and an alkyl group.

As used herein, “alkylcarbonyl” refers to a carbonyl group substituted by an alkyl group.

As used herein, “cycloalkylaminocarbonyl” refers to a carbonyl group substituted by an amino group which in turn is substituted by a cycloalkyl group.

As used herein, “aminocarbonyl” refers to a carbonyl group substituted by an amino group (i.e., CONH₂).

As used herein, “hydroxyalkyl” refers to an alkyl group substituted by a hydroxyl group. An example is —CH₂OH.

As used herein, “alkylthio” refers to —S-alkyl, and “methylthio” refers to —S—CH₃.

As used herein, “alkylcarbonyloxy” refers to an oxy group substituted by a carbonyl group which in turn is substituted by an alkyl group [i.e., —O—C(O)-(alkyl)].

As used herein, the terms “substitute” or “substitution” refer to replacing a hydrogen with a non-hydrogen moiety.

As used used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group (i.e., CH₃) is optionally substituted, then 3 hydrogens on the carbon atom can be replaced with substituent groups.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention are intended to include compounds with stable structures. As used herein, “stable compound” and “stable structure” are meant to indicate a compound that is sufficiently robust to survive isolation to a useful degree of purity from a reaction mixture, and formulation into an efficacious therapeutic agent.

The term, “compound,” as used herein is meant to include all stereoisomers, geometric iosomers, tautomers, and isotopes of the structures depicted.

All compounds, and pharmaceuticaly acceptable salts thereof, are also meant to include solvated or hydrated forms.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C NMR), infrared spectroscopy (IR), spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

As shown in Scheme 1 a series of compounds of general formula 1-2 can be prepared by the reaction of a secondary amine 1-1 (or salts thereof) with an electrophilic species such as an alkyl or acyl halide R^{L}LX¹ (X¹ is, e.g., I, Cl, Br, OTf, OTs, etc.; L is absent or CO; R^{L} is alkyl, cycloalkyl and the like) in the presence of a suitable base such as diisopropylethylamine (DIPEA) in an appropriate solvent (eg. CH₂Cl₂). Alternatively, the secondary amine 1-1 can be converted to a sulfonamide of general formula 1-3 by reaction with an appropriate sulfonyl chloride R^{L}SO₂Cl in the present of a suitable base such as Hunig's base, and to a urea of general formula 1-4 by a two step protocol, in which the amine 1-1 is first treated with p-nitrophenyl chloroformate in the presence of a suitable base, such as Hunig's base, to form an activated species such as carbamate followed by reacting with a suitable amine HR^{L1}R^{L2} to afford a urea of general formula 1-4.

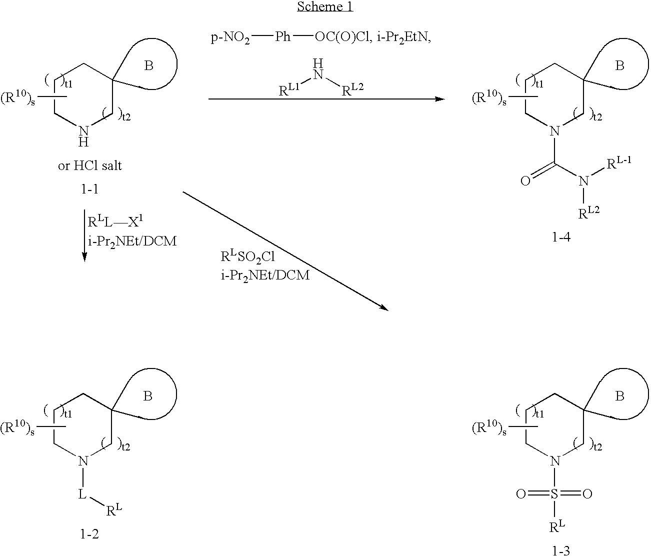

In addition to the standard S_{N}2 reaction such as one shown in Scheme 1 (between an alkyl halide and an amine), a secondary amine 2-1 (or salt thereof) can undergo substitution by reductive amination methods by treatment of amine 2-1 with an aldehyde or ketone RC(O)R′ (wherein R and R′ are H, alkyl, aryl or the like; or R and R′ together with the carbon atom to which they are attached form a 3-14 membered alkyl or heteroalkyl, which is optionally substituted by one or more substitutents such as alky, halo, etc.) and an appropriate reducing reagent such as sodium triacetoxyborohydride or sodium cyanoborohydride in a suitable solvent such as dichloromethane or dichloroethane to afford compound 2-2 as shown below in Scheme 2.

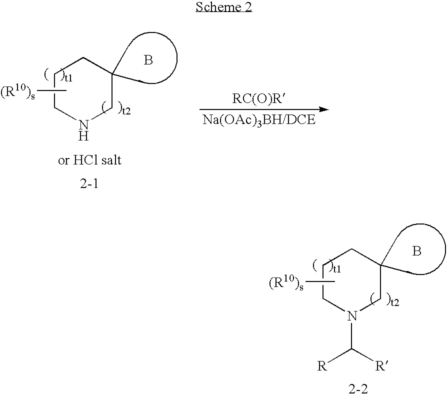

As shown in Scheme 3, a compound of general formula 3-2 can be obtained by reaction of a secondary amine 3-1 (or salt thereof) with an aryl halide or heteroaryl halide Ar—X¹ (wherein Ar is substituted or unsubstituted aryl or hetereoaryl and X¹ is halo such as chloride or bromide) The reaction can be carried out under a suitable condition such as at an elevated temperature, in the presence of a suitable base such as potassium carbonate, potassium phosphate, or sodium tert-butoxide, in the absence or presence of an organometallic catalyst such as palladium (0) or zinc (II) complex, and in a polar aprotic solvent such as DMF or DMSO (See, e.g., Cho, G. Y. et al. J. Org. Chem. 2005, 70, 2346; Nie, Z. et al. J. Med. Chem. 2005, 48, 1596). If the nitrogen-containing-ring A¹ is a lactam (i.e., two R¹⁰ together with the same carbon atom to which they are attached form a carbonyl, i.e., C═O, and the carbonyl is adjacent to the nitrogen atom in ring A¹) then an Ullman-Ukita-Buchwald-lithium reaction can be implemented using CuI as described by Wang, P.-S. et al. Tetrahedron 2005, 61, 2931.

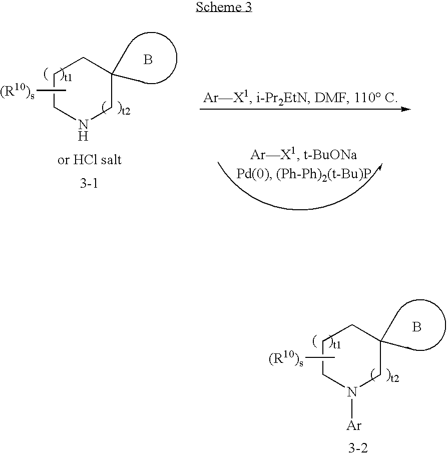

As shown in Scheme 4, a halogenated compound 4-1 (wherein Ar is substituted or unsubstituted aryl or hetereoaryl and X² is halo such as chloride or bromide) can be reacted with various boronates or boronic acids such as Ar²B(OH)₂ (wherein Ar² is substituted or unsubstituted aryl or hetereoaryl) to give a compound of formula 4-2 under Suzuki coupling conditions.

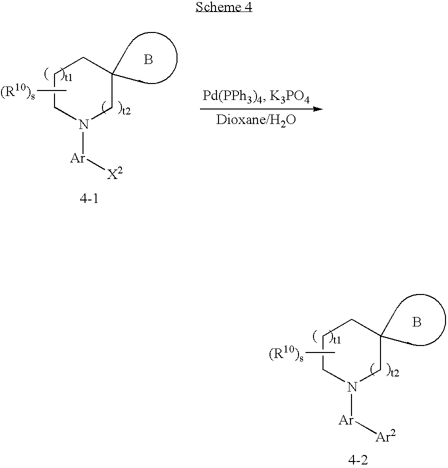

A series of spiro-carbamates of formula 5-6 can be prepared by the method outlined in Scheme 5. The vinyl compound 5-2 (obtained from treating ketone 5-1 with CH₂═PPh₃, generated in situ from methyltriphenylphosphonium bromide in toluene/THF in the present of a base such as LiHMDS) was converted to an epoxide 5-3 by treatment with mCPBA in the presence of sodium carbonate in DCM. Opening of the epoxide ring of the compound 5-3 with an appropriate amine R¹²NH₂ in the presence of LiClO₄ affords an amino-alcohol 5-4 which can be transformed into the compound 5-5 in the presence of C(O)Cl₂ and a suitable base such as triethylamine (TEA). The PG group in Scheme 5 is a nitrogen protecting group. An ordinary skilled in the art would readily recognize/select suitable nitrogen protecting group according to the chemical transformation desired. Examples of suitable nitrogen protecting include benzyl (Bn), carbobenzyloxy (Cbz, i.e., benzyloxycarbonyl) and tert-butyloxycarbonyl (Boc). The nitrogen protecting group (PG) of the compound 5-5 can then be removed by conventional method known to one skilled in the art according to the protecting group used (e.g., hydrogenolysis if PG is Bn or Cbz, or treatment with an acid, such as TFA or HCl, if PG is Boc) to afford the desired compound 5-6.

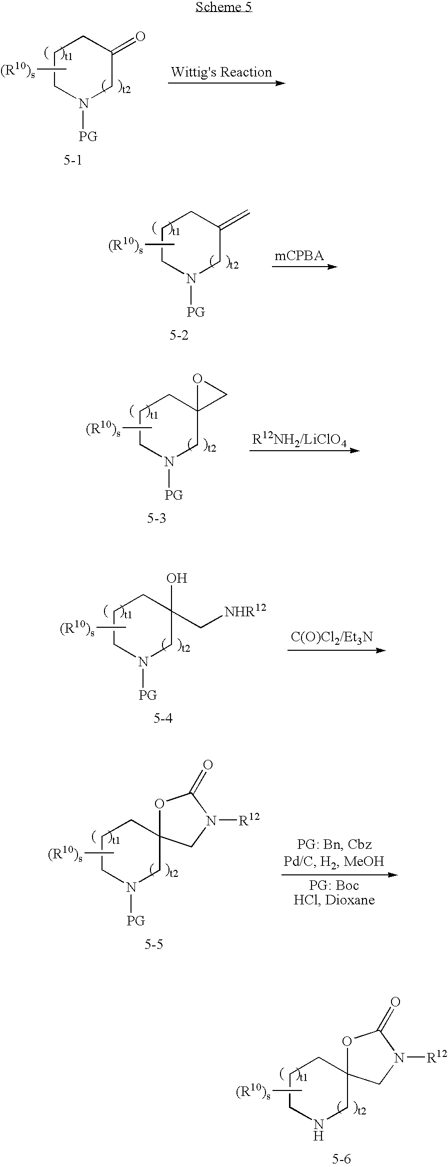

A series of spiro-isoxazolines of formula 6-6 can be prepared according to the procedures outlined in Scheme 6. Reaction of an appropriate aldehyde 6-1 with hydroxylamine hydrochloride in methanol gives an oxime 6-2, which can be converted to an intermediate nitrile oxide 6-3 in situ upon treatment with NCS and a suitable base such as TEA. Reaction of the nitrile oxide 6-3 with an alkene 6-4 (wherein PG is a nitrogen protecting group) yields a protected isoxazoline 6-5, which affords the desired product 6-6 upon removal of the protecting group PG (similar to the method described in Scheme 5).

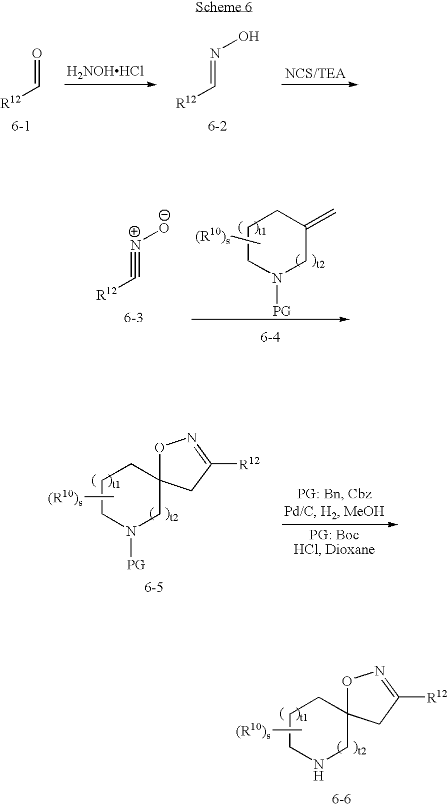

A series of cycloamines 7-5 can be prepared by the method outlined in Scheme 7. A ketone 7-1 (wherein PG is a nitrogen protecting group) can be readily converted to a spirohydantoin 7-2 under Bucherer-Bergs conditions, using, e.g., ammonium carbonate and either sodium cyanide or potassium cyanide in aqueous ethanol. Alkylation of the spirohydantoin 7-2 with one equivalent of R¹³X¹ such as an alkyl halide (wherein R¹³ can be alkyl, cycloalkyl, aryl or the like; and X¹ is a leaving group such as halo) in the presence of a suitable base such as potassium carbonate in a suitable solvent such as DMF, followed by a second alkylation with R¹²X² (wherein R¹² is alkyl, cycloalkyl, aryl or the like; and X² is a leaving group such as halo) in the presence of a suitable base such as sodium hydride in a suitable solvent such as DMF provides a substituted hydantoin 7-3. Reduction of the amide carbonyl using LiAlH₄/AlCl₃ in THF gives a spiro-urea 7-4 (see, e.g., Reichard, G. A. et. al. Org. Lett. 2003, 5, 4249), which upon removal of the protecting group PG yields the desired cycloamine or a salt thereof 7-5.

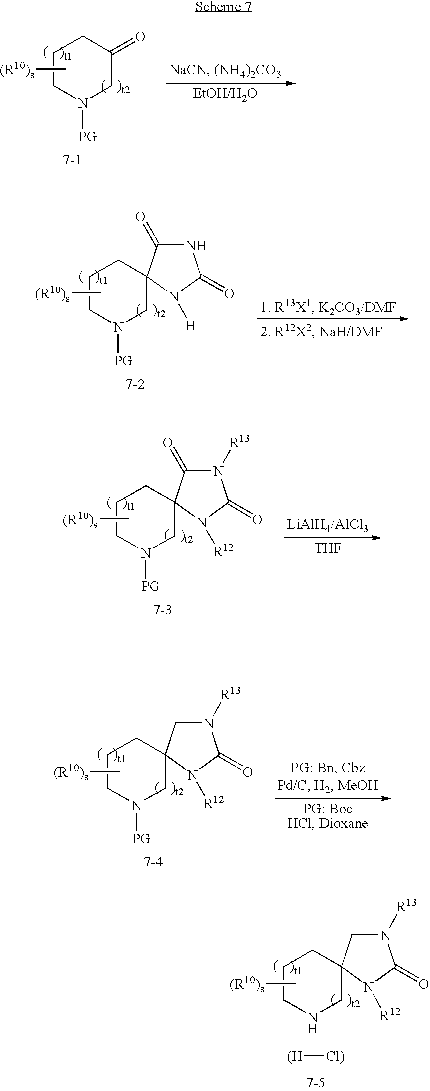

As shown in Scheme 8, treatment of a ketone 8-1 (wherein PG is a nitrogen protecting group) with an amine R¹²NH₂ (wherein R¹² is alkyl, cylcoalkyl or the like) and sodium cyanide can provide a hydantoin derivative 8-2. Aromatic substituted hydantoins 8-4 can be obtained by coupling the hydantoin derivative 8-2 with an aromatic boronic acid or aromatic halide 8-3 (wherein X² is halo such as bromo or chloro; and each R is independently selected from alkyl, OH, alkoxy, haloalkyl and the like; and m5 is 0, 1, 2, 3, 4, or 5) in the presence of a suitable catalyst such as a palladium catalyst. LiAlH₄/AlCl₃ reduction of the amide carbonyl group and subsequent removal of the nitrogen protecting group PG yield the desired spiro-urea 8-6.

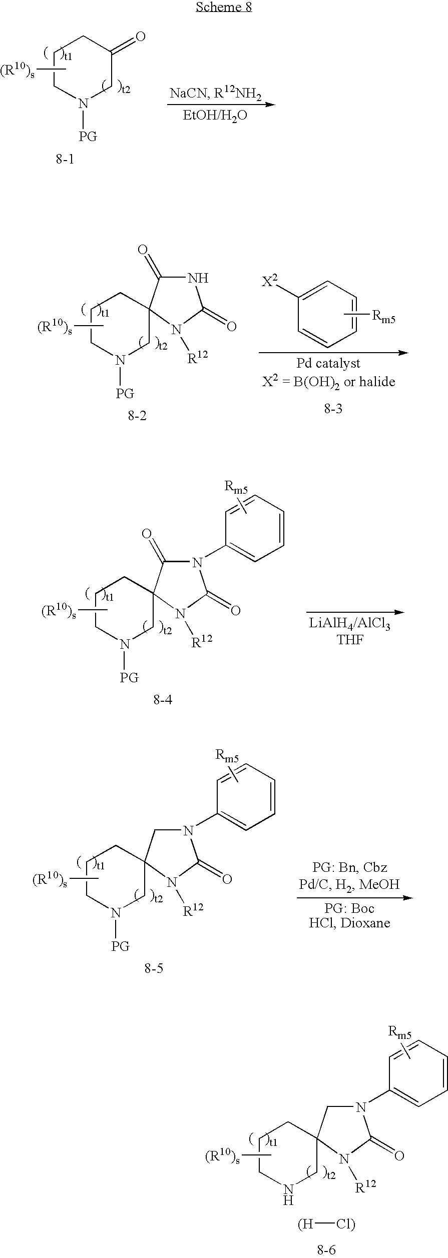

In an alternative route, spiro-urea 9-6 (wherein R^{12′} is, e.g., alkyl, aryl, heteroaryl, cycloalkyl, heterocycloalkyl, arylalkyl or the like) can be prepared by the method outlined in Scheme 9. The protected amino acid 9-1 can be coupled with an amine R¹³NH₂ by conventional methods such as using a coupling reagent for amide bond formation such as BOP to provide an amide 9-2 which, in turn, can be subject to hydrogenolysis in the presence of Pd catalyst to yield an amine 9-3. The reductive amination of the amine 9-3 with a suitable aldehyde R^{12′} CHO gives an amide 9-4. Reduction of the carbonyl group of the amide 9-4 using LAH in THF gives the di-amine 9-5, which can be converted to the desired spiro-urea 9-6 upon treatment with oxalyl chloride in dichloromethane (DCM) in the presence of a suitable base such as triethylamine (TEA) or 4,5-dicyanoimidazole (DCI), followed by acidic cleavage of the Boc group.

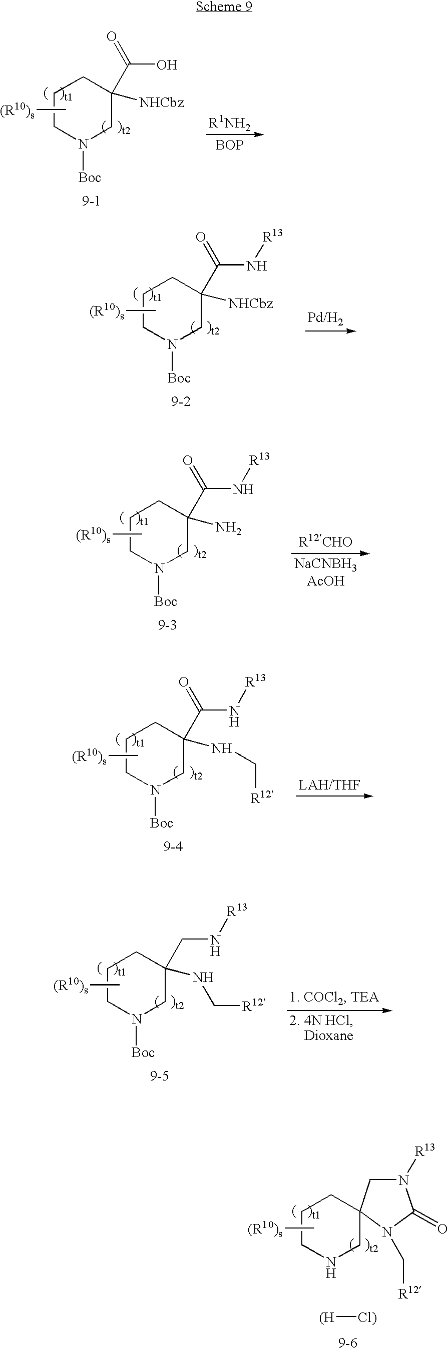

A series of spiro-lactams of formula 10-3 (wherein R^{12′} is, e.g., alkyl, aryl, heteroaryl, cycloalkyl, heterocycloalkyl, arylalkyl or the like) can be prepared by the method outlined in Scheme 10. A spiro-lactam 10-3 can be obtained from an amino-amide 10-1 by treatment with formaldehyde in toluene in the presence of acid catalyst such as p-TsOH, followed by removal of the Boc group under acid conditions.

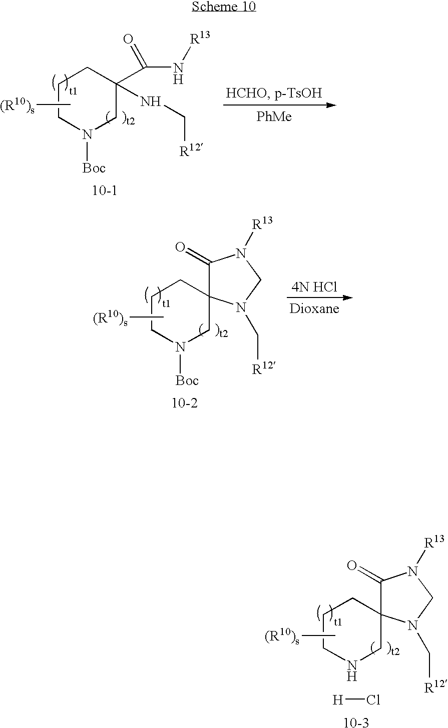

A series of spiro-sulfamides of formula 11-3 can be prepared according to the method outlined in Scheme 11. A diamine 11-1 can be treated with S(O)ₚ₁Cl₂ (wherein p1 is 1 or 2) in a suitable base such as DCM and in the presence of base such as Hunig's base to give the Boc-protected spiro-sulfamide 11-2, which yields the desired spiro-sulfamide 11-3 upon removal the Boc group under acid conditions.

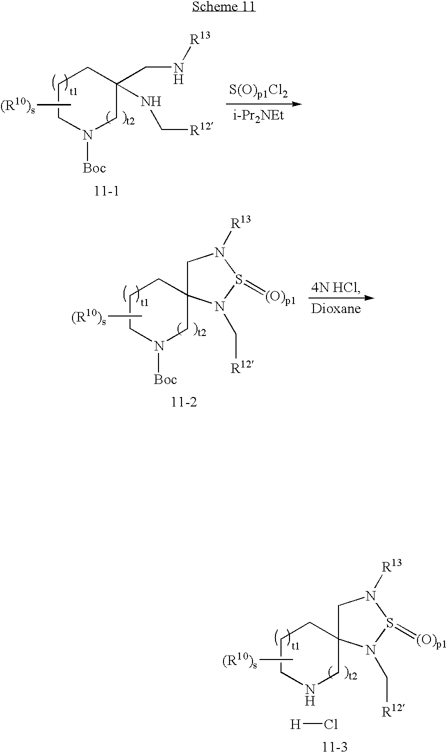

Spiro-sulfamides of formula 12-5 can be prepared by the methods outlined in Scheme 12. Strecker reaction followed by LAH reduction starting with a ketone 12-1 (wherein PG is a suitable nitrogen protecting group such as Boc or Bn) can give a diamine 12-2. Cyclization of the diamine 12-2 with sulfamide in pyridine yields a spiro-sulfamide 12-3. Alkylation of the spiro-sulfamide 12-3 with one equivalent of R¹³X¹ such as an alkyl halide (wherein R¹³ is alkyl, cycloalkyl, aryl or the like; and X¹ is a leaving group such as halo) in the presence of a suitable base such as sodium hydride in a suitable solvent such as DMF, followed by a second alkylation with R¹²X² (wherein R¹² is alkyl, cycloalkyl, aryl or the like; and X² is a leaving group such as halo) in the presence of a suitable base such as sodium hydride in a suitable solvent such as DMF provides a substituted spiro-sulfamide 12-4. Removal of the protecting group PG of compound 12-4 as previously described produces the desired spiro-sulfamide 12-5.

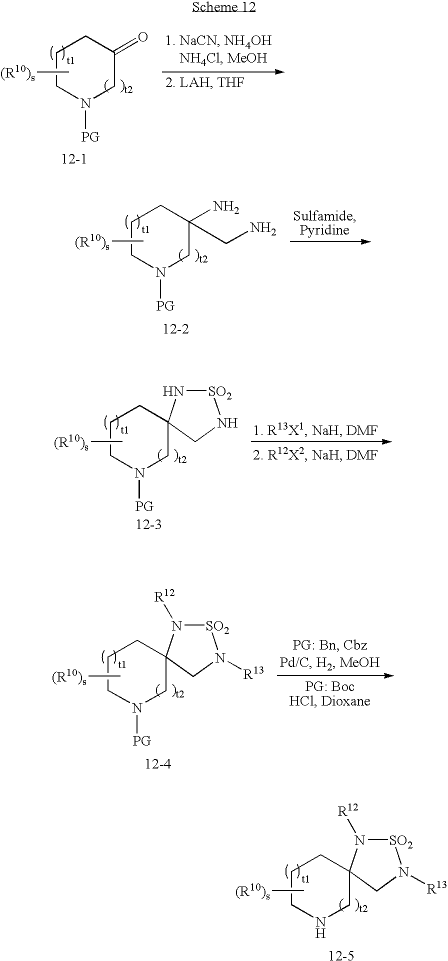

In a similar manner, a series of spiro-sulfamides of formula 13-5 can be prepared by the methods outline in Scheme 13. Aromatic substituted spiro-sulfamides 13-5 can be obtained by coupling a compound 13-3 with an aromatic boronic acid or aromatic halide 13-4 (wherein X² is halo such as bromo or chloro; each R is independently selected from alkyl, OH, alkoxy, haloalkyl and the like; and m5 is 0, 1, 2, 3, 4, or 5) in the presence of a suitable catalyst such as a palladium catalyst. Removal of the nitrogen protecting group PG (e.g., Boc) of compound 13-5 yields the desired spiro-sulfamides 13-6.

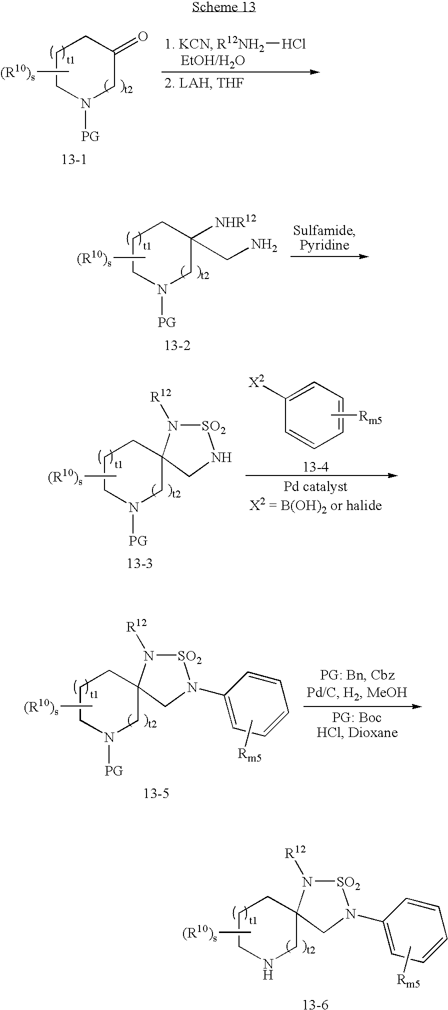

A series of spiro-sulfonamides of formula 14-6 can be prepared according to the method outlined in Scheme 14. A sulfonyl chloride 14-1 can be reacted with a primary amine R¹²NH₂ (wherein R¹² is selected from alkyl, arylalkyl and the like) to afford a compound 14-2. Intra-molecular N-alkylation of the compound 14-2 affords a cyclo-sulfoamide 14-3 which is then converted to the spiro-sulfonamide 14-5 by coupling to a compound 14-4 having two leaving groups such as a dibromo, dichloro or bissulonate derivative (wherein Lg¹ and Lg² are independently selected from bromo, chloro, and the like; and PG is a nitrogen protecting group) in a suitable solvent such as THF and in the presence of a suitable base such as LiHMDS. Removal of the protecting group PG of compound 14-5 affords of the desired spiro-sulfonamide 14-6.

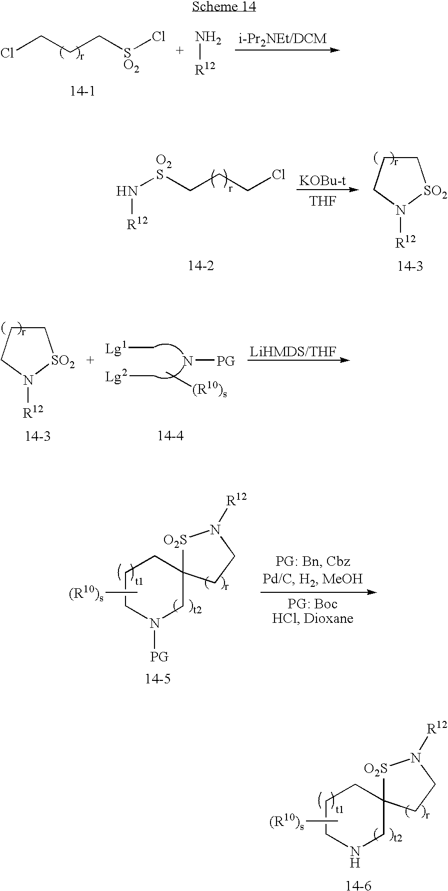

A series of spiro-sulfonamides of formula 15-9 can be prepared according to the method outlined in Scheme 15. A thioacetate 15-3 can be prepared from the intermediate iodide compound 15-2 which is generated in situ by addition of a suitable base such as LDA to an acid ester 15-1 (wherein R is alkyl, aryl, arylalkyl or the like; and PG is a nitrogen protecting group) followed by an addition of diiodomethane. Oxidation of the thioacetate 15-3 to the sulfonyl chloride 15-4 can be achieved by using chlorine gas in dichloromethane (DCM) and water. Sulfonyl chloride 15-4 is then converted to the cyclic sulfonamide 15-5 by treatment with a primary amine R¹²NH₂ in the presence of a suitable base such Hunig's base or DIPEA at 0° C. followed by heating the mixture to 80° C. LiAlH₄/AlCl₃ reduction of the carbonyl group of the compound 15-5 followed by removal of the protecting group PG gives the desired spiro-sulfonamide 15-7.

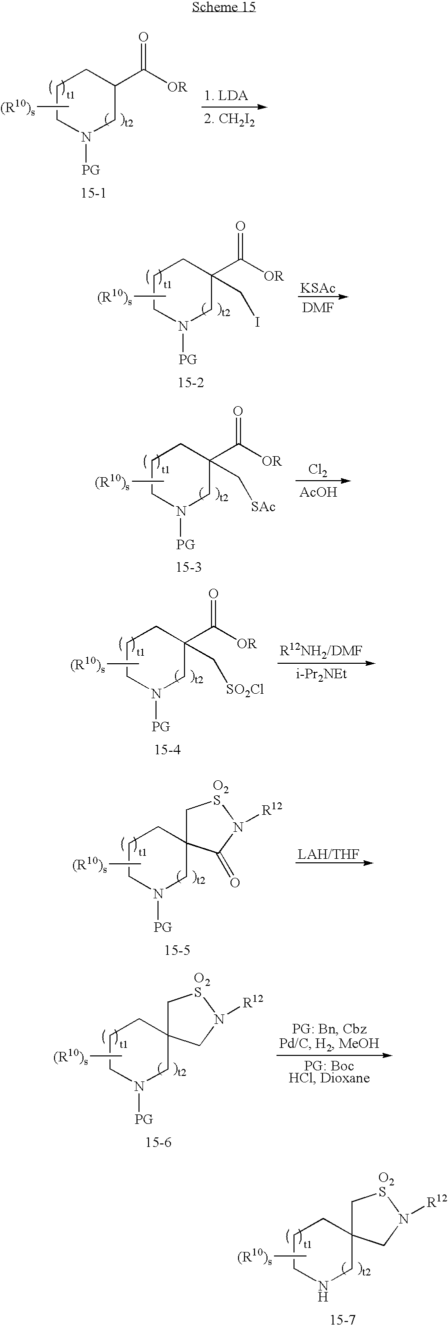

A series of amides of formula 16-4 and/or 16-5 can be prepared according to the method outlined in Scheme 16. Coupling of an acid 16-1 with an amine HNR⁵R⁶ forms an amide 16-2, of which the oxo group on the ring can be reduced to OH group (thus generating an alcohol 16-3) by using a suitable reducing reagent such as sodium borohydride in methanol. Mitsunobu reaction of 16-3 with ArOH (wherein Ar is optionally substituted aryl or an optionally substituted heteroaryl) yields the desired ether product 16-4. Alternatively, O-alkylation of compound 16-3 with ArCH₂X¹ (wherein Ar is an optionally substituted aryl or an optionally substituted heteroaryl; and X¹ is a leaving group such as halo) in the presence of a suitable base such as sodium hydride in DMF gives the ether product 16-5.

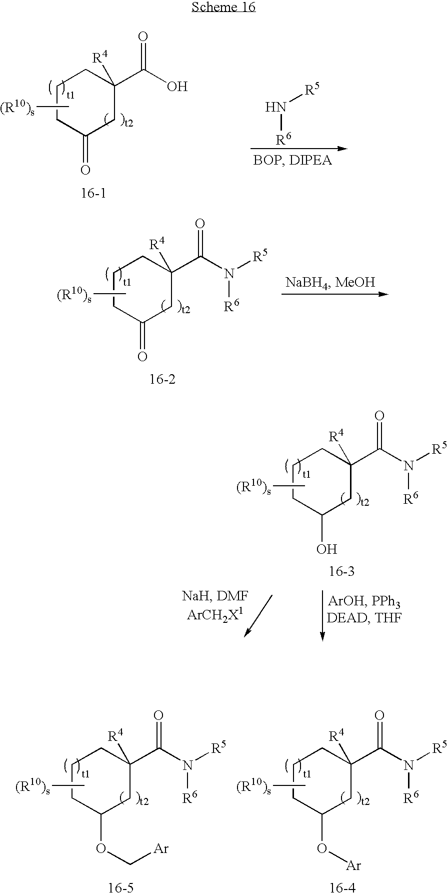

A series of thio-ethers of formula 17-3 and sulfones of formula 17-4 can be prepared by the methods outlined in Scheme 17. Conversion of the OH group of 17-1 to the mesylate group of 17-2 can be achieved by using methylsulfonyl chloride in the presence of a base such as Hunig's base, triethylamine or DBU, and in a solvent such as DCM, THF, or dioxane. Reaction of 17-2 with a thio-compound of ArSH (wherein Ar is optionally substituted aryl or an optionally substituted heteroaryl) affords a thio-ether 17-3 which can be oxidized to an sulfone 17-4 by using a suitable oxidant such as 2KHSO₅.KHSO₄.K₂SO₄ (the active ingredient of which is potassium peroxymonosulfate) which is available under the trade mark OXONE®, under suitable conditions.

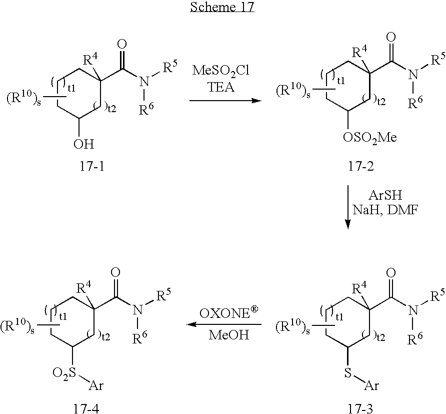

A series of amides of formulas 18-2, 18-3 and 18-4 can be conveniently prepared according to the methods outlined in Scheme 18. Reaction of a keto-amide 18-1 with a Grignard reagent ArMgBr (wherein Ar is optionally substituted aryl or an optionally substituted heteroaryl) in a suitable solvent such as THF or diethylether will afford an alcohol-amide 18-2. Treatment of the alcohol-amide 18-2 with TFA produces the alkene-amide 18-3, which can be reduced to an amide 18-4 by hydrogenation such as catalytic hydrogenation (e.g., using palladium on carbon) in a suitable solvent such as methanol.

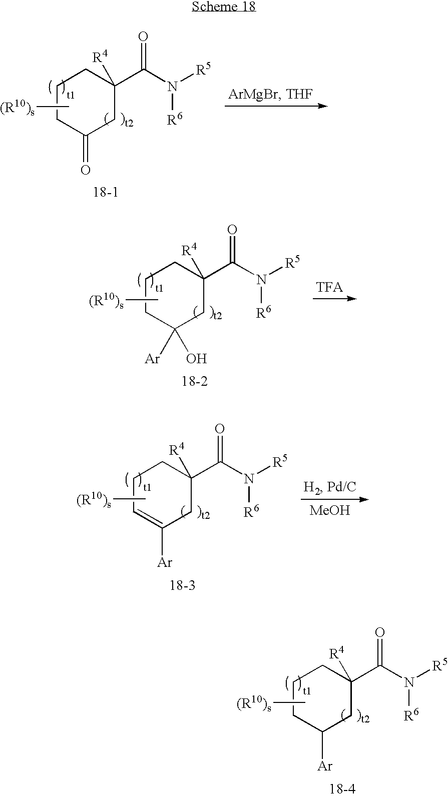

As shown in Scheme 19, a series of amides of formulas 19-5 and 19-6 can be prepared from a keto-ester 19-1 (wherein R is alkyl, aryl, arylalkyl or the like). Reaction of the keto-ester 19-1 with a Grignard reagent ArMgBr (wherein Ar is optionally substituted aryl or an optionally substituted heteroaryl) in a suitable solvent such as THF or diethylether will afford an alcohol 19-2, which upon treatment with TFA produces an alkene 19-3. The ester group of the compound 19-3 can be hydrolyzed (e.g. under a basic condition) and the resulting acid 19-4 can be coupled with amine HNR⁵R⁶ to afford the amide 19-5 using a conventional amide formation method (e.g., using a coupling reagent such as BOP, and in the presence of a suitable base such as TEA or DIPEA). The alkene group of the amide 19-5 can be reduced by hydrogenation such as catalytic hydrogenation (e.g., using palladium on carbon) in a suitable solvent such as methanol to afford the amide 19-6.

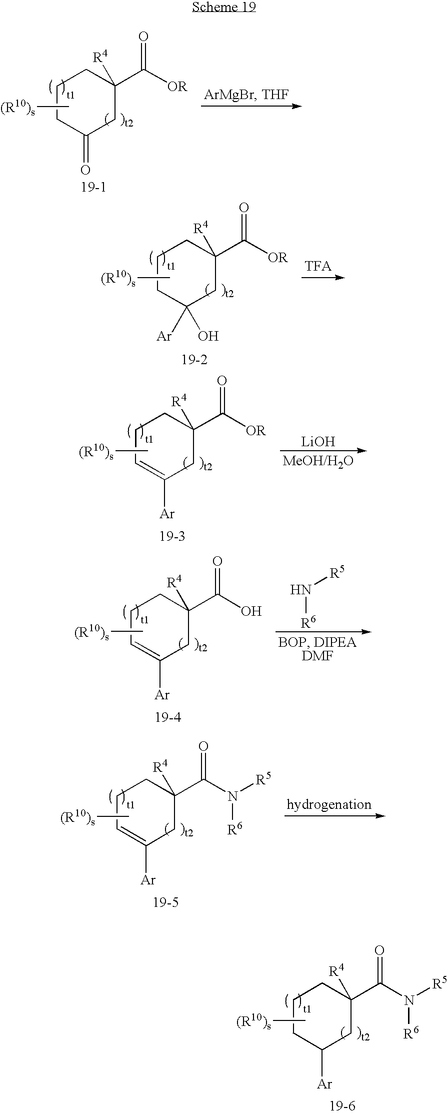

A series of amides of formulas 20-2 and 20-3 can be prepared by the methods outlined in Scheme 20. Wittig reaction of a keto-amide 20-1 with R^{L}CH═PPh₃ in toluene gives an amide 20-2. Alternatively, the amide 20-2 can be obtained from an keto-ester 20-4. The keto-ester 20-4 can be reacted with R^{L}CH═PPh₃ (Wittig reaction) to afford the ester 20-5, which upon hydrolysis can afford the acid 20-6. The acid 20-6 can be coupled with amine HNR⁵R⁶ to afford the amide 20-2 using a conventional amide formation method (e.g., using a coupling reagent such as BOP, and in the presence of a suitable base such as TEA or DIPEA). The alkene group of the amide 20-2 can be reduced by hydrogenation such as catalytic hydrogenation (e.g., using palladium on carbon) in a suitable solvent such as methanol to afford the amide 20-3. 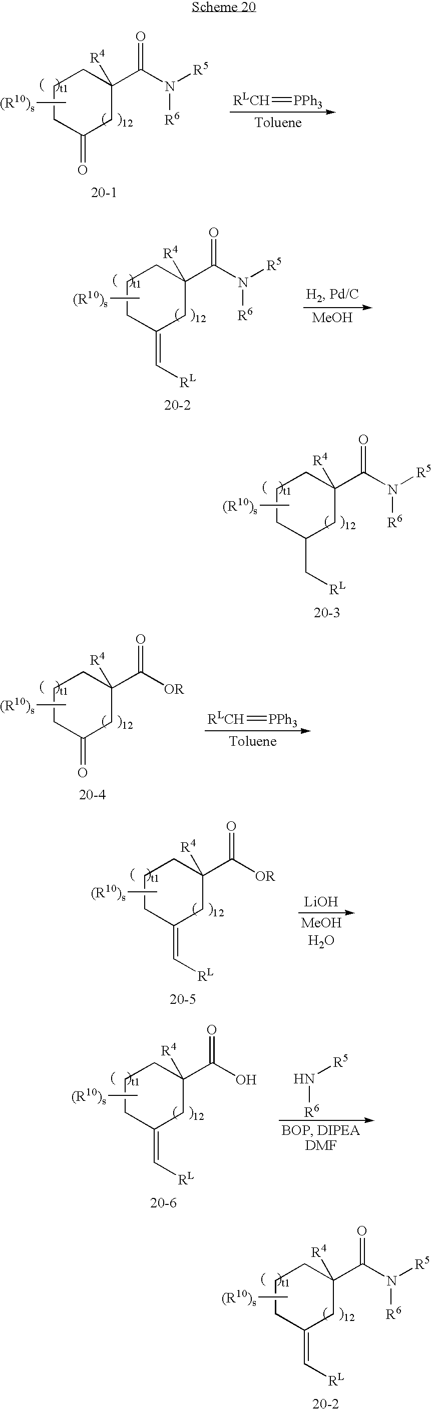
Methods

Compounds of the invention can modulate activity of 11βHSD1. The term “modulate” is meant to refer to an ability to increase or decrease activity of an enzyme. Accordingly, compounds of the invention can be used in methods of modulating 11βHSD1 by contacting the enzyme with any one or more of the compounds or compositions described herein. In some embodiments, compounds of the present invention can act as inhibitors of 11βHSD1. In further embodiments, the compounds of the invention can be used to modulate activity of 11βHSD1 in an individual in need of modulation of the enzyme by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme or receptor. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, atherosclerosis, hypertension, hyperlipidemia, cognitive impairment, dementia, depression (e.g., psychotic depression), glaucoma, cardiovascular disorders, osteoporosis, and inflammation. Further examples of 11βHSD1-associated diseases include metabolic syndrome, coronary heart disease, type 2 diabetes, hypercortisolemia, androgen excess (hirsutism, menstrual irregularity, hyperandrogenism) and polycystic ovary syndrome (PCOS).

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder; and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, for example see International Patent Application No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to labeled compounds of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br or ⁷⁷Br will generally be most useful.

It is understood that a “radio-labeled compound” is a compound that has incorporated at least one radionuclide. In some embodiments the radionuclide is selected from ³H, ¹⁴C, ¹²⁵I, ³⁵S and ⁸²Br.

In some embodiments, the labeled compounds of the present invention contain a fluorescent lable.

Synthetic methods for incorporating radio-isotopes and fluorescent labels into organic compounds are are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind a 11βHSD1 by monitering its concentration variation when contacting with the 11βHSD1, through tracking the labeling. For another example, a test compound (labeled) can be evaluated for its ability to reduce binding of another compound which is known to bind to 11βHSD1 (i.e., standard compound). Accordingly, the ability of a test compound to compete with the standard compound for binding to the 11βHSD1 directly correlates to its binding affinity. Conversely, in some other screening assays, the standard compound is labled and test compounds are unlabeled. Accordingly, the concentration of the labled standard compound is monitored in order to evaluate the competition between the standard compound and the test compound, and the relative binding affinity of the test compound is thus ascertained.

Kits

The present invention also includes pharmaceutical kits useful, for example, in the treatment or prevention of 11βHSD1-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. Certain compounds of the Examples were found to be inhibitors of 11βHSD1 according to one or more of the assays provided herein.

### EXAMPLES

Preparations for compounds of the invention are provided below. In some instances, the crude product was a mixture of regioisomers. Typically, these isomers were separated on a preparative scale by HPLC or flash chromatography (silica gel) as indicated in each of the Examples. Typical preparative RP-HPLC column conditions were as follows:

pH=2 purifications: Waters Sunfire™ C₁₈ 5 μm, 19×100 mm column, eluting with mobile phase A: 0.1% TFA (trifluoroacetic acid) in water and mobile phase B: 0.1% TFA in acetonitrile; the flow rate was 30 mL/m, the separating gradient was optimized for each compound using the Compound Specific Method Optimization protocol as described in literature [“Preparative LC-MS Purification: Improved Compound Specific Method Optimization”, K. Blom, B. Glass, R. Sparks, A. Combs, J. Combi. Chem., 6, 874-883 (2004)].

pH=10 purifications: Waters XBridge C₁₈ 5 μm, 19×100 mm column, eluting with mobile phase A: 0.15% NH₄OH in water and mobile phase B: 0.15% NH₄OH in acetonitrile; the flow rate was 30 mL/m, the separating gradient was optimized for each compound using the Compound Specific Method Optimization protocol as described in literature [“Preparative LC-MS Purification: Improved Compound Specific Method Optimization”, K. Blom, B. Glass, R. Sparks, A. Combs, J. Combi. Chem., 6, 874-883 (2004)].

The separated isomers were then typically subjected to analytical LC/MS for purity check under the following conditions: Instrument; Agilent 1100 series, LC/MSD, Column: Waters Sunfire™ C₁₈ 5 μm, 2.1×5.0 mm, Buffers: mobile phase A: 0.025% TFA in water and mobile phase B: 0.025% TFA in acetonitrile; gradient 2% to 80% of B in 3 min with flow rate 1.5 mL/min. Retention time (Rt) data in the Examples refer to these analytical LC/MS conditions unless otherwise specified.

### Example 1

### 7-Acetyl-3-[1-(4-chlorophenyl)cyclopropyl]-1-oxa-2,7-diazaspiro[4.4]non-2-ene

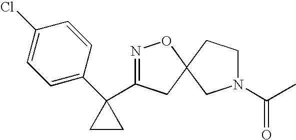

### Step1 . [1-(4-chlorophenyl)cyclopropyl]methanol

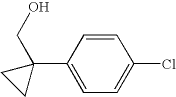

A solution of borane in tetrahydrofuran (1.0 M, 60 ml, 60 mmol) was added to a solution of 1-(4-chlorophenyl)cyclopropanecarboxylic acid (7.95 g, 40.4 mmol) in tetrahydrofuran (50 mL) at 0° C., and the resulting solution was stirred at 0° C. for 1 h. The solvent was evaporated under reduced pressure. The residue was co-evaporated (azeotroped) with methanol (3×10 mL) to afford [1-(4-chlorophenyl)cyclopropyl]methanol (6.7 g, 0.037 mol).

### Step 2. 1-(4-chlorophenyl)cyclopropanecarbaldehyde

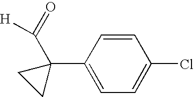

With stirring, to a solution of [1-(4-chlorophenyl)cyclopropyl]methanol (7.20 g, 0.0394 mol) in acetone (100 mL) was added a solution of chromium(VI) oxide (4.14 g, 0.0414 mol) in water (12.0 mL) and sulfuric acid (3.49 mL, 0.0642 mol) over 15 min in the presence of an ice-water bath. The mixture was stirred at 0° C. for 1 h, and then iso-propanol (10 mL) was added. The mixture was stirred for an additional 5 min, and filtered through a pad of silica gel. The filtrate was concentrated. The residue was purified by Combiflash with ethylacetate/hexane (20%) to afford 1-(4-chlorophenyl)cyclopropanecarbaldehyde (3.20 g, 0.225 mol).

### Step 3. 1-(4-chlorophenyl)cyclopropanecarbaldehyde oxime

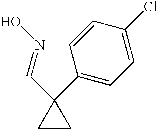

A mixture of 1-(4-chlorophenyl)cyclopropanecarbaldehyde (2.70 g, 0.0149 mol) and hydroxylamine hydrochloride (2.35 g, 0.0338 mol) in methanol (50 mL, 1 mol) was heated to reflux for 4 hours. The solvent was distilled under reduced pressure. The residue was treated with ether and filtered. The filtrate was concentrated. The residue was purified by Combiflash with Ethyl acetate/hexane (20%) to give 1-(4-chlorophenyl)cyclopropanecarbaldehyde oxime (1.53 g, 0.00428 mol)

### Step 4. tert-butyl 3-methylenepyrrolidine-1-carboxylate

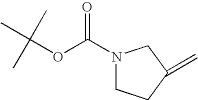

To methyl(triphenyl)phosphorane hydrobromide (1.43 g, 0.00400 mol) in toluene (12.0 mL) was added sodium hexamethyldisilazane in tetrahydrofuran (1.00 M, 4.00 mL). The mixture was stirred at rt for 2 h. The solution was added to a solution of tert-butyl 3-oxopyrrolidine-1-carboxylate (0.74 g, 0.0040 mol) in toluene (10 mL) at 0° C. over a period of 5 min. The ice bath was removed and the mixture was allowed to warm up to room temperature (RT). The mixture was then stirred at RT for overnight. The reaction mixture was diluted with hexane (30 mL), and filtered through a pad of silica gel and washed with hexane to give tert-butyl 3-methylenepyrrolidine-1-carboxylate (650 mg, 88.6%).

### Step 5. tert-butyl 3-[1-(4-chlorophenyl)cyclopropyl]-1-oxa-2,7-diazaspiro[4.4]non-2-ene-7-carboxylate

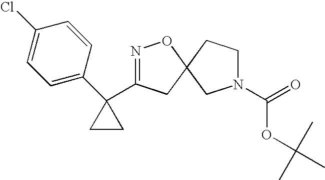

To a solution of 1-(4-chlorophenyl)cyclopropanecarbaldehyde oxime (19.57 mg, 0.0001000 mol) in DMF (0.3 mL) was added N-chlorosuccinimide (13.4 mg, 0.000100 mol) in DMF (0.100 mL). The mixture was stirred at RT for 3 h. Triethylamine (20.0 uL) was added. The mixture was stirred for 5 min and then tert-butyl 3-methylenepyrrolidine-1-carboxylate (22.0 mg, 0.000120 mol) was added. The mixture was stirred at RT for overnight. The mixture was diluted with methanol (1.0 mL) and adjusted to be acidic with TFA (pH˜2.0). The resulting mixture was filtered. The filtrated was purified by prep-HPLC to give tert-butyl 3-[1-(4-chlorophenyl)cyclopropyl]-1-oxa-2,7-diazaspiro[4.4]non-2-ene-7-carboxylate (20 mg, 52%).

### Step 6. 3-[1-(4-chlorophenyl)cyclopropyl]-1-oxa-2,7-diazaspiro[4.4]non-2-ene hydrochloride

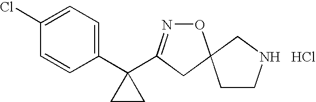

Hydrogen chloride in 1,4-dioxane (4.0 M, 0.50 mL) was added to tert-butyl 3-[1-(4-chlorophenyl)cyclopropyl]-1-oxa-2,7-diazaspiro[4.4]non-2-ene-7-carboxylate (15.0 mg, 0.0000398 mol) at rt. The mixture was stirred at RT for 1 h. The solvent was evaporated. The residue was dried under reduced pressure to afford 3-[1-(4-chlorophenyl)cyclopropyl]-1-oxa-2,7-diazaspiro[4.4]non-2-ene hydrochloride (12.4 mg, 99.5%).

### Step 7. 7-acetyl-3-[1-(4-chlorophenyl)cyclopropyl]-1-oxa-2,7-diazaspiro[4.4]non-2-ene

N,N-Diisopropylethylamine (20.0 μL, 0.000115 mol) was added to 3-[1-(4-chlorophenyl)cyclopropyl]-1-oxa-2,7-diazaspiro[4.4]non-2-ene hydrochloride (10.5 mg, 0.0000335 mol) in acetonitrile (1.0 mL). To the solution was added acetyl chloride (6.0 μL, 0.000084 mol). The mixture was stirred at RT for overnight, and was diluted with methanol (1 ml). The resulting solution was purified by prep-HPLC (pH=2 conditions) to give a TFA salt of 7-acetyl-3-[1-(4-chlorophenyl)cyclopropyl]-1-oxa-2,7-diazaspiro[4.4]non-2-ene (5.6 mg). LCMS: (M+H)⁺=319.1/321.1.

### Example 2

### Methyl (8S)-7-acetyl-3-[1-(4-chlorophenyl)cyclopropyl]-1-oxa-2,7-diazaspiro[4.4]non-2-ene-8-carboxylate

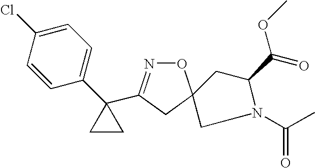

### Step 1. 1-tert-butyl 2-methyl (2S)-4-oxopyrrolidine-1,2-dicarboxylate

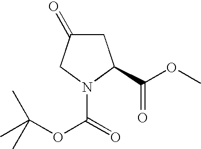

With stirring, to a solution of mthyl (2S,4R)-N-tert-butoxycarbonyl-4-hydroxy-2-pyrrolidinecarboxylate (2.00 g, 0.00815 mol) in acetone (50.0 mL) and ether (50 mL) was added a solution of chromium(VI) oxide (1.90 g, 0.0190 mol) in water (5.50 mL) and sulfuric acid (1.60 mL, 0.0294 mol) over 15 min in the presence of an ice-water bath. The ice-water bath was removed and the mixture was stirred at RT for 30 min and then iso-propanol (10 mL) was added. The mixture was stirred for an additional 5 min. The mixture was filtered through a pad of silica gel plus potassium carbonate. The filtrated was concentrated. The residue was purified by flash chromatography with ethyl acetate/heaxane (25%) to give the desired product (1.12 g).

### Step 2. 1-tert-butyl 2-methyl (2S)-4-methylenepyrrolidine-1,2-dicarboxylate

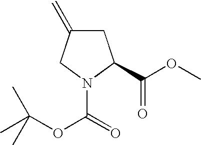

To solution of methyl(triphenyl)phosphorane hydrobromide (1.79 g, 0.00500 mol) in toluene (15.0 mL) was added a solution of sodium hexamethyldisilazane in tetrahydrofuran (1.00 M, 5.00 mL). The mixture was stirred at RT for 2 h. The mixture was added to a solution of 1-tert-butyl 2-methyl (2S)-4-oxopyrrolidine-1,2-dicarboxylate (1.00 g, 0.00411 mol) in toluene (10 mL) at −20° C. over a period of 5 min. The cold bath was removed, and the mixture was allowed to warm to RT. The mixture was stirred at RT for additional 3 h, diluted with ethyl acetate (30 mL) and washed with brine (3×5 mL). The organic layer was dried (over Na₂SO₄), filtered, concentrated under reduced pressure. The residue was purified by Combiflash with ethyl acetate/heaxane (25%) to give the desired product (270 mg, 27%).

### Step 3. 7-tert-butyl 8-methyl (8S)-3-[1-(4-chlorophenyl)cyclopropyl]-1-oxa-2,7-diazaspiro[4.4]non-2-ene-7,8-dicarboxylate

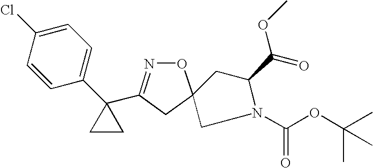

To a solution of 1-(4-chlorophenyl)cyclopropanecarbaldehyde oxime (78.2 mg, 0.000400 mol) in DMF(1.0 mL) was added N-chlorosuccinimide (53.4 mg, 0.000400 mol) in DMF (0.4 mL). The mixture was stirred at RT for 3 h and then triethylamine (40.5 mg, 0.000400 mol) in DMF (0.4 mL) was added. The mixture was stirred for 5 min and then 1-tert-butyl 2-methyl (2S)-4-methylenepyrrolidine-1,2-dicarboxylate (96.5 mg, 0.000400 mol) in DMF (0.4 mL) was added. The mixture was stirred at RT for overnight. The reaction mixture was diluted with ethyl acetate (5 mL), and washed with water (2×2 mL). The organic layer was dried over Na₂SO₄ and concentrated. The residue was dissolved in DMF and was adjusted to be acidic with TFA (pH˜2.0). The resulting solution was purified by prep-HPLC to give 7-tert-butyl 8-methyl (8 S)-3-[1-(4-chlorophenyl)cyclopropyl]-1-oxa-2,7-diazaspiro[4.4]non-2-ene-7,8-dicarboxylate (68 mg, 39%).

### Step 4. Methyl (8S)-3-[1-(4-chlorophenyl)cyclopropyl]-1-oxa-2,7-diazaspiro[4.4]non-2-ene-8-carboxylate hydrochloride

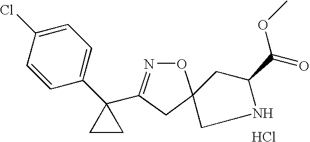

7-tert-Butyl 8-methyl (5S,8S)-3-[1-(4-chlorophenyl)cyclopropyl]-1-oxa-2,7-diazaspiro[4.4]non-2-ene-7,8-dicarboxylate (61.0 mg, 0.000140 mol) in a solution of hydrogen chloride in 1,4-dioxane (4.0 M, 1.0 mL) was stirred at RT for 1 h. The solvent was evaporated under reduced pressure. The residue was dried under high vacuum to give methyl (8S)-3-[1-(4-chlorophenyl)cyclopropyl]-1-oxa-2,7-diazaspiro[4.4]non-2-ene-8-carboxylate hydrochloride (50 mg).

### Step 5. Methyl (8S)-7-acetyl-3-[1-(4-chlorophenyl)cyclopropyl]-1-oxa-2,7-diazaspiro[4.4]non-2-ene-8-carboxylate

N,N-Diisopropylethylamine (20.0 μL 0.000115 mol) was added to a solution of methyl (5S,8S)-3-[1-(4-chlorophenyl)cyclopropyl]-1-oxa-2,7-diazaspiro[4.4]non-2-ene-8-carboxylate (12.5 mg, 0.0000373 mol) in acetonitrile (1.0 mL), followed by acetyl chloride (5.0 μL, 0.000070 mol). The mixture was stirred at RT for overnight, and diluted with methanol to 2.0 ml and was adjusted to be acidic with TFA (pH˜2.0). The resulting solution was purified by prep-HPLC (pH=2 conditions) to give methyl (8S)-7-acetyl-3-[1-(4-chlorophenyl)cyclopropyl]-1-oxa-2,7-diazaspiro[4.4]non-2-ene-8-carboxylate (8.6, 61%). LCMS: (M+H)⁺=377.1/379.1.

### Example 3

### Methyl (8S)-3-[1-(4-chlorophenyl)cyclopropyl]-7-(methylsulfonyl)-1-oxa-2,7-diazaspiro[4.4]non-2-ene-8-carboxylate

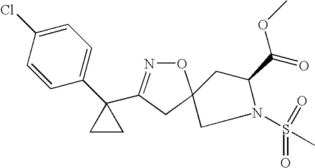

This compound was prepared using procedures analogous to those for example 2. LCMS: (M+H)⁺=413.0/415.1.

### Example 4

### Dimethyl (8S)-3-[1-(4-chlorophenyl)cyclopropyl]-1-oxa-2,7-diazaspiro[4.4]non-2-ene-7,8-dicarboxylate

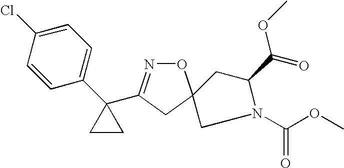

This compound was prepared using procedures analogous to those for example 2. LCMS: (M+H)⁺=393.1/395.1.

### Example 5

### 8-Acetyl-3-[1-(4-chlorophenyl)cyclopropyl]-1-oxa-2,8-diazaspiro[4.5]dec-2-ene

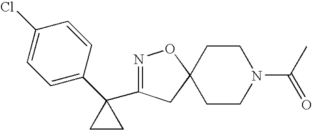

This compound was prepared using procedures analogous to those for example 1. LCMS: (M+H)⁺=333.1/335.1.

### Example 6

### 3-[1-(4-Chlorophenyl)cyclopropyl]-8-(methylsulfonyl)-1-oxa-2,8-diazaspiro[4.5]dec-2-ene

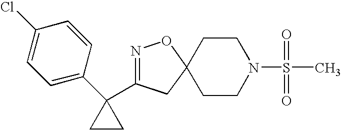

This compound was prepared using procedures analogous to those for example 1. LCMS: (M+H)⁺⁼369.1/371.0.

### Example 7

### Methyl 3-cyclohexyl-1-oxa-2,7-diazaspiro[4.5]dec-2-ene-7-carboxylate

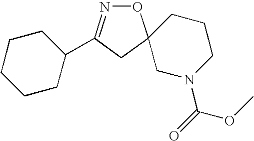

This compound was prepared using procedures analogous to those for example 1. LCMS: (M+H)⁺=281.1.

### Example 8

### 7-[(3-Chloro-2-methylphenyl)sulfonyl]-3-cyclohexyl-1-oxa-2,7-diazaspiro[4.5]dec-2-ene

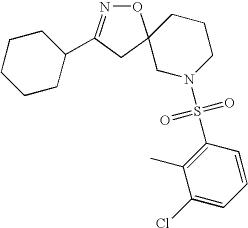

This compound was prepared using procedures analogous to those for example 1. LCMS: (M+H)⁺=411.1/413.1.

### Example 9

### 3-[1-(4-chlorophenyl)cyclopropyl]-8-phenyl-1-oxa-2-azaspiro[4.5]dec-2-ene

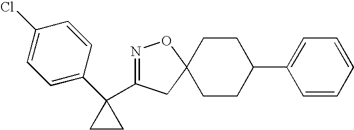

This compound was prepared using procedures analogous to those for example 1. LCMS: (M+H)⁺=366.1/368.1.

### Example 10

### 1′-[(3-chloro-2-methylphenyl)sulfonyl]spiro[indole-3,4′-piperidin]-2(1H)-one

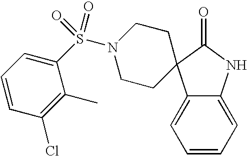

N,N-Diisopropylethylamine (20.0 uL, 0.115 mmol) was added to a solution of spiro[indole-3,4′-piperidin]-2(1H)-one hydrochloride (11.9 mg, 0.050 mmol) in acetonitrile (1.0 mL). To the solution was added 3-chloro-2-methylbenzenesulfonyl chloride (11.2 mg, 0.050 mmol). The mixture was stirred at RT for overnight, and was diluted with methanol (1 ml). The resulting solution was purified by prep-HPLC (pH=2 conditions) to give 1′-[(3-chloro-2-methylphenyl)sulfonyl]spiro[indole-3,4′-piperidin]-2(1H)-one (16.3 mg). LCMS: (M+H)⁺=391.1/393.0.

### Example 11

### 1′-[(3-chloro-2-methylphenyl)sulfonyl]-3H-spiro[2-benzofuran-1,4′-piperidine]

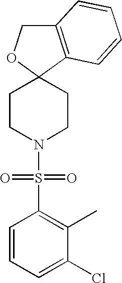

This compound was prepared using procedures analogous to those for example 10. LCMS: (M+H)⁺=378.0/380.1.

### Example 12

### 1′-[(3-chloro-2-methylphenyl)sulfonyl]spiro[chromene-2,4′-piperidine]

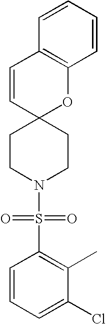

This compound was prepared using procedures analogous to those for example 10. LCMS: (M+H)⁺=390.1/392.1.

### Example 13

### 8-[(3-chloro-2-methylphenyl)sulfonyl]-2,8-diazaspiro[4.5]decan-3-one

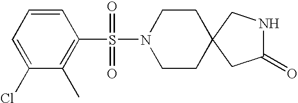

This compound was prepared using procedures analogous to those for example 10. LCMS: (M+H)⁺=343.1/345.1.

### Exampl 14

### 3-(4-Chlorophenoxy)-N-cyclohexylcyclohexanecarboxamide

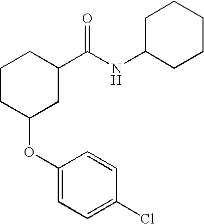

### Step 1. N-cyclohexyl-3-oxocyclohexanecarboxamide

To a mixture of 3-oxocyclohexanecarboxylic acid (1.00 g, 0.00703 mol) and benzotriazol-1-yloxytris(dimethylamino)phosphonium hexafluorophosphate (3.27 g, 0.00739 mol) in N,N-dimethylformamide (10.0 mL) was added cyclohexanamine (0.885 mL, 0.00774 mol) at 0° C. After stirring at 0° C. for 10 min, to the reaction mixture was added N,N-diisopropylethylamine (1.84 mL, 0.0106 mol). The resultant mixture was stirred at RT for 2 h, then diluted with EtOAc, washed with aq. sodium bicarbonate, water, brine, and dried with magnesium sulfate. After evaporation to dryness, the resultant crude product was used directly in next step. LCMS (M+H)⁺=224.2.

### Step 2. N-cyclohexyl-3-hydroxycyclohexanecarboxamide

Sodium tetrahydroborate (130 mg, 0.0035 mol) was added to a solution of N-cyclohexyl-3-oxocyclohexanecarboxamide (0.78 g, 0.0035 mol) in methanol (10.0 mL) at 0° C. The reaction mixture was stirred at RT for 15 min and diluted with 1 N HCl and EtOAc. The separated aqueous layer was extracted with EtOAc. The combined organic layers were washed with brine, dried and evaporated to dryness. The residue was purified on silica gel, eluting with 0 to 100% EtOAc in hexane, to give N-cyclohexyl-3-hydroxycyclohexanecarboxamide (680 mg, 86.4%). LCMS (M+H)⁺=226.2.

### Step 3. 3-(4-chlorophenoxy)-N-cyclohexylcyclohexanecarboxamide

To a solution of cis-N-cyclohexyl-3-hydroxycyclohexanecarboxamide (20 mg, 0.00009 mol) in tetrahydrofuran (0.360 mL) was added p-chlorophenol (13.7 mg, 0.000106 mol), triphenylphosphine (27.9 mg, 0.000106 mol), followed by diisopropyl azodicarboxylate (0.0210 mL, 0.000106 mol). The mixture was heated at 70° C. overnight. After concentration to dryness, the residue was purified on prep-HPLC (pH=2 conditions), to yield 3-(4-chlorophenoxy)-N-cyclohexylcyclohexanecarboxamide (18 mg, 60.38%). LCMS (M+H)⁺=336.2.

### Example 15

### 3-(2-Chlorophenoxy)-N-cyclohexylcyclohexanecarboxamide

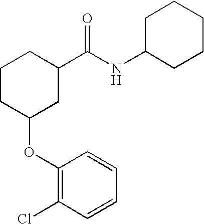

This compound was prepared using procedures analogous to those for example 14. LCMS (M+H)⁺=336.2.

### Example 16

### N-Cyclohexyl-3-(3-fluorophenoxy)cyclohexanecarboxamide

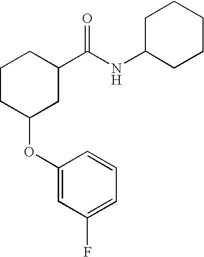

This compound was prepared using procedures analogous to those for example 14. LCMS (M+H)+=320.2.

### Example 17

### N-Cyclohexyl-3-(pyridin-2-yloxy)cyclohexanecarboxamide

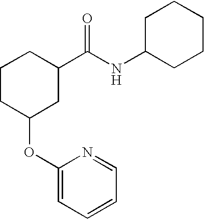

This compound was prepared using procedures analogous to those for example 14. LCMS (M+H)+=303.2.

### Example 18

### 3-[4-Chloro-3-(trifluoromethyl)phenoxy]-N-cyclohexylcyclohexanecarboxamide

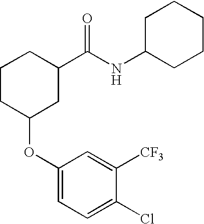

This compound was prepared using procedures analogous to those for example 14. LCMS (M+H)+=404.1.

### Example 19

### N-Cyclohexyl-3-[(4-fluorophenyl)thio]cyclohexanecarboxamide

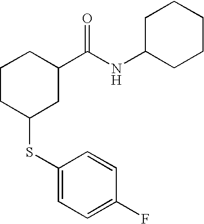

### Step 1. 3-[(cyclohexylamino)carbonyl]cyclohexyl methanesulfonate

To a mixture of N-cyclohexyl-3-hydroxycyclohexanecarboxamide (0.410 g, 0.00182 mol) and triethylamine (0.380 mL, 0.00273 mol) in methylene chloride (10.00 mL) was added methanesulfonyl chloride (0.176 mL, 0.00227 mol) at 0° C. The reaction mixture was stirred at RT for 1 h, and then washed with aq. sodium bicarbonate, dried, and evaporated to dryness. The residue was purified by silica gel chromatography to provide the desired mesylate as a white solid (492 mg, 89.1%). LCMS (M+H)⁺=304.2.

### Step 2. N-cyclohexyl-3-[(4-fluorophenyl)thio]cyclohexanecarboxamide

To a mixture of 3-[(cyclohexylamino)carbonyl]cyclohexyl methanesulfonate (40.0 mg, 0.000132 mol) and 4-fluorobenzenethiol (25.3 mg, 0.000198 mol) in N,N-dimethylformamide (0.50 mL) was added sodium hydride (10.5 mg, 0.000264 mol). The mixture was shaken at RT overnight. The resultant mixture was applied on prep-HPLC (pH=2 conditions) to give the desired product (21 mg, 47.4%). LCMS (M+H)⁺=336.1.

### Example 20

### N-Cyclohexyl-3-[(2,4-dichlorophenyl)thio]cyclohexanecarboxamide

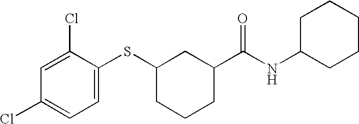

This compound was prepared using procedures analogous to those for example 19. LCMS (M+H)+=386.1.

### Example 21

### N-Cyclohexyl-3-[(2,4-dichlorophenyl)sulfonyl]cyclohexanecarboxamide

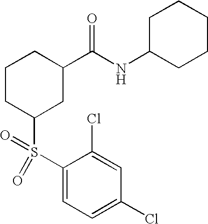

To a mixture of N-cyclohexyl-3-[(2,4-dichlorophenyl)thio]cyclohexanecarboxamide (5.0 mg, 0.000013 mol) in methanol (0.25 mL, 0.0062 mol) was added a solution of oxone® [potassium monopersulfate (11.9 mg, 0.0000388 mol)] in water (0.25 mL). The reaction mixture was stirred at RT for 2 h, and then applied on prep-HPLC (pH=2 conditions) to yield the corresponding sulfone compound (4 mg, 73.8%). LCMS (M+H)+=418.1.

### Example 22

### 3-(2-Chlorophenoxy)-N-cyclohexylcyclohexanecarboxamide

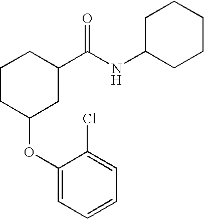

### Step 1. N-cyclohexyl-3-hydroxycyclohexanecarboxamide

A solution of potassium tri-sec-butyl(hydrido)borate(1-) in tetrahydrofuran (1.00 M, 7.34 mL) (K-Selectride) was added dropwise to a solution of N-cyclohexyl-3-oxocyclohexanecarboxamide (0.78 g, 0.0035 mol) in tetrahydrofuran (40.0 mL) at −20° C. and the mixture was stirred at the same temperature for 30 min. The reaction mixture was diluted with saturated aq. ammonium chloride and EtOAc. The separated aqueous layer was repeatedly extracted with EtOAc. The combined organic layers were washed with brine, dried and evaporated to dryness. The residue was purified by flash chromatography, eluting with 0 to 100% EtOAc in hexane, to provide the corresponding alcohol (0.67 g, 86%). LCMS (M+H)+=226.2.

### Step 2. 3-(2-chlorophenoxy)-N-cyclohexylcyclohexanecarboxamide

To a solution of N-cyclohexyl-3-hydroxycyclohexanecarboxamide (20 mg, 0.00009 mol) in tetrahydrofuran (0.360 mL) were added 2-chlorophenol (13.7 mg, 0.000106 mol) and triphenylphosphine (27.9 mg, 0.000106 mol), followed by diisopropyl azodicarboxylate (0.0210 mL, 0.000106 mol). The mixture was heated at 70° C. overnight. After concentration to dryness, the residue was purified on RP-HPLC (pH=2 conditions), to yield the desired product (8 mg, 26.8%). LCMS (M+H)⁺=336.1.

### Example 23

### N-Cyclohexyl-3-(pyridin-2-yloxy)cyclohexanecarboxamide

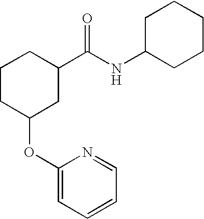

This compound was prepared using procedures analogous to those for example 22. LCMS (ESI) (M+H)⁺=303.2.

### Example 24

### (1R)-1′-[4-(4-chlorophenyl)cyclohexyl]carbonyl-3H-spiro[2-benzofuran-1,3′-pyrrolidin]-3-one

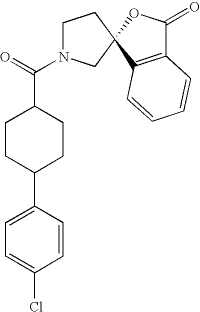

4-Methylmorpholine (28 μL, 0.25 mol) was added to a mixture of 4-(4-chlorophenyl)cyclohexanecarboxylic acid (20.0 mg, 0.0838 mmol), [(1R,4S)-7,7-dimethyl-2-oxobicyclo[2.2.1]hept-1-yl]methanesulfonic acid—(1R)-3H-spiro[2-benzofuran-1,3′-pyrrolidin]-3-one (1:1) (37.1 mg, 0.0880 mmol) and benzotriazol-1-yloxytris(dimethylamino)phosphonium hexafluorophosphate (41 mg, 0.092 mmol) in N,N-dimethylformamide (0.5 mL). The mixture was stirred at RT for 2 h, and then was diluted with methanol (1.3 mL) and adjusted with TFA to PH=2. The resulting solution was purified by prep-HPLC (pH=2 conditions) to give a TFA salt of (1R)-1′-[4-(4-chlorophenyl)cyclohexyl]carbonyl-3H-spiro[2-benzofuran-1,3′-pyrrolidin]-3-one (20.4 mg, 60%). LCMS: (M+H)⁺=410.1/412.1.

### Example 25

### N-cyclohexyl-3-(4-methoxyphenyl)cyclohex-2-ene-1-carboxamide

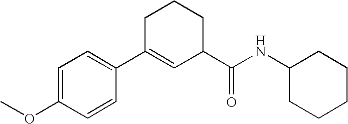

### Step 1. 3-hydroxy-3-(4-methoxyphenyl)cyclohexanecarboxylic acid

To a cooled (−20° C.) solution of 3-oxocyclohexanecarboxylic acid (0.2 g, 0.001 mol) in tetrahydrofuran (1 mL) was added a solution of p-anisyl magnesium bromide in tetrahydrofuran (0.5 M, 6 mL) with stirring. The mixture was gradually warmed up to RT over 1 h. The mixture was quenched with water, and extracted with ethyl acetate. The organic phase was washed with water and brine successively, dried over Na₂SO₄, and filtered. The filtrate was concentrated to give the crude product (quantitative yield) which was directly used in next step reaction without further purification. LCMS: (M+H)⁺=273.0.

### Step 2. N-cyclohexyl-3-(4-methoxyphenyl)cyclohex-2-ene-1-carboxamide

A mixture of 3-hydroxy-3-(4-methoxyphenyl)cyclohexanecarboxylic acid (90 mg, 0.0003 mol), cyclohexanamine (39 μL, 0.34 mmol), benzotriazol-1-yloxytris(dimethylamino)phosphonium hexafluorophosphate (1.60E2 mg, 0.362 mmol), and N,N-diisopropylethylamine (180 μL) in methylene chloride (0.3 mL) was stirred at RT for overnight. The solvent was evaporated. The residue was diluted with methanol and adjusted with TFA to pH=2. The resulting solution was purified by prep.-HPLC (pH=2 conditions) to give N-cyclohexyl-3-(4-methoxyphenyl)cyclohex-2-ene-1-carboxamide. LCMS: (M+H)⁺=314.2.

### Example 26

### N-1-adamantyl-3-oxo-1′H,3H-spiro[2-benzofuran-1,4′-piperidine]-1′-carboxamide

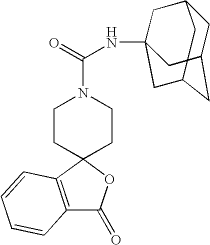

1-Isocyanatoadamantane (21.2 mg, 0.000120 mol) was added to a mixture of 3H-spiro[2-benzofuran-1,4′-piperidin]-3-one hydrochloride (34 mg, 0.14 mmol) and N,N-diisopropylethylamine (52 uL, 0.30 mmol) in acetonitrile (1.0 mL). The mixture was heated at 50° C. for overnight, and was purified by prep.-HPLC (pH=2 conditions) to give N-1-adamantyl-3-oxo-1′H,3H-spiro[2-benzofuran-1,4′-piperidine]-1′-carboxamide. LCMS: (M+H)⁺=381.2.

### Example 27

### N-1-adamantyl-1′H,3H-spiro[2-benzofuran-1,4′-piperidine]-1′-carboxamide

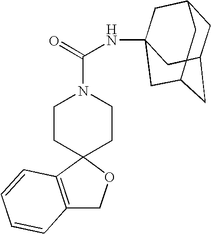

This compound was prepared using procedures analogous to those for example 26. LCMS: (M+H)⁺=367.3.

### Example 28

### N-1-adamantyl-2-oxo-1,2-dihydro-1′H-spiro[indole-3,4′-piperidine]-1′-carboxamide

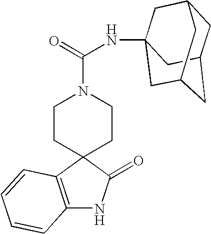

This compound was prepared using procedures analogous to those for example 26. LCMS: (M+H)⁺=380.3.

### Example 29

### N-1-adamantyl-4-oxo-1-phenyl-1,3,8-triazaspiro[4.5]decane-8-carboxamide

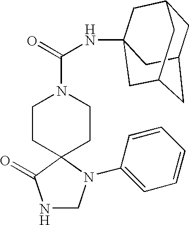

This compound was prepared using procedures analogous to those for example 26. LCMS: (M+H)⁺=409.3.

### Example 30

### 3-(4-Ethoxybenzyl)-N-(trans-4-hydroxycyclohexyl)cyclohexanecarboxamide

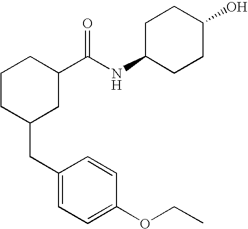

### Step 1. benzyl 3-oxocyclohexanecarboxylate

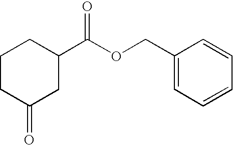

To a solution of 3-oxocyclohexanecarboxylic acid (0.90 g, 0.0063 mol) in acetonitrile (12 mL) was added benzyl bromide (0.83 mL, 0.0070 mol) at room temperature, followed by 1,8-diazabicyclo[5.4.0]undec-7-ene (0.994 mL, 0.00665 mol). The mixture was stirred at room temperature for 5 h. After removal of solvent, the residue was diluted with ethyl acetate. The organic phase was washed with aqueous NaHCO₃ solution (7.5%), 1N HCl solution, water, and brine, and dried over MgSO₄. After filtration, the filtrate was concentrated. The residue was purified by flash column to afford benzyl 3-oxocyclohexanecarboxylate. LCMS: (M+H)⁺=233.1.

### Step 2. benzyl 3-(4-ethoxybenzylidene)cyclohexanecarboxylate

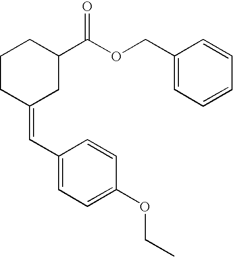

To a solution of (4-ethoxybenzyl)(triphenyl)phosphonium bromide (0.308 g, 0.000646 mol) in toluene (2.0 mL) and tetrahydrofuran (1.0 mL) was added a solution of sodium bis(trimethylsilyl)amide in tetrahydrofuran (1.00 M, 0.603 mL) at 0° C., then the mixture was stirred at 0° C. for 20 min. The reaction solution was cooled to −78° C., and a solution of benzyl 3-oxocyclohexanecarboxylate (100 mg, 0.0004 mol) in THF (1 mL) was added. The reaction mixture was warmed to RT and stirred for 3 h, and then ethyl acetate (50 mL) was added. The mixture was washed with sat. NH₄Cl solution and brine successively, and dried over MgSO₄. After filtration, the filtrate was concentrated to dryness. The residue was purified with flash column to afford benzyl 3-(4-ethoxybenzylidene)cyclohexanecarboxylate. LCMS: (M+H)⁺=351.1

### Step 3. 3-(4-ethoxybenzyl)-N-(trans-4-hydroxycyclohexyl)cyclohexanecarboxamide

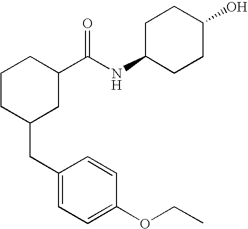

A mixture of benzyl 3-(4-ethoxybenzylidene)cyclohexanecarboxylate (50 mg, 0.0001 mol) in methanol (2 mL) and Pd/C (10%, 10 mg) was stirred under H₂ at room temperature for 2 h. The mixture was filtered. The filtrate was concentrated. The residue was dissolved in N,N-dimethylformamide (1 mL). To the solution were added trans-4-aminocyclohexanol hydrochloride (20.8 mg, 0.000137 mol), benzotriazol-1-yloxytris(dimethylamino)phosphonium hexafluorophosphate (55.6 mg, 0.000126 mol) and N,N-diisopropylethylamine (0.0498 mL, 0.000286 mol). The mixture was stirred at room temperature for 3 h, and diluted with methanol (0.8 mL). The resulting solution was purified by prep-HPLC (pH=2 conditions) to afford 3-(4-ethoxybenzyl)-N-(trans-4-hydroxycyclohexyl)cyclohexanecarboxamide. LCMS: (M+H)⁺=360.2.

### Example 31

### 4-(4-Bromo-2-methylphenyl)-4-hydroxy-N-(cis-4-hydroxycyclohexyl)cyclohexanecarboxamide

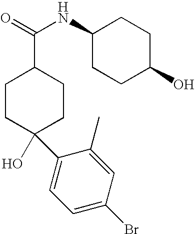

### Step 1. Ethyl 4-(4-bromo-2-methylphenyl)-4-hydroxycyclohexanecarboxylate

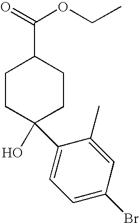

A solution of isopropylmagnesium bromide in tetrahydrofuran (1.0 M, 7.6 mL, 7.6 mmol) was added into a solution of 4-bromo-1-iodo-2-methylbenzene (2.1 g, 7.0 mmol) in tetrahydrofuran (10.0 mL) under an atmosphere of nitrogen at −40° C. The mixture was warmed to room temperature and stirred for 30 min, then was cooled to −78° C. To the mixture was added ethyl 4-oxocyclohexanecarboxylate (1.0 g, 5.9 mol) in tetrahydrofuran (2.0 mL). The reaction mixture was allowed to warm slowly to room temperature, and stirred at room temperature for an additional 30 min. The mixture was quenched with sat. aqueous NH₄Cl solution, and extracted with ethyl acetate. The organic layer was washed with brine, dried over Na₂SO₄, and filtered. The filtrate was concentrated. The residue was purified by flash chromatography (ethyl acetate in hexane: 0-50%) to give ethyl 4-(4-bromo-2-methylphenyl)-4-hydroxycyclohexanecarboxylate (0.46 g, 23%).

### Step 2. 4-(4-Bromo-2-methylphenyl)-4-hydroxycyclohexanecarboxylic acid

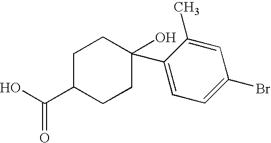

A mixture of ethyl 4-(4-bromo-2-methylphenyl)-4-hydroxycyclohexanecarboxylate (0.11 g, 0.32 mmol) and lithium hydroxide monohydrate (0.054 g, 1.3 mmol) in methanol (1.0 mL) and water (1.0 mL) was stirred at room temperature for overnight. The mixture was acidified with HCI (4.0 M) (to pH˜5). The solvents were evaporated under reduced pressure to afford the desire product which was directly used in next step reaction without further purification. LCMS: (M+H−H₂O)⁺=295.0/297.0.

### Step 3. 4-(4-Bromo-2-methylphenyl)-4-hydroxy-N-(cis-4-hydroxycyclohexyl)cyclohexanecarboxamide

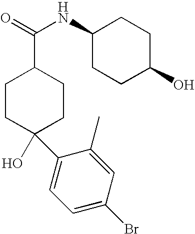

4-Methylmorpholine (0.10 mL, 0.96 mmol) was added to a mixture of 4-(4-bromo-2-methylphenyl)-4-hydroxycyclohexanecarboxylic acid (0.32 mmol), cis-4-aminocyclohexanol hydrochloride (0.0508 g, 0.335 mmol) and benzotriazol-1-yloxytris(dimethylamino)phosphonium hexafluorophosphate (0.155 g, 0.351 mmol) in N,N-dimethylformamide (3.0 mL). The mixture was stirred at room temperature for overnight. The solvents were evaporated under reduced pressure. The residue was flash chromatographed on a silica gel column (ethyl acetate in hexanes: 0-90%) to give 4-(4-bromo-2-methylphenyl)-4-hydroxy-N-(cis-4-hydroxycyclohexyl)cyclohexanecarboxamide (110 mg, 84%). LCMS: (M+H)⁺=410.1/412.1.

### Example 32

### 4-(4-Bromo-2-methylphenyl)-N-(cis-4-hydroxycyclohexyl)cyclohex-3-ene-1-carboxamide

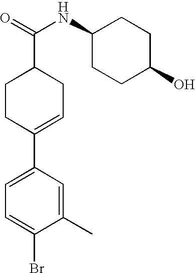

A mixture of trifluoroacetic acid (1.0 mL, 0.013 mol) and 4-(4-bromo-2-methylphenyl)-4-hydroxy-N-(cis-4-hydroxycyclohexyl)cyclohexanecarboxamide (35.0 mg, 0.0853 mmol) in methylene chloride (1.0 mL) was stirred at room temperature for overnight. The solvents were evaporated under reduced pressure. The residue was dissolved in methanol and the solution was treated with NaOH (aq. 1.0 M) for 1 h. The mixture extracted with dichloromethylene. The organic phase was washed with brine, dried over Na₂SO₄, filtered and concentrated. The residue was purified by prep-HPLC (pH=2 conditions) to afford 4-(4-bromo-2-methylphenyl)-N-(cis-4-hydroxycyclohexyl)cyclohex-3-ene-1-carboxamide (20 mg, 60%). LCMS: (M+H)⁺=392.1/394.1

### Example 33

### 5-[4-(1-Hydroxy-4-{[(cis-4-hydroxycyclohexyl)amino]carbonyl}cyclohexyl)-3-methylphenyl]-N-methylpyridine-2-carboxamide

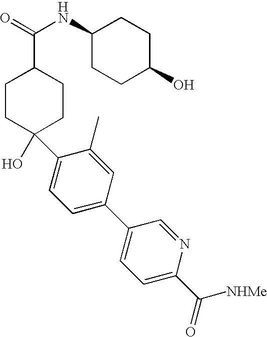

A mixture of N-methyl-5-(4,4,5,5-tetramethyl-1,3,2-dioxaborolan-2-yl)pyridine-2-carboxamide (14 mg, 0.055 mmol), tetrakis(triphenylphosphine)palladium(0) (1.6 mg, 0.0014 mmol), K₃PO₄ (23 mg, 0.11 mmol), and 4-(4-bromo-2-methylphenyl)-4-hydroxy-N-(cis-4-hydroxycyclohexyl)-cyclohexane-carboxamide (15.0 mg, 0.0366 mmol) in 1,4-dioxane (0.3 mL) and water (0.3 mL) was stirred at 120° C. for 2 h. After cooling, the mixture was filtered. The filtrate was diluted with methanol (1.3 mL) and purified by prep-HPLC (pH=2 conditions) to afford 5-[4-(1-hydroxy-4-{[(cis-4-hydroxycyclohexyl)-amino]-carbonyl}cyclohexyl)-3-methylphenyl]-N-methylpyridine-2-carboxamid (15 mg). LCMS: (M+H)⁺=466.2.

### Example 34

### 4′-(1-Hydroxy-4-{[(cis-4-hydroxycyclohexyl)amino]carbonyl}cyclohexyl)-N,3′-dimethylbiphenyl-4-carboxamide

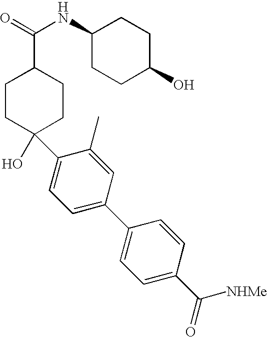

This compound was prepared using procedures analogous to those for example 33. LCMS: (M+H)⁺=465.2.

### Example 35

### 1′-(3-Chloropyridin-2-yl)spiro[indole-3,4′-piperidin]-2(1H)-one

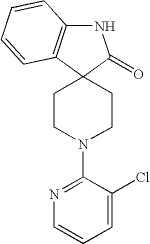

A mixture of spiro[indole-3,4′-piperidin]-2(1H)-one hydrochloride (11.5 mg), 2,3-dichloropyridine (7.6 mg, 1.0 eq.) and diisiopropylethylamine (25.0 μL) in N-methylpyrrolidinone (0.5 mL) was heated at 180° C. for 30 min. After cooling, the mixture was diluted with methanol (1.3 mL) and adjusted with TFA to be acidic (pH˜2.0). The resulting solution was purified by prep-HPLC (pH=2 conditions) to give a TFA salt of 1′-(3-chloropyridin-2-yl)spiro[indole-3,4′-piperidin]-2(1H)-one (14 mg, 92%). LCMS: (M+H)⁺=314.1/316.1.

### Example 36

### 4′-(4-[(cis-4-hydroxycyclohexyl)amino]carbonylcyclohex-1-en-1-yl)-N,3′-dimethylbiphenyl-4-carboxamide

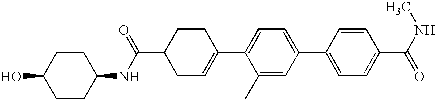

Method A: A mixture of 4-[(methylamino)carbonyl]phenylboronic acid (9.8 mg, 0.000055 mol), Tetrakis(triphenylphosphine)palladium(0) (1.6 mg, 0.0000014 mol), K₃PO₄ (23 mg, 0.00011 mol), and 4-(4-bromo-2-methylphenyl)-N-(cis-4-hydroxycyclohexyl)cyclohex-3-ene-1-carboxamide (14.3 mg, 0.0000366 mol) in 1,4-Dioxane (0.3 mL) and water (0.3 mL) was stirred at 120° C. for 2 h. After cooling, the mixture was filtered, and the filtrate was diluted with methanol (1.3 mL) and was purified by prep-LCMS (pH=2 conditions) to give 4′-(4-[(cis-4-hydroxycyclohexyl)amino]carbonylcyclohex-1-en-1-yl)-N,3′-dimethylbiphenyl-4-carboxamide (10 mg, 61.2%).

Method B: 0.5 mL of con. HCl was added into a solution of 4′-(1-hydroxy-4-[(cis-4-hydroxycyclohexyl)amino]carbonylcyclohexyl)-N,3′-dimethylbiphenyl-4-carboxamide (0.010 g, 0.000021 mol) in dioxane (0.5 mL). The mixture was stirred at room temperature for 2 h. The solvents were evaporated under reduced pressure. The residue was diluted with methanol (1.8 mL), and was purified by prep-HPLC (pH=2 conditions) to afford 7.5 mg of 4′-(4-[(cis-4-hydroxycyclohexyl)amino]carbonylcyclohex-1-en-1-yl)-N,3′-dimethylbiphenyl-4-carboxamide. LCMS: (M+H)⁺=447.1.

### Example 37

### 4′-(4-{[(cis-4-hydroxycyclohexyl)amino]carbonyl}cyclohexyl)-N,3′-dimethylbiphenyl-4-carboxamide

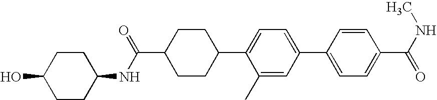

A mixture of 4′-(4-[(cis-4-hydroxycyclohexyl)amino]carbonylcyclohex-1-en-1-yl)-N,3′-dimethylbiphenyl-4-carboxamide (5.0 mg) and palladium on carbon (10%, 2.0 mg) in methanol (2 ml) was stirred at room temperature for 2 h. Then the mixture was filtered. The filtrated was concentrated to give 5.0 mg of 4′-(4-{[(cis-4-hydroxycyclohexyl)amino]carbonyl}cyclohexyl)-N,3′-dimethylbiphenyl-4-carboxamide. LCMS: (M+H)⁺=449.2.

### Example 38

### 5-[4-(4-[(cis-4-hydroxycyclohexyl)amino]carbonylcyclohex-1-en-1-yl)-3-methylphenyl]-N-methylpyridine-2-carboxamide

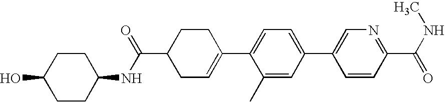

This compound was prepared using procedures analogous to those for example 36. LCMS: (M+H)⁺=448.2.

### Example 39

### N-(cis-4-hydroxycyclohexyl)-4-(2-methylphenyl)cyclohex-3-ene-1-carboxamide

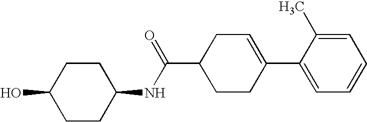

A mixture of 4-(4-bromo-2-methylphenyl)-N-(cis-4-hydroxycyclohexyl)cyclohex-3-ene-1-carboxamide (0.015 g, 0.000038 mol) and platinum on carbon (5%, 5 mg) in methanol (2.0 m) was stirred under an atmosphere of hydrogen for 1 h. Then the mixture was filtered and filtrate was concentrated. The residue was diluted with methanol (1.8 mL) and purified by prep-HPLC (pH=2 conditions) to give 11.5 mg of N-(cis-4-hydroxycyclohexyl)-4-(2-methylphenyl)cyclohex-3-ene-1-carboxamide. LCMS: (M+H)⁺=314.2.

### Example 40

### N-(cis-4-hydroxycyclohexyl)-4-(2-methylphenyl)cyclohexanecarboxamide

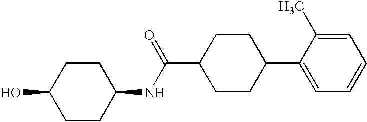

A mixture of N-(cis-4-hydroxycyclohexyl)-4-(2-methylphenyl)cyclohex-3-ene-1-carboxamide (9.0 mg) and palladium on carbon (10%, 5 mg) in methanol (2.0 mL) was stirred under an atmosphere of hydrogen for 1 h. The mixture was filtered. The filtrate was concentrated. The residue was diluted with methanol (1.8 mL) and purified by prep-HPLC (pH=2 conditions) to give 8.2 mg of N-(cis-4-hydroxycyclohexyl)-4-(2-methylphenyl)cyclohexanecarboxamide. LCMS: (M+H)⁺=316.2.

### Example 41

### 1′-(piperidin-1-ylcarbonyl)-3H-spiro[2-benzofuran-1,4′-piperidin]-3-one

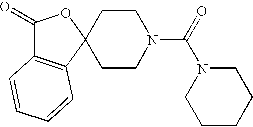

### 1-Piperidinecarbonyl chloride (15.0 μL, 0.000120 mol) was added to a mixture of 3H-spiro[2-benzofuran-1,4′-piperidin]-3-one hydrochloride (34 mg, 0.00014 mol) and N,N-diisopropylethylamine (52 μL, 0.00030 mol) in acetonitrile (1.0 mL). The reaction mixture was stirred at room temperature for overnight. After cooling, the mixture was diluted with methanol (0.8 mL), and was purified by prep-LCMS (pH=2 conditions) to give 1′-(piperidin-1-ylcarbonyl)-3H-spiro[2-benzofaran-1,4′-piperidin]-3-one (25 mg, 66.5%). LCMS: (M+H)⁺=315.2.

### Example 42

### 1′-(piperidin-1-ylcarbonyl)-3H-spiro[2-benzofuran-1,4′-piperidine]

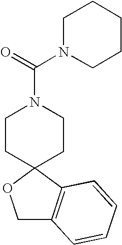

This compound was prepared using procedures analogous to those for example 41. LCMS: (M+H)⁺=301.2.

### Example 43

### 1′-(piperidin-1-ylcarbonyl)spiro[indole-3,4′-piperidin]-2(1H)-one

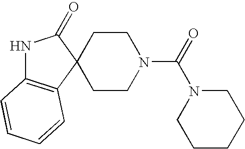

This compound was prepared using procedures analogous to those for example 41. LCMS: (M+H)⁺=314.2.

### Example 44

### 8-(piperidin-1-ylcarbonyl)-1-phenyl-1,3,8-triazaspiro[4.5]decan-4-one

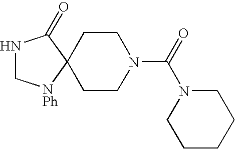

This compound was prepared using procedures analogous to those for example 41. LCMS: (M+H)⁺=343.2.

### Example 45

### 7-(5-bromo-3-chloropyridin-2-yl)-2-(cyclohexylcarbonyl)-2,7-diazaspiro[4.5]decane

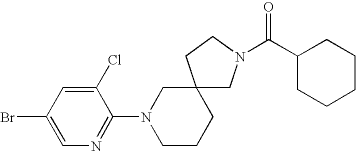

### Step 1: 7-(5-bromo-3-chloropyridin-2-yl)-2,7-diazaspiro[4.5]decane hydrochloride

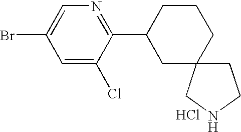

A mixture of tert-butyl 2,7-diazaspiro[4.5]decane-2-carboxylate hydrochloride (111 mg, 0.000400 mol), 5-bromo-2,3-dichloropyridine (95.3 mg, 0.000420 mol) and potassium carbonate (170 mg, 0.0012 mol) in N,N-dimethylformamide (3.3 mL) was heated at 180° C. for 30 min. After cooling, the mixture was filtered. The filtrate was concentrated under reduced pressure. The residue was treated with 4.0 M of hydrogen chloride in 1,4-dioxane (1.00 mL) at room temperature for 3 h. The solvent was evaporated. The residue was co-evaporated with toluene (2×). The residue was 7-(5-bromo-3-chloropyridin-2-yl)-2,7-diazaspiro[4.5]decane hydrochloride which was directly used in next step reaction without further purification.

### Step 2. 7-(5-bromo-3-chloropyridin-2-yl)-2-(cyclohexylcarbonyl)-2,7-diazaspiro[4.5]decane

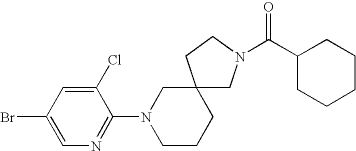

N,N-Diisopropylethylamine (40.0 μL, 0.000230 mol) was added to 7-(5-bromo-3-chloropyridin-2-yl)-2,7-diazaspiro[4.5]decane hydrochloride (18.4 mg, 0.0000500 mol) in acetonitrile (0.50 mL), followed by cyclohexanecarbonyl chloride (8.15 μL, 0.0000600 mol). The mixture was stirred at room temperature for overnight, and then was diluted with methanol (1.3 mL). The resulting solution was purified by prep-HPLC (pH=2 conditions) to afford 14.5 mg of a TFA salt of 7-(5-bromo-3-chloropyridin-2-yl)-2-(cyclohexylcarbonyl)-2,7-diazaspiro[4.5]decane. LCMS: (M+H)⁺=440.1/442.1.

### Example 46

### 7-(5-bromo-3-chloropyridin-2-yl)-N-cyclohexyl-2,7-diazaspiro[4.5]decane-2-carboxamide

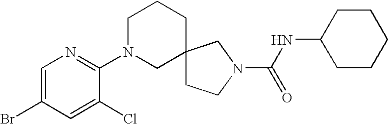

N,N-Diisopropylethylamine (40.0 μL, 0.000230 mol) was added to 7-(5-bromo-3-chloropyridin-2-yl)-2,7-diazaspiro[4.5]decane hydrochloride (18.4 mg, 0.0000500 mol) in acetonitrile (0.50 mL), followed by cyclohexylisocyanate (7.51 mg, 0.0000600 mol). The mixture was stirred at room temperature for overnight, and then was diluted with methanol (1.3 mL). The resulting solution was purified by prep-HPLC (pH=2 conditions) to afford 13.3 mg of a TFA salt of 7-(5-bromo-3-chloropyridin-2-yl)-N-cyclohexyl-2,7-diazaspiro[4.5]decane-2-carboxamide. LCMS: (M+H)⁺=455.1/457.1.

### Example 47

### 7-(5-bromo-3-chloropyridin-2-yl)-2-(piperidin-1-ylcarbonyl)-2,7-diazaspiro[4.5]decane

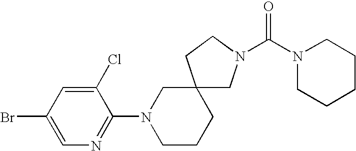

This compound was prepared using procedures analogous to those for example 45. LCMS: (M+H)⁺=441.0/443.0.

### Example 48

### 7-(5-bromo-3-chloropyridin-2-yl)-2-(morpholin-4-ylcarbonyl)-2,7-diazaspiro[4.5]decane

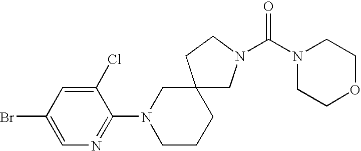

This compound was prepared using procedures analogous to those for example 45. LCMS: (M+H)⁺=443.0/445.0.

### Example 49

### 7-(5-bromo-3-chloropyridin-2-yl)-2-(3-methoxybenzoyl)-2,7-diazaspiro[4.5]decane

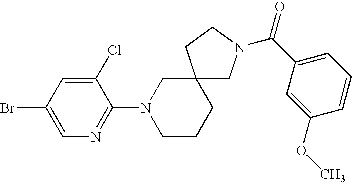

This compound was prepared using procedures analogous to those for example 45. LCMS: (M+H)⁺=464.0/466.0.

### Example 50

### 7-(5-bromo-3-chloropyridin-2-yl)-2-(2-chlorobenzoyl)-2,7-diazaspiro[4.5]decane

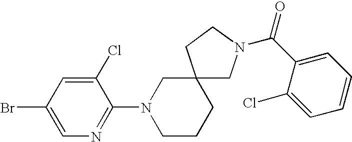

This compound was prepared using procedures analogous to those for example 45. LCMS: (M+H)⁺=468.0/470.0.

### Example 51

### 2-(1-adamantylcarbonyl)-7-(5-bromo-3-chloropyridin-2-yl)-2,7-diazaspiro[4.5]decane

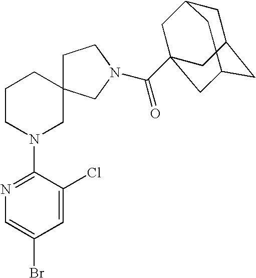

This compound was prepared using procedures analogous to those for example 45. LCMS: (M+H)⁺=492.1/494.1

### Example 52

### N-1-adamantyl-7-(5-bromo-3-chloropyridin-2-yl)-2,7-diazaspiro[4.5]decane-2-carboxamide

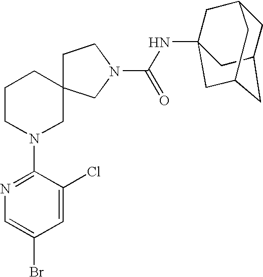

This compound was prepared using procedures analogous to those for example 46. LCMS: (M+H)⁺=507.1/509.1.

### Example 53

### 8-(4-Chlorophenyl)-2-(trans-4-hydroxycyclohexyl)-2-azaspiro[4.5]decan-1-one

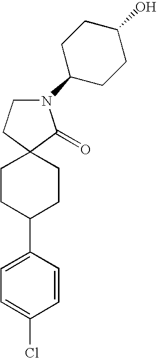

### Step 1: Methyl 1-allyl-4-(4-chlorophenyl)cyclohexanecarboxylate

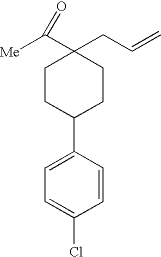

A solution of 1.0 M of lithium hexamethyldisilazide in tetrahydrofuran (5.1 mL) was added to a solution of methyl 4-(4-chlorophenyl)cyclohexanecarboxylate (1.0 g, 0.0040 mol) in tetrahydrofuran (8 mL) at −78° C. The mixture was stirred at −78° C. for 1 hour. To the mixture was added allyl bromide (510 uL, 0.0059 mol). The mixture was stirred at −78° C. for 30 min, and was allowed to warm to room temperature overnight. The mixture was diluted with ethyl acetate, washed with NaHCO₃ (7.5%), 1N HCl solution, water, and brine, dried over MgSO₄. filtered, and concentrated. The residue was flash chromatographed on a silica gel column to afford the desired product (0.622 g, 53.7%). ¹H NMR (CDCl₃): 6 (ppm): 1.04˜1.36 (m, 2H), 1.42˜1.52 (m, 2H), 1.76˜1.84 (m, 2H), 2.24˜2.28 (d, 2H), 2.32˜2.38 (m, 2H), 2.40˜2.52 (m, 1H), 3.76 (s, 3H), 5.04˜5.12 (m, 2H), 5.68˜5.80 (m, 1H), 7.04 (d, 2H), 7.22 (d, 2H).

### Step 2: Methyl 4-(4-chlorophenyl)-1-(2-oxoethyl)cyclohexanecarboxylate

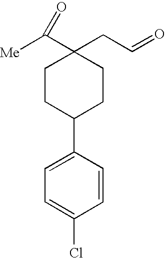

A solution of methyl 1-allyl-4-(4-chlorophenyl)cyclohexanecarboxylate (0.62 g, 0.0021 mol) in methylene chloride (10 mL) at −78° C. was passed through a ozone gas which was generated by ozonous system. After the solution turned into blue, it was treated with oxygen gas then dimethyl sulfide (0.31 mL, 0.0042 mol). The mixture was stirred at room temperature overnight. After removal of solvent the crude material was purified with flash column on a silica gel column to afford the desired product (0.387 g, 62%). ¹H NMR (CDCl₃): δ (ppm): 1.34˜1.50 (m, 2H), 1.54˜1.72 (m, 2H), 1.72˜1.82 (m, 2H), 2.32˜2.56 (m, 3H), 2.58 (d, 2H), 3.76 (s, 3H), 7.04 (d, 2H), 7.22 (d, 2H), 9.70 (s, 1H).

### Step3: 8-(4-chlorophenyl)-2-(trans-4-hydroxycyclohexyl)-2-azaspiro[4.5]decan-1-one

A mixture of methyl 4-(4-chlorophenyl)-1-(2-oxoethyl)cyclohexanecarboxylate (0.180 g, 0.000611 mol), trans-4-aminocyclohexanol hydrochloride (0.10 g, 0.00067 mol), triethylamine (140 μL, 0.00098 mol) in 1,2-dichloroethane (2 mL) was stirred at RT for 30 min. To the mixture was added sodium triacetoxyborohydride (0.32 g, 0.0015 mol). The mixture was stirred at r.t. for 2 h, then it was heated at 120° C. overnight. After cooling, the mixture was diluted with methylene chloride, washed with water and brine, and concentrated. The residue was diluted with methanol, and purified by prep.-HPLC (pH=10 conditions) to give two isomers: fraction I, 1.3 mg; and fraction II, 12.9 mg. LCMS confirmed both products. Retention times for analytical LC/MS were as follows: Isomer-I: Rt=2.189 min; Isomer-II: Rt=2.329 min. LCMS: (M+H)⁺=362.1.

### Example 54

### 8-(4-Bromophenoxy)-2-(trans-4-hydroxycyclohexyl)-2-azaspiro[4.5]decan-1-one

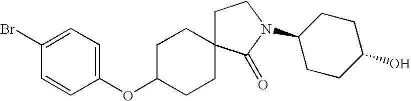

### Step 1: Ethyl 4-(4-bromophenoxy)cyclohexanecarboxylate

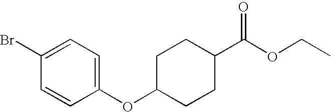

A solution of diisopropyl azodicarboxylate (3.4 mL, 0.017 mol) in THF (5 mL) was added dropwise to a solution of ethyl 4-hydroxycyclohexanecarboxylate (1.50 g, 0.00871 mol), 4-bromophenol (3.0 g, 0.017 mol), and triphenylphosphine (4.6 g, 0.017 mol) in THF (10 mL) at −20° C. After addition, the mixture was stirred at RT for 2 days. The mixture was concentrated. The crude residue was purified by flash column chromatography to yield the desired product (1.20 g, 42.11%). LCMS: (M+H)⁺=327.0; (M-4-BrPh)+=155.2.

### Step2: Ethyl1-allyl-4-(4-bromophenoxy)cyclohexanecarboxylate

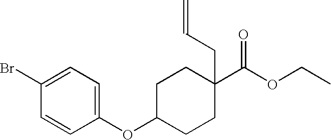

1.0 M of isopropylmagnesium bromide in tetrahydrofuran (3.8 mL) was added slowly to a solution of ethyl 4-(4-bromophenoxy)cyclohexanecarboxylate (0.95 g, 0.0029 mol) in tetrahydrofuran (7 mL) at −78° C. The mixture was stirred for 1 h at −78° C., and 30 min at −50° C. The mixture was re-cooled to −78° C., and allyl bromide (0.53 g, 0.0044 mol) was added. The mixture was stirred at ambient temperature overnight. The mixture was quenched with NaHCO₃ solution (7.5%), extracted with ethyl acetate. The extract was washed with citric acid (10%), water, and brine, and dried over MgSO₄, filtered, and concentrated. The crude residue was purified by flash column chromatography to yield the desired product (595 mg, 55.8%). ¹H NMR (CDCl₃): δ (ppm): 1.24 (t, 3H), 1.42˜2.38 (m, 11H), 4.12 (q, 2H), 5.02 (m, 2H), 5.70 (m, 1H), 6.76 (d, 2H), 7.36 (d, 2H).

### Step3: Ethyl 4-(4-bromophenoxy)-1-(2-oxoethyl)cyclohexanecarboxylate

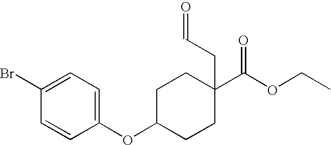

A solution of methyl 1-allyl-4-(4-bromophenoxy)cyclohexanecarboxylate (580 mg, 1.58 mmol) in methylene chloride (10 mL) at −78° C. was passed through a ozone gas which was generated by ozonous system. After the solution turned into blue, it was treated with oxygen gas then dimethyl sulfide (0.29 mL, 0.0039 mol). The mixture was stirred at room temperature overnight. After removal of solvent the crude material was purified with flash column on a silica gel column to afford the desired product (0.332 g, 56.9%). LCMS: (M+H)⁺=369.0.

### Step 4: 8-(4-Bromophenoxy)-2-(trans-4-hydroxycyclohexyl)-2-azaspiro[4.5]decan-1-one

A mixture of ethyl 4-(4-bromophenoxy)-1-(2-oxoethyl)cyclohexanecarboxylate (0.33 g, 0.00089 mol), trans-4-aminocyclohexanol hydrochloride (0.15 g, 0.00089 mol), triethylamine (190 μL, 0.0013 mol) in 1,2-dichloroethane (3 mL) was stirred at RT for 30 min. To the mixture was added sodium triacetoxyborohydride (0.47 g, 0.0022 mol). The mixture was stirred at r.t. for 2 h, then it was heated at 120° C. overnight. After cooling, the mixture was diluted with methylene chloride, washed with water and brine, and concentrated. The residue was diluted with methanol, and purified by prep.-HPLC (pH=10 conditions) to give the desired product (342 mg, 90.61%). LCMS: (M+H)⁺=422.1.

### Example 55

### 3-(cis-4-Hydroxycyclohexyl)-1-methyl-8-phenyl-1,3-diazaspiro[4.5]decane-2,4-dione

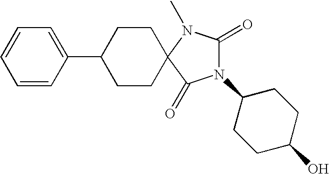

### Step 1: 1-(Methylamino)-4-phenylcyclohexanecarbonitrile

A mixture of 4-phenylcyclohexanone (2.0 g, 0.011 mol), methylammonium chloride (0.93 g, 0.014 mol), and potassium cyanide (0.90 g, 0.014 mol) in ethanol (30 mL) and water (8 mL) was stirred at RT overnight. The mixture was concentrated. The white solid formed was filtered, washed with water, and dried under high vacuum to yield the desired product (2.39 g, 97.16%). LCMS: (M+H)⁺=215.2.

### Step 2: 1-Methyl-8-phenyl-1,3-diazaspiro[4.5]decane-2,4-dione

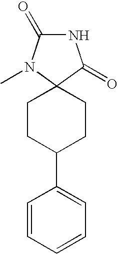

To a solution of 1-(methylamino)-4-phenylcyclohexanecarbonitrile (1.0 g, 0.0047 mol) in acetic acid (6 mL) was added dropwise a solution of potassium cyanate (0.76 g, 0.0093 mol) in water (2.0 mL). The mixture was stirred for 1 h at 50° C., and 1 h at 60° C. To the solution was added 3 mL of conc. HCl solution. The resulting solution was heated at 60° C. for 1 h. After cooling, the mixture was concentrated. The white solid was filtered, washed with a little of warer, and dried under vacuum to yield the desired product (940 mg, 78%). LCMS: (M+H)⁺=259.1.

### Step 3: 4-[tert-Butyl(dimethyl)silyl]oxycyclohexanol

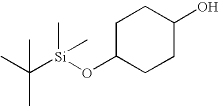

Sodium tetrahydroborate (0.41 g, 0.011 mol) was added in small portions to a solution of 4-[tert-butyl(dimethyl)silyl]oxycyclohexanone (1.0 g, 0.0044 mol) in methanol (10 mL) at −50° C. The mixture was gradually warmed to RT, and was diluted with ethyl acetate. The organic solution was washed with NaHCO₃ solution (7.5%), water, and brine, dried over MgSO₄, filtered, and concentrated to yield the desired product (0.94 g, 93%) which was directly used in next step reaction without further purification. LCMS: (M+H)⁺=231.2.

### Step 4: 3-(cis-4-Hydroxycyclohexyl)-1-methyl-8-phenyl-1,3-diazaspiro[4.5]decane -2,4-dione

Diethyl azodicarboxylate (120 μL, 0.00077 mol) was added to a mixture of 1-methyl-8-phenyl-1,3-diazaspiro[4.5]decane-2,4-dione (100.0 mg, 0.00039 mol), 4-[tert-butyl(dimethyl)silyl]oxycyclohexanol (130 mg, 0.00058 mol), and triphenylphosphine (200.0 mg, 0.00077 mol) in THF (1 mL) at RT. The mixture was stirred at RT overnight. To the above reaction mixture was added 1.69 M of fluorosilicic acid in water (0.69 mL). The mixture was stirred at RT for 3 h, and then was purified by prep.-HPLC (pH=10 conditions) to afford two isomers. Retention times for analytical LC/MS were as follows: Isomer-I: Rt=1.831 min; Isomer-II: Rt=1.877 min. LCMS: Fraction I: m/z 357.2 (M+H)+; 379.2 (M+Na)+; LCMS: Fraction II: m/z 357.2 (M+H)+; 379.2 (M+Na)+; 339.2 (M−H2O)+; 735.3 (2M+Na)+.

### Example 56

### N-[1-(4-Cyanophenyl)-4-methylpiperidin-4-yl]-2-oxa-6-azatricyclo[3.3.1.1(3,7)]decane-6-carboxamide

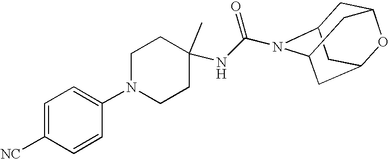

### Step1: tert-Butyl 4-methyl-4-[(2-oxa-6-azatricyclo[3.3.1.1(3,7)]dec-6-ylcarbonyl)amino]piperidine-1-carboxylate

To a stirred solution of 1-(tert-butoxycarbonyl)-4-methylpiperidine-4-carboxylic acid (0.500 g, 0.00206 mol) in tetrahydrofuran (10.00 mL) was added diphenylphosphonic azide (0.465 mL, 0.00216 mol) and triethylamine (0.859 mL, 0.00616 mol), and the mixture was refluxed for 1 h under nitrogen. The reaction mixture was then treated with 2-oxa-6-azatricyclo[3.3.1.1(3,7)]decane hydrochloride (0.379 g, 0.00216 mol) at reflux overnight. The mixture was concentrated under reduced pressure, diluted with EtOAc, and washed with aq. sodium bicarbonate. The organic layers were combined, washed with brine, dried, and evaporated to dryness. The crude urea was used directly in next step. LCMS: (M+H)+=380.2.

### Step 2:N-(4-methylpiperidin-4-yl)-2-oxa-6-azatricyclo[3.3.1.1(3,7)]decane-6-carboxamide hydrochloride

tert-Butyl 4-methyl-4-[(2-oxa-6-azatricyclo[3.3.1.1(3,7)]dec-6-ylcarbonyl)amino]piperidine-1-carboxylate (0.78 g, 0.0020 mol) was treated with 4.00 M of hydrogen chloride in 1,4-dioxane (10.0 mL) at RT for 2 h. After evaporation to dryness, the resultant HCl salt was used directly in next step. LCMS: (M+H)+=280.2.

### Step3: N-[1-(4-cyanophenyl)-4-methylpiperidin-4-yl]-2-oxa-6-azatricyclo[3.3.1.1(3,7)]decane-6-carboxamide

To a mixture of crude N-(4-methylpiperidin-4-yl)-2-oxa-6-azatricyclo[3.3.1.1(3,7)]decane-6-carboxamide hydrochloride (20.0 mg, 0.0000633 mol), (4-cyanophenyl)boronic acid (0.0279 g, 0.000190 mol), copper acetate (19.4 mg, 0.000158 mol), and molecular sieves (63.8 mg, 0.000285 mol) in methylene chloride (0.586 mL, 0.00915 mol) was added triethylamine (0.0441 mL, 0.000317 mol). The resultant mixture was stirred at rt overnight. After evaporation to dryness, the residue was diluted with AcCN and filtered through a 0.4 U memberane. The filtration was diluted with water and applied on RP-HPLC to give the product (15 mg, 62.26%). LCMS: (M+H)+=381.2.

### Example 57

### N-4-Methyl-1-[4-(trifluoromethyl)phenyl]piperidin-4-yl-2-oxa-6-azatricyclo[3.3.1.1(3,7)]decane-6-carboxamide

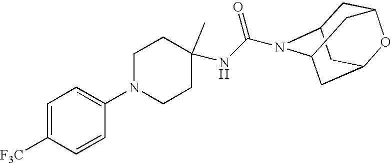

This compound was prepared by using a procedure analogous to that described for the synthesis of example 56. LCMS: (M+H)+=424.2.

### Example 58

### N-[1-(2-Fluoro-4-methylphenyl)-4-methylpiperidin-4-yl]-2-oxa-6-azatricyclo[3.3.1.1(3,7)]decane-6-carboxamide

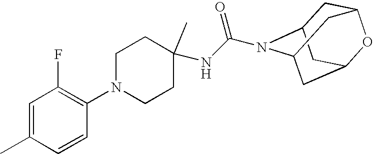

This compound was prepared by using a procedure analogous to that described for the synthesis of example 56. LCMS: (M+H)+=388.2.

### Example 59

### N-[1-(2-Chlorophenyl)-4-methylpiperidin-4-yl]-2-oxa-6-azatricyclo[3.3.1.1(3,7)]decane-6-carboxamide

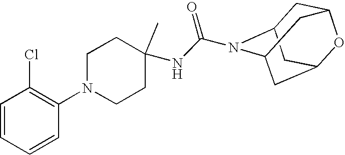

This compound was prepared by using a procedure analogous to that described for the synthesis of example 56. LCMS: (M+H)+=390.2.

### Example 60

### N-[1-(2,3-difluorophenyl)-4-methylpiperidin-4-yl]-2-oxa-6-azatricyclo[3.3.1.1(3,7)]-decane-6-carboxamide

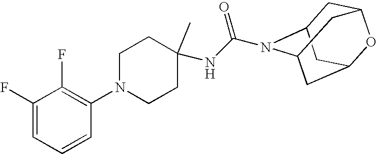

This compound was prepared by using a procedure analogous to that described for the synthesis of example 56. LCMS: (M+H)+=392.2.

### Example 61

### 1-[(3S)-1-(4-Bromo-2-fluorophenyl)piperidin-3-yl]-3-(cis-4-hydroxycyclohexyl)-tetrahydropyrimidin-2(1H)-one

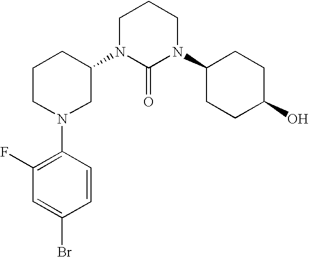

### Step 1: (3S)-1-(4-bromo-2-fluorophenyl)piperidin-3-amine dihydrochloride

tert-Butyl [(3S)-1-(4-bromo-2-fluorophenyl)piperidin-3-yl]carbamate (1.09 g, 0.00292 mol) was treated with 4.0 M of hydrogen chloride in 1,4-dioxane (15 mL). The mixture was stirred at RT for 2 h and then the solvent was evaporated to yield the desired product (1.28 g, 126.66%).

### Step 2: ethyl 3-[(3S)-1-(4-bromo-2-fluorophenyl)piperidin-3-yl]amino-3-oxopropanoate

N-(3-Dimethylaminopropyl)-N′-ethylcarbodiimide hydrochloride (426 mg, 0.00222 mol) was added to a mixture of monoethyl malonate (230 μL, 0.0019 mol), (3S)-1-(4-bromo-2-fluorophenyl)piperidin-3-amine dihydrochloride (630 mg, 0.0018 mol), 1-hydroxybenzotriazole (267 mg, 0.00198 mol), and triethylamine (1.4 mL, 0.010 mol) in methylene chloride (20 mL). The mixture was stirred at RT for 3 d, and was then concentrated. The residue was purified on a silica gel column (0-40-60% EtOAc/hex) to give the desired product (478 mg) as white solid. LCMS (M+H)+=387.

### Step 3: 3-[(3S)-1-(4-bromo-2-fluorophenyl)piperidin-3-yl]amino-3-oxopropanoic acid

1.0 M of lithium hydroxide in water (3.7 mL) was added to ethyl 3-[(3S)-1-(4-bromo-2-fluorophenyl)piperidin-3-yl]amino-3-oxopropanoate (485 mg, 0.00125 mol) in tetrahydrofuran (20 mL). The mixture was stirred at RT overnight. The mixture was washed with dichloromethane (DCM). The aqueous layer was neutralized with 1.0 N HCl (3.0 mL), and extractd with DCM. 1 N HCl (0.5 mL) was added and the mixture was extracted with DCM (pH˜5.5 after extraction). The procedures were repeated two more time. The last 4 DCM extracts were combined, dried (MgSO₄), filtered, and concentrated to give the desired product (439 mg, 97.6%) as white solid.

### Step4: N-[(3S)-1-(4-bromo-2-fluorophenyl)piperidin-3-yl]-N′-(cis-4-hydroxycyclohexyl)-malonamide

N-(3-dimethylaminopropyl)-N′-ethylcarbodiimide hydrochloride (267 mg, 0.00139 mol) was added to a mixture of 3-[(3S)-1-(4-bromo-2-fluorophenyl)piperidin-3-yl]amino-3-oxopropanoic acid (0.439 g, 0.00122 mol), cis-4-aminocyclohexanol (153 mg, 0.00133 mol), 1-hydroxybenzotriazole (184 mg, 0.00136 mol), and triethylamine (0.88 mL, 0.0063 mol) in methylene chloride (10 mL, 0.2 mol). The reaction was stirred at room temperature for about 15 hours. The mixture was then diluted with DCM, washed with water, and washed with brine. The organic layer was dried over MgSO₄, filtered, and concentrated. The residue was purified on a silica gel column with 0-10% MeOH/DCM to give desired product (359 mg, 64.37%). LCMS: (M+H)+=456.

### Step5: cis-4-[(3-[(3S)-1-(4-bromo-2-fluorophenyl)piperidin-3-yl]aminopropyl)-amino]cyclohexanol

1.0 M of borane in tetrahydrofuran (5.2 mL) was added to N-[(3S)-1-(4-bromo-2-fluorophenyl)piperidin-3-yl]-N′-(cis-4-hydroxycyclohexyl)malonamide (394 mg, 0.000561 mol) at room temperature. The mixture was stirred at room temperature for 16 hours. Additional 1.0 M of borane in tetrahydrofuran (3.4 mL) was added and the mixture was stirred at room temperature for 28 hours. The mixture was then quenched with MeOH and N,N,N′,N′-tetramethylethylenediamine (0.30 mL, 0.0020 mol) was added. The mixture was stirred at room temperature for 17 hours and then evaporated to dryness. Then 13 mL of 6 N HCl was added to the residue. The mixture was heated to 100° C. for 6 h. After cooling, 6.0 mL 50% (12.5 M) NaOH was added dropwise to the mixture. The resulting mixture was diluted with water and then extracted with Et₂O (3×). The combined organic layers were dried over MgSO₄, filtered, and concentrated under reduced pressure to give 30 6 mg of crude product.

### Step6: 1-[(35)-1-(4-bromo-2-fluorophenyl)piperidin-3-yl]-3-(cis-4-hydroxycyclohexyl)-tetrahydropyrimidin-2(1H)-one

The crude product from Step 5 was dissolved in N,N-dimethylformamide (5.0 mL, 0.064 mol) and triethylamine (160 μL, 0.0011 mol). N,N-carbonyldiimidazole (109 mg, 0.000672 mol) in 3.0 mL DCM was added dropwise and the mixture was stirred at room temperature for 6 h. The reaction mixture was diluted with EtOAc and then washed with water (3×). The organic layer was dried over MgSO₄, filtered, and concentrated under reduced pressure. The residue was purified on prep.-HPLC (pH=2 conditions) to give a TFA salt of the desired product (4 mg). LCMS LCMS: (M+H)+=454.

### Example A

Enzymatic assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1 M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1 M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Compounds having an IC₅₀ value less than about 100 μM according to this assay were considered active. The compound of Example 1 was found to have an IC₅₀ value of less than 5 μM.

### Example B

Cell-Based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/ml recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 100 liM according to this assay were considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula Ia, Ib, Ic, Id, Ie, If or Ig: 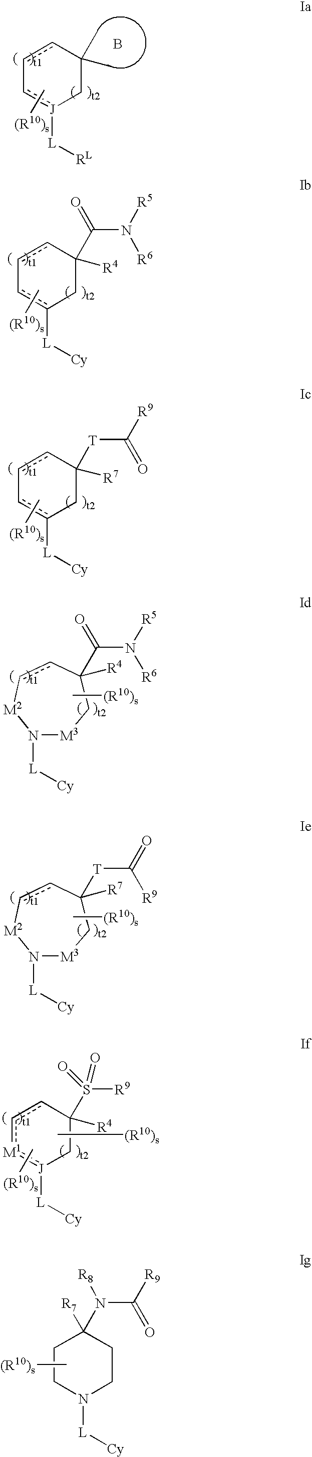
or pharmaceutically acceptable salt or prodrug thereof, wherein:
Cy is aryl, heteroaryl, cycloalkyl, or heterocycloalkyl, each optionally substituted with 1, 2, 3, 4 or 5-W-X—Y-Z;
the ring-forming atom J is N or C;
L is absent, C₁₋₆alkenylenyl, (CR¹R²)_{q}, (CR¹R²)_{q1}O(CR¹R²)_{q2}, (CR¹R²)_{q1}S(CR¹R²)_{q2}, (CR¹R²)_{q1}SO₂(CR¹R²)_{q2}, (CR¹R²)_{q1}SO(CR¹R²)_{q2}, (CR¹R²)_{q1}SO₂NR³(CR¹R²)_{q2}, (CR¹R²)_{q1}COO(CR¹R²)_{q2}, (CR¹R²)_{q1}CO(CR¹R²)_{q2}, (CR¹R²)_{q1}NR^{3a}CONR³(CR¹R²)_{q2}, or (CR¹R²)_{q1}CONR³(CR¹R²)_{q2}, wherein the C₁₋₆ alkenylenyl is optionally substituted by 1, 2, 3, 4, 5 or 6 R^{1a};
M¹ is CH, CH₂, C(O), O, SO, SO₂, OC(O), NH, NHC(O), or NHSO₂;
M² and M³ are independently selected from absent, C(O), SO, SO₂, O, OC(O), NH, NHC(O), and NHSO₂, provided that at least one of M² and M³ is other than absent;
T is NR⁸, CH₂ or O;
ring B is a 3-14 membered cycloalkyl group or 3-14 membered heterocycloalkyl group which is optionally substituted by 1, 2, 3, 4 or 5-W^{a}-X^{a}-W′-X′—Y′-Z′; [symbol]
is a single or double bond;
R^{L} is Cy or C₁₋₆ alkyl wherein the C₁₋₆ alkyl is optionally substituted by 1, 2, 3, 4 or 5-W-X—Y-Z;
R¹ and R² are independently selected from H, halo, C₁₋₆ alkyl, C₁₋₆ haloalkyl, cycloalkyl, heteroaryl, heterocycloalkyl, CN, NO₂, OR^{a′}, SR^{a′}, C(O)R^{b′, C(O)NR}^{c′}R^{d′}, C(O)OR^{a′}, OC(O)R^{b′}, OC(O)NR^{c′}R^{d′}, S(O)R^{b′}, S(O)NR^{c′R}^{d′}, S(O)₂R^{b′}, and S(O)₂NR^{c′}R^{d′};
each R^{1a} is independently selected from halo, C₁₋₆ alkyl, C₁₋₆ haloalkyl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, S(O)R^{b′}, S(O)NR^{c′}R^{d′}, S(O)₂R^{b′}, and S(O)₂NR^{c′}R^{d′};
R³ and R^{3a} are independently selected from H, C₁₋₈ alkyl, arylalkyl, heteroarylalkyl, cycloalkyl, cycloalkylalkyl, heterocycloalkyl and heterocycloalkylalkyl, wherein each of the C₁₋₈ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′;
R⁴ is H, C(O)OR^{b′}, C(O)NR^{c′}R^{d′}, OR^{b′}, SR^{b′}, S(O)R^{a′}, S(O)NR^{c′}R^{d′}, S(O)₂R^{a′}, S(O)₂NR^{c′}R^{d′, C}₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹¹;
R⁵ is H or C₁₋₆ alkyl, wherein the C₁₋₆ alkyl is optionally substituted by 1, 2 or 3-W′-X′′Y′-Z′;
or R⁴ and R⁵ together with the intervening —C—C(O)—N(R⁶)— moiety to which they are attached form a 4-14 membered heterocycloalkyl group which is optionally substituted by 1, 2 or 3-W′-X′—Y′-Z′;
R⁶ is C₁₋₆ alkyl, aryl, cycloalkyl, heteroaryl or heterocycloalkyl, each optionally substituted by 1, 2 or 3-W′-X′—Y′-Z′;
or R⁵ and R⁶ together with the N atom to which they are attached form a 3-14 membered heterocycloalkyl group which is optionally substituted by 1, 2 or 3-W′-X′—Y′-Z′;
R⁷ is H, C(O)OR^{b′}, C(O)NR^{c′}R^{d′}, OR^{b′}, SR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹¹;
R⁸ is H, C₁₋₆ alkyl, arylalkyl, heteroarylalkyl, cycloalkyl, cycloalkylalkyl, heterocycloalkyl or heterocycloalkylalkyl, wherein each of the C₁₋₆ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′;
or R⁷ and R⁸ together with the two adjacent atoms to which they are attached form a 3-14 membered heterocycloalkyl group which is optionally substituted by 1, 2 or 3-W′-X′—Y′-Z′;
R⁹ is H, C₁₋₆ alkyl, arylalkyl, heteroarylalkyl, cycloalkyl, cycloalkylalkyl, heterocycloalkyl or heterocycloalkylalkyl, wherein each of the C₁₋₆ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′;
or R⁸ and R⁹ together with the two adjacent atoms to which they are attached form a 3-14 membered heterocycloalkyl group which is optionally substituted by 1, 2 or 3-W′-X′—Y′-Z′;
or R⁷ and R⁹ together with the intervening —C-T-C(O)— moiety to which they are attached form a 4-14 membered heterocycloalkyl group which is optionally substituted by 1, 2 or 3-W′-X′—Y′-Z′;
or R⁴ and R⁹ together with the intervening —C—S(O)₂— moiety to which they are attached form a 4-14 membered heterocycloalkyl group which is optionally substituted by 1, 2 or 3-W′-X′—Y′-Z′;
or R⁹ is NR^{9a}R^{9b};
R^{9a} and R^{9b} are each, independently, H, C₁₋₆ alkyl, aryl, cycloalkyl, heteroaryl, heterocycloalkyl, each optionally substituted by 1, 2 or 3-W′-X′—Y′-Z′;
or R^{9a} and R^{9b} together with the N atom to which they are attached form a 3-14 membered heterocycloalkyl group which is optionally substituted by 1, 2 or 3-W′-X′—Y′-Z′;
each R¹⁰ is independently OC(O)R^{a′}, OC(O)OR^{b′}, OC(O)NR^{c′}R^{d′}, C(O)OR^{b′}, C(O)NR^{c′}R^{d′}, NR^{c′}R^{d′}, NR^{c′}C(O)R^{a′}, NR^{c′}C(O)OR^{b′}, NR^{c′}S(O)₂R^{b′}, S(O)R^{a′}, S(O)NR^{c′}R^{d′}, S(O)₂R^{a′}, S(O)₂NR^{c′}R^{d′}, OR^{b′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 R¹¹;
or two R¹⁰ together with the same carbon atom to which they are attached form a 3-14 membered cycloalkyl or heterocycloalkyl group which is optionally substituted by 1, 2 or 3 R¹¹;
or two R¹⁰ together with the same carbon atom to which they are attached form a carbonyl group;
or two adjacent R¹⁰ together with the two atoms to which they are attached form a 3-14 membered fused cycloalkyl group or 3-14 membered fused heterocycloalkyl group which is optionally substituted by R¹¹;
or R¹⁰ and -L-Cy together with the same carbon atom to which they are attached form a 3-14 membered cycloalkyl or heterocycloalkyl group which is optionally substituted by 1, 2 or 3 R¹¹;
or adjacent R¹⁰ and -L-Cy together with the two atoms to which they are attached form a 3-14 membered fused cycloalkyl group or 3-14 membered fused heterocycloalkyl group which is optionally substituted by R¹¹;
or R¹⁰ and -L-R^{L} together with the same carbon atom to which they are attached form a 3-14 membered cycloalkyl or heterocycloalkyl group which is optionally substituted by 1, 2 or 3 R¹¹;
or adjacent R¹⁰ and -L-R^{L} together with the two atoms to which they are attached form a 3-14 membered fused cycloalkyl group or 3-14 membered fused heterocycloalkyl group which is optionally substituted by R¹¹;
or adjacent R⁴ and R¹⁰ together with the two atoms to which they are attached form a 3-14 membered fused cycloalkyl group or 3-14 membered fused heterocycloalkyl group which is optionally substituted by R¹⁰;
or adjacent R⁷ and R¹⁰ together with the two atoms to which they are attached form a 3-14 membered fused cycloalkyl group or 3-14 membered fused heterocycloalkyl group which is optionally substituted by R¹¹;
or adjacent R⁴ and -L-Cy together with the two atoms to which they are attached form a 3-14 membered fused cycloalkyl group or 3-14 membered fused heterocycloalkyl group which is optionally substituted by R¹¹;
or adjacent R⁷ and -L-Cy together with the two atoms to which they are attached form a 3-14 membered fused cycloalkyl group or 3-14 membered fused heterocycloalkyl group which is optionally substituted by R¹¹;
or adjacent R⁴ and -L-R^{L} together with the two atoms to which they are attached form a 3-14 membered fused cycloalkyl group or 3-14 membered fused heterocycloalkyl group which is optionally substituted by R¹¹;
or adjacent R⁷ and -L-R^{L} together with the two atoms to which they are attached form a 3-14 membered fused cycloalkyl group or 3-14 membered fused heterocycloalkyl group which is optionally substituted by R¹¹;
each R¹¹ is independently halo, C₁₋₆ alkyl, C₁₋₆ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
W, W′, W″ and W^{a} are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e} and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
X, X, X′ and X^{a} are independently selected from absent, C₁₋₆ alkylenyl, C₂₋₆ alkenylenyl, C₂₋₆ alkynylenyl, aryl, cycloalkyl, heteroaryl and heterocycloalkyl, wherein each of the C₁₋₆ alkylenyl, C₂₋₆ alkenylenyl, C₂₋₆ alkynylenyl, cycloalkyl, heteroaryl and heterocycloalkyl is optionally substituted by 1, 2 or 3 substituents independently selected from halo, CN, NO₂, OH, C₁₋₆ alkyl, C₁₋₆ haloalkyl, C₂₋₈ alkoxyalkyl, C₁₋₆ alkoxy, C₁₋₆ haloalkoxy, C₂₋₈ alkoxyalkoxy, cycloalkyl, heterocycloalkyl, C(O)OR^{a}, C(O)NR^{c}R^{d}, amino, C₁₋₆ alkylamino and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of the C₁₋₆ alkylenyl, C₂₋₆ alkenylenyl and C₂₋₆ alkynylenyl is optionally substituted by 1, 2 or 3 substituents independently selected from halo, OH, C₁₋₆ alkoxy, C₁₋₆ haloalkoxy, amino, C₁₋₆ alkylamino and C₂₋₈ dialkylamino;
Z, Z′ and Z″ are independently selected from H, halo, CN, NO₂, OH, C₁₋₆ alkoxy, C₁₋₆ haloalkoxy, amino, C₁₋₆ alkylamino, C₂₋₈ dialkylamino, C₁₋₆ alkyl, C₂₋₆ alkenyl, C₂₋₆ alkynyl, aryl, cycloalkyl, heteroaryl and heterocycloalkyl, wherein each of the C₁₋₆ alkyl, C₂₋₆ alkenyl, C₂₋₆ alkynyl, aryl, cycloalkyl, heteroaryl and heterocycloalkyl is optionally substituted by 1, 2 or 3 substituents independently selected from halo, C₁₋₆ alkyl, C₂₋₆ alkenyl, C₂₋₆ alkynyl, C₁₋₆ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};
wherein two -W-X—Y-Z attached to the same atom optionally form a 3-14 membered cycloalkylk or 3-14 membered heterocycloalkyl group optionally substituted by 1, 2 or 3-W″-X″—Y″-Z″;
wherein two -W′-X′—Y′-Z′ attached to the same atom optionally form a 3-14 membered cycloalkyl or 3-14 membered heterocycloalkyl group optionally substituted by 1, 2 or 3-W″-X″—Y″-Z″;
wherein two -W^{a}-X^{a}-W′-X′—Y′-Z′ attached to the same atom optionally form a 3-14 membered cycloalkyl or 3-14 membered heterocycloalkyl group optionally substituted by 1, 2 or 3-W″-X″—Y″-Z″;
wherein -W-X—Y-Z is other than H;
wherein -W′-X′—Y′-Z′ is other than H;
wherein -W^{a}-X^{a}-W′-X″—Y-Z, is other than H;
wherein -W″-X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl and heterocycloalkyl, wherein each of the C₁₋₆ alkyl, C₁₋₆ haloalkyl, C₂₋₆ alkenyl, C₂₋₆ alkynyl, aryl, cycloalkyl, heteroaryl and heterocycloalkyl is optionally substituted by OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group;
q is 1, 2or 3;
q1 is 0, 1 or 2;
q2 is 0, 1 or 2;
s is 0, 1, 2, 3, 4, 5, 6, 7, 8, 9, 10 or 11;
t1 is is 0, 1 or 2; and
t2 is 0, 1 or 2;
provided that when the compound has formula Ia and the ring-forming atom J is N, then ring B is other than a ring having the structure: 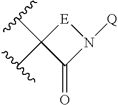
wherein:
Q is —(CR¹⁰¹R¹⁰²)ₘ—R²⁰⁰;
R²⁰⁰ is cycloalkyl, heterocycloalkyl or heteroaryl, each optionally substituted with 1, 2, 3, 4 or 5-W′-X′—Y′-Z′;
E is —(CR^{103a}R^{103b})ₙ₁—, —(CR^{103a}R^{103b})ₙ₂CO—, —(CR^{103a}R^{103b})ₙ₂OCO—, —(CR^{103a}R^{103b})ₙ₂SO—, —(CR^{103a}R^{103b})ₙ₂SO₂—, —(CR^{103a}R^{103b})ₙ₂NR^{103c}—, —(CR^{103a}R^{103b})ₙ₂CONR^{103c}—, —(CR^{103a}R^{3b})ₙ₂NR^{103c}CO—, or a group of formula: 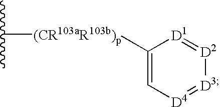
D¹, D², D³ and D⁴ are independently selected from N and CR¹⁰⁴;
R¹⁰¹ and R¹⁰² are independently selected from H and C₁₋₈ alkyl;
R^{103a} and R^{103b} are independently selected from H, halo, C₁₋₄ alkyl, C₁₋₄ haloalkyl, C₂₋₄ alkenyl, and C₂₋₄ alkynyl;
R^{103c} is H, C₁₋₄ alkyl, C₁₋₄ haloalkyl, C₂₋₄ alkenyl, C₂₋₄ alkynyl, or CO—(C₁₋₄ alkyl);
each R¹⁰⁴ is independently H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′, NR}^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
m is 0, 1, 2 or 3;
n1 is 1, 2, 3 or 4;
n2is 0, 1, 2, 3 or 4; and
p is 0, 1 or 2.

2. The compound of claim 1, or pharmaceutically acceptable salt thereof, wherein Cy is aryl or heteroaryl, each optionally substituted with 1, 2, 3, 4 or 5-W-X—Y-Z.

3. The compound of claim 1, or pharmaceutically acceptable salt thereof, wherein Cy is aryl or heteroaryl, each optionally substituted with 1, 2, 3, 4 or 5-W-X—Y-Z wherein W is O or absent, X is absent, and Y is absent.

4. The compound of claim 1, or pharmaceutically acceptable salt thereof, wherein Cy is phenyl, naphthyl, pyridyl, pyrimidinyl, triazinyl, furanyl, thiazolyl, pyrazinyl, purinyl, quinazolinyl, quinolinyl, isoquinolinyl, pyrrolo[2,3-d]pyrimidinyl, or 1,3-benzothiazolyl, each optionally substituted with 1, 2, 3, 4 or 5-W-X—Y-Z.

5. The compound of claim 1, or pharmaceutically acceptable salt thereof, wherein Cy is phenyl, naphthyl, pyridyl, pyrimidinyl, triazinyl, furanyl thiazolyl, pyrazinyl, purinyl, quinazolinyl, quinolinyl, isoquinolinyl, pyrrolo[2,3-d]pyrimidinyl, or 1,3-benzothiazolyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, CN, NO₂, OR^{a}, SR^{a}, C(O)NR^{c}R^{d}, NR^{c}C(O)R^{d} and COOR^{a}.

6. The compound of claim 1, or pharmaceutically acceptable salt thereof, wherein Cy is phenyl, pyridyl, pyrimidinyl, pyrazinyl or 1,3-benzothiazolyl, each optionally substituted by 1, 2, 3, 4 or 5-W-X—Y-Z.

7. The compound of claim 1, or pharmaceutically acceptable salt thereof, wherein Cy is phenyl, pyridyl, pyrimidinyl, pyrazinyl or 1,3-benzothiazolyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, CN, NO₂, OR^{a}, SR^{a}, C(O)NR^{c}R^{d}, NR^{c}C(O)R^{d} and COOR^{a}.

8. The compound of claim 1, or pharmaceutically acceptable salt thereof, wherein Cy is phenyl, pyridyl, pyrimidinyl, pyrazinyl or 1,3-benzothiazolyl, each optionally substituted by 1, 2, 3 or 4 substituents independently selected from halo, CN, C₁₋₆ haloalkyl, C₁₋₆ alkyl and aryl, wherein each of the C₁₋₆ alkyl and aryl is optionally substituted by 1, 2 or 3 substituents independently selected from halo, C₁₋₆ alkyl, C₁₋₆ haloalkyl and CN.

9. The compound of claim 1, or pharmaceutically acceptable salt thereof, wherein Cy is cycloalkyl or heterocycloalkyl, each optionally substituted by 1, 2, 3, 4 or 5-W-X—Y-Z.

10. The compound of claim 1, or pharmaceutically acceptable salt thereof, wherein Cy is cycloalkyl or heterocycloalkyl, each optionally substituted by 1, 2, 3, 4 or 5-W-X—Y-Z wherein W is O or absent, X is absent, and Y is absent.

11. The compound of claim 1, or pharmaceutically acceptable salt thereof, wherein Cy is cyclopropyl, cyclobutyl, cyclopentyl, cyclohexyl, cyclocheptyl, adamantyl, aziridinyl, azetidinyl, pyrrolidine, piperidinyl, 2-oxo-hexahydropyrimidinyl, piperizinyl or morpholinyl, each optionally substituted by 1, 2, 3, 4 or 5-W-X—Y-Z.

12. The compound of claim 1, or pharmaceutically acceptable salt thereof, wherein Cy is cyclopropyl, cyclobutyl, cyclopentyl, cyclohexyl, cyclocheptyl, adamantyl, aziridinyl, azetidinyl, pyrrolidine, piperidinyl, 2-oxo-hexahydropyrimidinyl, piperizinyl or morpholinyl, each optionally substituted by 1, 2, 3 or 4 substituents independently selected from halo, CN, NO₂, C₁₋₆ alkoxy, heteroaryloxy, C₂₋₆ alkynyl, C₁₋₆ haloalkoxy, NR^{c}C(O)R^{d}, NR^{c}C(O)OR^{a}, C(O)NR^{c}R^{d}, NR^{c}R^{d}, NR^{e}S(O)₂R^{b}, C₁₋₆ haloalkyl, C₁₋₆ alkyl, heterocycloalkyl, aryl and heteroaryl, wherein each of the C₁₋₆ alkyl, aryl and heteroaryl is optionally substituted by 1, 2 or 3 substituents independently selected from halo, C₁₋₆ alkyl, C₁₋₆ haloalkyl, CN, NO₂, OR^{a}, SR^{a}, C(O)NR^{c}R^{d}, NR^{c}C(O)R^{d} and COOR^{a}.

13. The compound of claim 1, or pharmaceutically acceptable salt thereof, wherein Cy is cyclohexyl or piperidinyl each optionally substituted by 1, 2, 3, 4 or 5-W-X—Y-Z.

14. The compound of claim 1, or pharmaceutically acceptable salt thereof, wherein L is absent.

15. The compound of claim 1, or pharmaceutically acceptable salt thereof, wherein L is (CR¹R²)_{q1}S(CR¹R²)_{q2}, (CR¹R²)_{q1}SO₂(CR¹R²)_{q2}, (CR¹R²)_{q1}SO(CR¹R²)_{q2} or (CR¹R²)_{q1}SO₂NR³(CR^{1R}²)_{q2}.

16. The compound of claim 1, or pharmaceutically acceptable salt thereof, wherein L is (CR¹R²)_{q1}S(CR¹R²)_{q2} or (CR¹R²)_{q1}SO₂(CR¹R²)_{q2}.

17. The compound of claim 1, or pharmaceutically acceptable salt thereof, wherein L is S, SO, SO₂ or SO₂NH.

18. The compound of claim 1, or pharmaceutically acceptable salt thereof, wherein L is SO₂.

19. The compound of claim 1, or pharmaceutically acceptable salt thereof, wherein L is (CR¹R²)_{q1}COO(CR¹R²)_{q2}, (CR¹R²)_{q1}CO(CR¹R²)_{q2}, (CR¹R²)_{q1}NR^{3a}CONR³(CR¹R²)_{q2}, or (CR¹R²)_{q1}CONR³(CR¹R²)_{q2}.

20. The compound of claim 1, or pharmaceutically acceptable salt thereof, wherein L is COO, CO, COO—C₁₋₃ alkylene, NR^{3a}CONR³ or CONR³.

21. The compound of claim 1, or pharmaceutically acceptable salt thereof, wherein L is (CR¹R²)_{q1}O(CR¹R²)_{q2}.

22. The compound of claim 1, or pharmaceutically acceptable salt thereof, wherein L is O.

23. The compound of claim 1, or pharmaceutically acceptable salt thereof, wherein L is (CR¹R²)_{q}.

24. The compound of claim 1, or pharmaceutically acceptable salt thereof, wherein L is C₁₋₃ alkylene.

25. The compound of claim 1, or pharmaceutically acceptable salt thereof, wherein t1 is 0.

26. The compound of claim 1, or pharmaceutically acceptable salt thereof, wherein t1 is 1 or 2.

27. The compound of claim 1, or pharmaceutically acceptable salt thereof, wherein t2 is 0.

28. The compound of claim 1, or pharmaceutically acceptable salt thereof, wherein t2 is 1 or 2.

29. The compound of claim 1, or pharmaceutically acceptable salt thereof, wherein M¹ is CH or CH₂.

30. The compound of claim 1, or pharmaceutically acceptable salt thereof, wherein M¹ is C(O), O, SO₂, OC(O), NH, NHC(O), or NHSO₂.

31. The compound of claim 1, or pharmaceutically acceptable salt thereof, wherein M² and M³ are independently selected from absent, C(O), OC(O), O, NH, and SO₂.

32. The compound of claim 1, or pharmaceutically acceptable salt thereof, wherein one of M² and M³ is absent, and the other is selected from C(O), OC(O), O, NH, and SO₂.

33. The compound of claim 1, or pharmaceutically acceptable salt thereof, wherein each R¹⁰ is independently OC(O)R^{a′}, OC(O)OR^{b′}, C(O)OR^{b′}, OC(O)NR^{c′}R^{d′}, NR^{c′}R^{d′}, NR^{c}C(O)R^{a′}, NR^{c′}C(O)OR^{b′}, S(O)R^{a′}, S(O)NR^{c′}R^{d′}, S(O)₂R^{a′}, S(O)₂NR^{c}R^{d′}, OR^{b′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

34. The compound of claim 1, or pharmaceutically acceptable salt thereof, wherein each R¹⁰ is independently C(O)OR^{b′}, C₁₋₁₀ alkyl or C₁₋₁₀ haloalkyl.

35. The compound of claim 1, or pharmaceutically acceptable salt thereof, wherein s is 0, 1, 2, or 3.

36. The compound of claim 1, or pharmaceutically acceptable salt thereof, wherein s is 0 or 1.

37. The compound of claim 1, or pharmaceutically acceptable salt thereof, wherein s is 0.

38. The compound of claim 1, or pharmaceutically acceptable salt thereof, wherein the compound has formula Ia.

39. The compound of claim 38, or pharmaceutically acceptable salt thereof, wherein the ring-forming atom J is N.

40. The compound of claim 38, or pharmaceutically acceptable salt thereof, wherein the ring-forming atom J is C.

41. The compound of claim 38, or pharmaceutically acceptable salt thereof, wherein:
ring B is selected from: 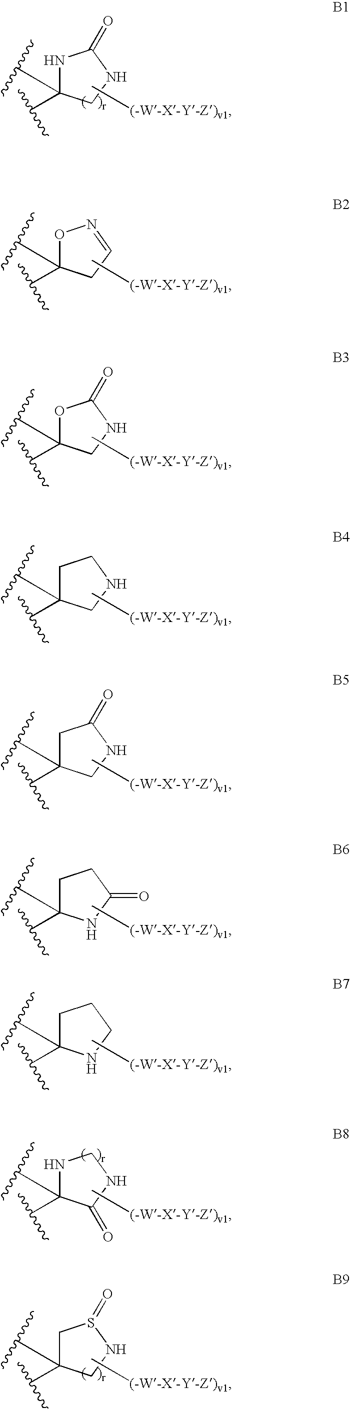 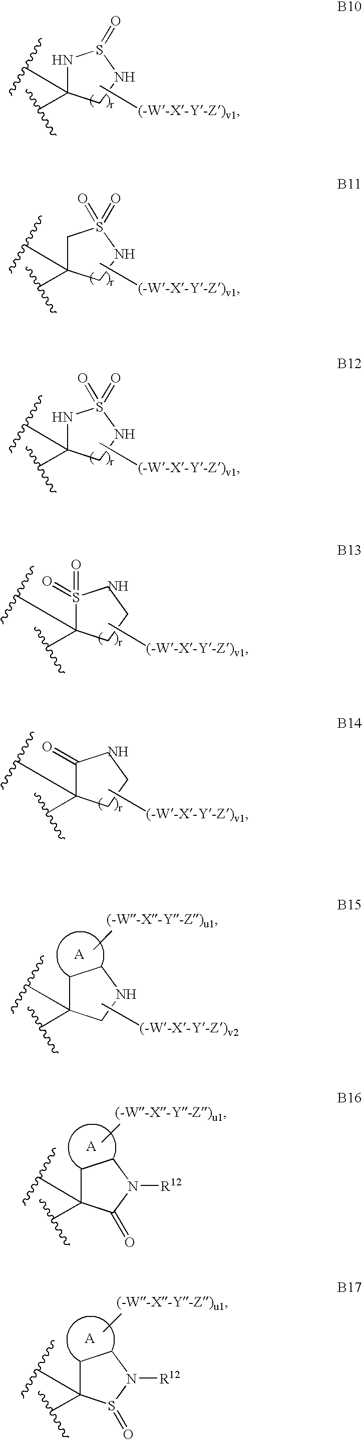 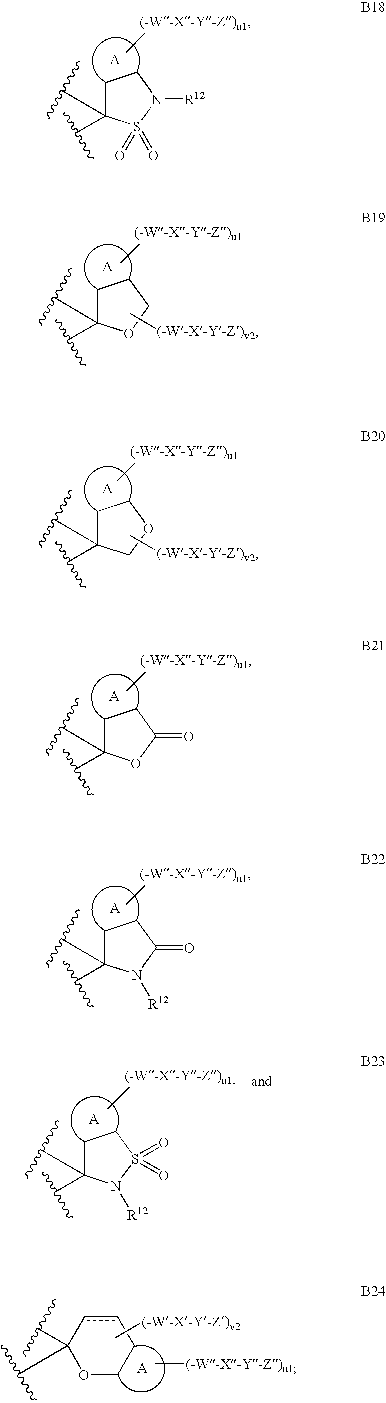 [symbol]
is a single or double bond;
r is 0, 1 or 2;
v1 is 0, 1, 2, or 3;
v2 is 0 or 1;
u1 is 0, 1, 2, or 3;
each R¹² is H or -W′-X′—Y′-Z′; and
ring A is a 5- or 6-membered aryl or heteroaryl.

42. The compound of claim 38, or pharmaceutically acceptable salt thereof, wherein:
ring B is selected from: 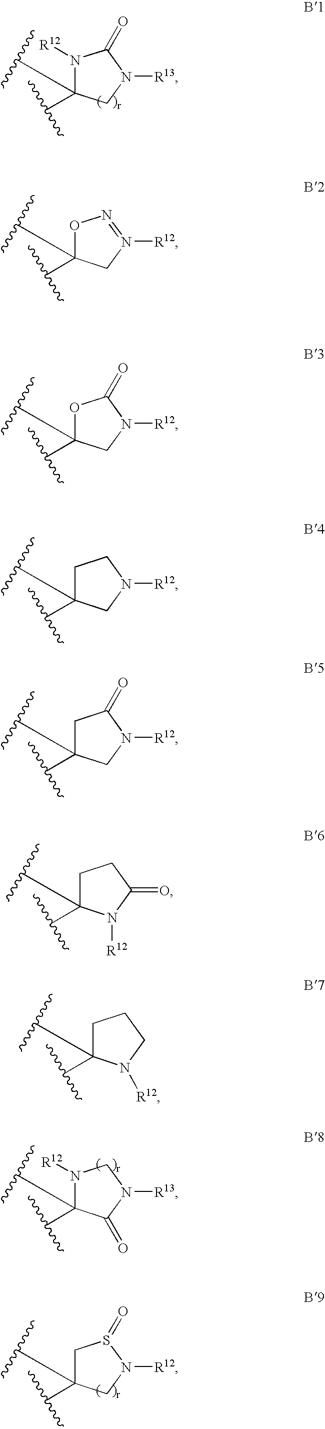 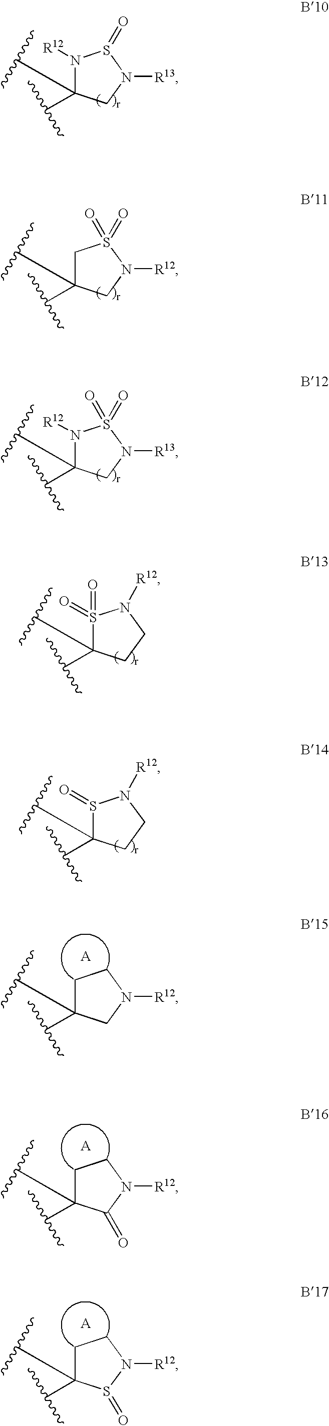 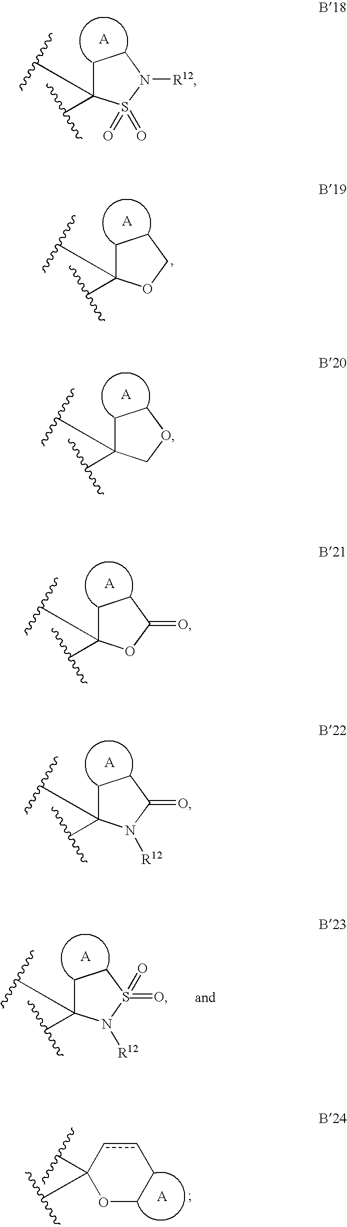
r is 0, 1 or 2;
each R¹² is H or -W′-X′—Y′-Z′;
each R¹³ is H or -W′-X′—Y′-Z′; and
ring A is a 5- or 6-membered aryl or heteroaryl.

43. The compound of claim 42, or pharmaceutically acceptable salt thereof, wherein each of R¹² and R¹³ is independently H, C(O)R^{b}, COOR^{a}, C(O)NR^{c}R^{d}, S(O)₂R^{b}, S(O)₂NR^{c}R^{d}, or C₃₋₁₄ cycloalkyl, wherein the C₃₋₁₄ cycloalkyl is optionally substituted by 1 or 2 substituents independently selected from C₁₋₆ alkyl, C₁₋₆ halolkyl, OH, C₁₋₆ alkoxy, heteroaryloxy, C₁₋₆ haloalkoxy, aryl and heteroaryl, and wherein each of aryl and heteroaryl is optionally substituted by 1 or 2 substituents independently selected from halo, C₁₋₆ alkyl, C₁₋₆ halolkyl and C₁₋₆ haloalkoxy.

44. The compound of claim 38, or pharmaceutically acceptable salt thereof, wherein R^{L} is Cy.

45. The compound of claim 38, or pharmaceutically acceptable salt thereof, wherein R^{L} is C₁₋₆ alkyl optionally substituted by 1, 2, 3, 4 or 5-W-X—Y-Z.

46. The compound of claim 38, or pharmaceutically acceptable salt thereof, wherein L is absent, CO, CONH, COO, or SO₂.

47. The compound of claim 38, or pharmaceutically acceptable salt thereof, wherein:
ring B is selected from: 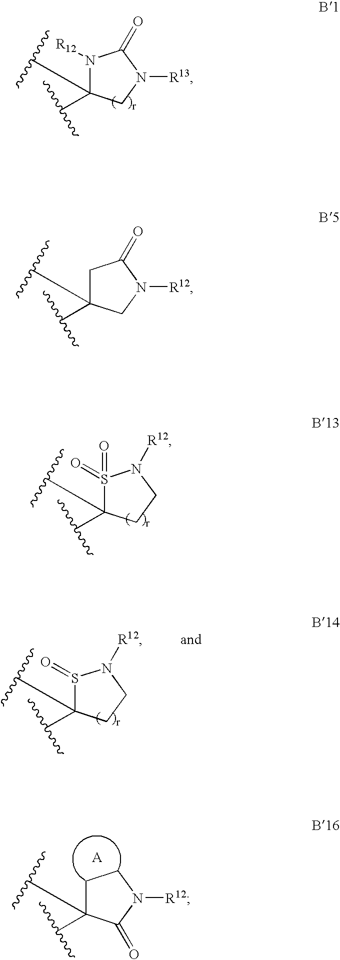
each R¹² is H or -W′-X′—Y′-Z′;
R¹³ is H or -W′-X′—Y′-Z′;
r is 0, 1, or 2;
the ring-forming atom J is C;
L is absent, O or S;
t1 is 0; and
t2 is 1 or 2.

48. The compound of claim 38, or pharmaceutically acceptable salt thereof, wherein:
ring B is selected from: 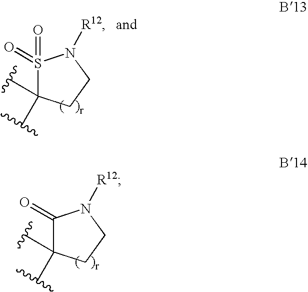
the ring-forming atom J is C;
R¹² is H or -W′-X′—Y′-Z′;
L is absent, O or S;
t1 is 0; and
t2 is 2.

49. The compound of claim 38, or pharmaceutically acceptable salt thereof, wherein:
ring B is selected from: 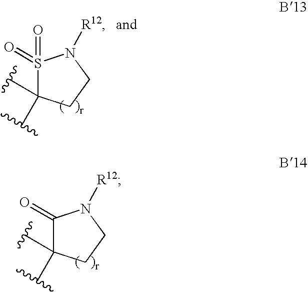
the ring-forming atom J is C;
R¹² is H or -W′-X′—Y′-Z′;
L is absent, O, S or SO₂;
t1 is 1; and
t2 is 1.

50. The compound of claim 38, or pharmaceutically acceptable salt thereof, wherein:
ring B has the structure: 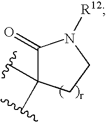
the ring-forming atom J is C;
R¹² is H or -W′-X′—Y′-Z′;
L is absent, CH₂, O, S or SO₂;
t1 is 0; and
t2 is 2.

51. The compound of claim 38, or pharmaceutically acceptable salt thereof, wherein:
ring B has the structure: 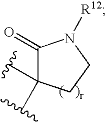
the ring-forming atom J is C;
R¹² is H or -W′-X′—Y′-Z′;
L is absent, CH₂, O, S or SO₂;
t1 is 1; and
t2 is 1.

52. The compound of claim 38, or pharmaceutically acceptable salt thereof, having formula: 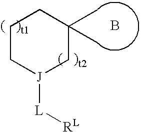
wherein:
t1 is 0 or 1; and
t2 is 1 or 2.

53. The compound of claim 52, or pharmaceutically acceptable salt thereof, wherein:
t1 is 1;
t2 is 1;
L is absent;
R^{L} is Cy.

54. The compound of claim 53, or pharmaceutically acceptable salt thereof, wherein ring B is selected from: 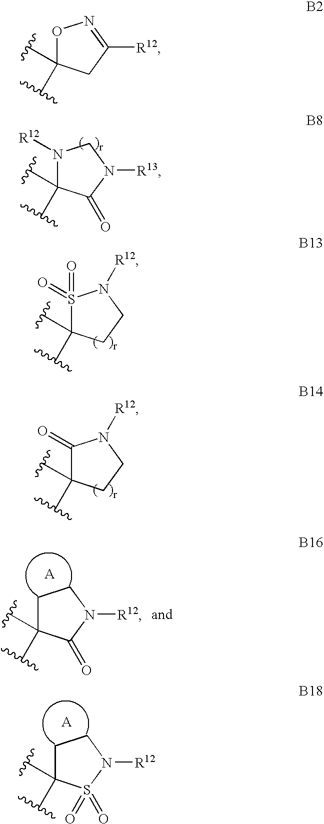
wherein:
r is 0, 1 or 2;
each R¹² is H or -W′-X′—Y′-Z′;
each R¹³ is H or -W′-X′—Y′-Z′; and
ring A is a 5- or 6-membered aryl or heteroaryl.

55. The compound of claim 1, or pharmaceutically acceptable salt thereof, wherein the compound has formula Ib, Id or If.

56. The compound claim 55, or pharmaceutically acceptable salt thereof, wherein R⁴ is H, C(O)OR^{b′} or C₁₋₁₀ alkyl.

57. The compound claim 55, or pharmaceutically acceptable salt thereof, wherein R⁴ is H or C₁₋₁₀ alkyl.

58. The compound of claim 55, or pharmaceutically acceptable salt thereof, wherein R⁵ is cycloalkyl or heterocycloalkyl, each optionally substituted by 1, 2 or 3-W′-X′—Y′-Z′.

59. The compound of claim 55, or pharmaceutically acceptable salt thereof, wherein R⁵ is cycloalkyl optionally substituted by 1, 2 or 3 substituents independently selected from C₁₋₆ alkyl, C₁₋₆ halolkyl, C₁₋₆ hydroxyalkyl, OH, C₁₋₆ alkoxy, C₁₋₆ haloalkoxy, C₂₋₁₂ alkoxyalkoxy, aryloxy and heteroaryloxy.

60. The compound claim 55, or pharmaceutically acceptable salt thereof, wherein R⁶ is H or C₁₋₁₀ alkyl.

61. The compound of claim 55, or pharmaceutically acceptable salt thereof, wherein L is absent, O, C₁₋₃ alkylene, CO, NHCONH, N(C₁₋₄ alkyl)CONH, N(C₁₋₄ alkyl)CON(C₁₋₄ alkyl), CONH, CON(C₁₋₄ alkyl), COO, S, or SO₂.

62. The compound of claim 55, or pharmaceutically acceptable salt thereof, wherein L is absent, O, C₁₋₃ alkylene, CO, CONH, CON(C₁₋₄ alkyl), COO, S, or SO₂.

63. The compound of claim 1, or pharmaceutically acceptable salt thereof, wherein the compound has formula Ib.

64. The compound of claim 63, or pharmaceutically acceptable salt thereof, having the formula:

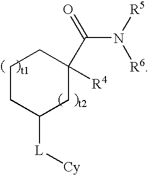

65. The compound of claim 64, or pharmaceutically acceptable salt thereof, wherein:
t1 is 0or 1;
t2 is 1 or 2; and
L is absent, (CR¹R²)_{q}, (CR¹R²)_{q1}O(CR¹R²)_{q2}, (CR¹R²)_{q1}S(CR¹R²)_{q2}, or (CR¹R²)_{q1}SO₂(CR¹R²)_{q2}, (CR¹R²)_{q1}SO(CR¹R²)_{q2}.

66. The compound of claim 65, or pharmaceutically acceptable salt thereof, wherein or R⁴ and R⁵ together with the intervening —C—C(O)—N(R⁶)— moiety to which they are attached form a 4-14 membered heterocycloalkyl group which is optionally substituted by 1, 2 or 3-W′-X′—Y′-Z′.

67. The compound claim 1, or pharmaceutically acceptable salt thereof, wherein:
the compound has formula Ib;
R⁴ is H;
L is absent, CH₂, O, S or SO₂;
R⁵ is cycloalkyl or heterocycloalkyl, each optionally substituted by 1, 2 or 3-W′-X′—Y′-Z′; and
R⁶ is H or C₁₋₆ alkyl.

68. The compound claim 1, or pharmaceutically acceptable salt thereof, wherein:
the compound has formula Ib;
R⁴ is H;
L is absent, CH₂, O, S or SO₂;
R⁵ is cycloalkyl optionally substituted by 1, 2 or 3-W′-X′—Y′-Z′; and
R⁶ is H.

69. The compound claim 1, or pharmaceutically acceptable salt thereof, wherein:
the compound has formula Ib;
R⁴ is H;
L is absent, CH₂, O, S or SO₂;
R⁵ is cycloalkyl optionally substituted by 1, 2 or 3 substituents independently selected from OH and CN; and
R⁶ is H.

70. The compound of claim 1, or pharmaceutically acceptable salt thereof, wherein the compound has formula Ic or Ie.

71. The compound claim 70, or pharmaceutically acceptable salt thereof, wherein R⁷ is H, C(O)OR^{b} or C₁₋₁₀ alkyl.

72. The compound claim 70, or pharmaceutically acceptable salt thereof, wherein R⁹ is H, C₁₋₆ alkyl, arylalkyl, heteroarylalkyl, cycloalkyl, cycloalkylalkyl, heterocycloalkyl or heterocycloalkylalkyl, wherein each of the C₁₋₆ alkyl, arylalkyl, heteroarylalkyl, cycloalkyl, cycloalkylalkyl, heterocycloalkyl and heterocycloalkylalkyl is optionally substituted by 1, 2 or 3-W′-X′—Y′-Z′.

73. The compound claim 70, or pharmaceutically acceptable salt thereof, wherein R⁹ is NR^{9a}R^{9b}; R^{9a} is H or C₁₋₆ alkyl; and R^{9b} is cycloalkyl or heterocycloalkyl, each optionally substituted by 1, 2 or 3-W′-X′—Y′-Z′.

74. The compound claim 70, or pharmaceutically acceptable salt thereof, wherein R⁹ is NR^{9a}R^{9b}; R^{9a} is H or C₁₋₆ alkyl; and R^{9b} is cycloalkyl or heterocycloalkyl, each optionally substituted by 1, 2 or 3 substituents independently selected from C₁₋₆ alkyl, C₁₋₆ halolkyl, C₁₋₆ hydroxyalkyl, OH, C₁₋₆ alkoxy, C₁₋₆ haloalkoxy and C₂₋₈ alkoxyalkoxy.

75. The compound claim 70, or pharmaceutically acceptable salt thereof, wherein T is O or CH₂.

76. The compound claim 70, or pharmaceutically acceptable salt thereof, wherein T is NR⁸; and R⁸ is H or C₁₋₁₀ alkyl.

77. The compound claim 70, or pharmaceutically acceptable salt thereof, wherein T is NR⁸; and R⁸ and R⁹ together with the two adjacent atoms to which they are attached form a 3-14 membered heterocycloalkyl group which is optionally substituted by 1, 2 or 3-W′-X′—Y′-Z′.

78. The compound claim 1, or pharmaceutically acceptable salt thereof, wherein:
the compound has formula Ic;
R⁷ is H;
L is absent, CH₂, O, S or SO₂; and
T is NR⁸.

79. The compound claim 1, or pharmaceutically acceptable salt thereof, wherein:
the compound has formula Ic;
R⁷ is H;
L is absent, CH₂, O, S or SO₂; and
T is NH.

80. The compound claim 1, or pharmaceutically acceptable salt thereof, wherein:
the compound has formula Ic;
R⁷ is H;
L is absent, CH₂, O, S or SO₂;
T is NH; and
R⁹ is NR^{9a}R^{9b}.

81. The compound claim 1, or pharmaceutically acceptable salt thereof, wherein:
the compound has formula Ic;
R⁷ is H;
L is absent, CH₂, O, S or SO₂;
T is NH;
R⁹ is NR^{9a}R^{9b};
R^{9a} is H or C₁₋₆ alkyl; and
R^{9b} is cycloalkyl or heterocycloalkyl, each optionally substituted by 1, 2 or 3-W′-X′—Y′-Z′.

82. The compound claim 1, or pharmaceutically acceptable salt thereof, wherein:
the compound has formula Ic;
R⁷ is H;
L is absent, CH₂, O, S or SO₂;
T is NH; and
R⁸ and R⁹ together with the two adjacent atoms to which they are attached form a 3-14 membered heterocycloalkyl group which is optionally substituted by 1, 2 or 3-W′-X′—Y′-Z′.

83. The compound of claim 1, or pharmaceutically acceptable salt thereof, having formula Ic.

84. The compound of claim 83, or pharmaceutically acceptable salt thereof, having the formula:

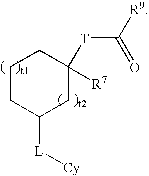

85. The compound of claim 84, or pharmaceutically acceptable salt thereof, wherein:
t1 is 0 or 1;
t2 is 1 or 2;
L is absent, (CR¹R²)_{q}, (CR¹R²)_{q1}O(CR¹R²)_{q2}, (CR¹R²)_{q1}S(CR¹R²)_{q2}, or (CR¹R²)_{q1}SO₂(CR¹R²)_{q2}, or (CR¹R²)_{q1}SO(CR¹R²)_{q2}.

86. The compound of claim 85, or pharmaceutically acceptable salt thereof, wherein R⁸ and R⁹ together with the two adjacent atoms to which they are attached form a 3-14 membered heterocycloalkyl group which is optionally substituted by 1, 2 or 3-W′-X′—Y′-Z′.

87. The compound of claim 1, or pharmaceutically acceptable salt thereof, having formula If.

88. The compound of claim 87, or pharmaceutically acceptable salt thereof, having the formula: 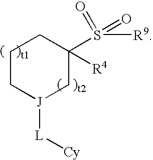
wherein:
t1 is 0 or 1;
t2 is 1 or 2; and
L is absent, (CR¹R²)_{q}, (CR¹R²)_{q1}O(CR¹R²)_{q2}, (CR¹R²)_{q1}S(CR¹R²)_{q2}, or (CR¹R²)_{q1}SO₂(CR¹R²)_{q2}, or (CR¹R²)_{q1}SO(CR¹R²)_{q2}.

89. A compound selected from:
7-Acetyl-3-[1-(4-chlorophenyl)cyclopropyl]-1-oxa-2,7-diazaspiro[4.4]non-2-ene;
Methyl 7-acetyl-3-[1-(4-chlorophenyl)cyclopropyl]-1-oxa-2,7-diazaspiro[4.4]non-2-ene-8-carboxylate;
Methyl 3-[1-(4-chlorophenyl)cyclopropyl]-7-(methylsulfonyl)-1-oxa-2,7-diazaspiro[4.4]non-2-ene-8-carboxylate;
Dimethyl 3-[1-(4-chlorophenyl)cyclopropyl]-1-oxa-2,7-diazaspiro[4.4]non-2-ene-7,8-dicarboxylate;
8-Acetyl-3-[1-(4-chlorophenyl)cyclopropyl]-1-oxa-2,8-diazaspiro[4.5]dec-2-ene;
3-[1-(4-Chlorophenyl)cyclopropyl]-8-(methylsulfonyl)-1-oxa-2,8-diazaspiro[4.5]dec-2-ene;
Methyl 3-cyclohexyl-1-oxa-2,7-diazaspiro[4.5]dec-2-ene-7-carboxylate;
7-[(3-Chloro-2-methylphenyl)sulfonyl]-3-cyclohexyl-1-oxa-2,7-diazaspiro[4.5]dec-2-ene;
3-[1-(4-chlorophenyl)cyclopropyl]-8-phenyl-1-oxa-2-azaspiro[4.5]dec-2-ene;
1′-[(3-chloro-2-methylphenyl)sulfonyl]spiro[indole-3,4′-piperidin]-2(1H)-one;
1′-[(3-chloro-2-methylphenyl)sulfonyl]-3H-spiro[2-benzofuran-1,4′-piperidine];
1′-[(3-chloro-2-methylphenyl)sulfonyl]spiro[chromene-2,4′-piperidine];
8-[(3-chloro-2-methylphenyl)sulfonyl]-2,8-diazaspiro[4.5]decan-3-one;
3-(4-Chlorophenoxy)-N-cyclohexylcyclohexanecarboxamide;
3-(2-Chlorophenoxy)-N-cyclohexylcyclohexanecarboxamide;
N-Cyclohexyl-3-(3-fluorophenoxy)cyclohexanecarboxamide;
N-Cyclohexyl-3-(pyridin-2-yloxy)cyclohexanecarboxamide;
3-[4-Chloro-3-(trifluoromethyl)phenoxy]-N-cyclohexylcyclohexanecarboxamide;
N-Cyclohexyl-3-[(4-fluorophenyl)thio]cyclohexanecarboxamide;
N-Cyclohexyl-3-[(2,4-dichlorophenyl)thio]cyclohexanecarboxamide;
N-Cyclohexyl-3-[(2,4-dichlorophenyl)sulfonyl]cyclohexanecarboxamide;
3-(2-Chlorophenoxy)-N-cyclohexylcyclohexanecarboxamide;
N-Cyclohexyl-3-(pyridin-2-yloxy)cyclohexanecarboxamide;
1′-[4-(4-chlorophenyl)cyclohexyl]carbonyl-3H-spiro[2-benzofuran-1,3′-pyrrolidin]-3-one;
N-cyclohexyl-3-(4-methoxyphenyl)cyclohex-2-ene-1-carboxamide;
N-1-adamantyl-3-oxo-1′H,3H-spiro[2-benzofuran-1,4′-piperidine]-1′-carboxamide;
N-1-adamantyl-1′H,3H-spiro[2-benzofuran-1,4′-piperidine]-1′-carboxamide;
N-1-adamantyl-2-oxo-1,2-dihydro-1′H-spiro[indole-3,4′-piperidine]-1′-carboxamide;
N-1-adamantyl-4-oxo-1-phenyl-1,3,8-triazaspiro[4.5]decane-8-carboxamide;
3-(4-Ethoxybenzyl)-N-(trans-4-hydroxycyclohexyl)cyclohexanecarboxamide;
4-(4-Bromo-2-methylphenyl)-4-hydroxy-N-(4-hydroxycyclohexyl)cyclohexanecarboxamide;
4-(4-Bromo-2-methylphenyl)-N-(4-hydroxycyclohexyl)cyclohex-3-ene-1-carboxamide;
5-[4-(1-Hydroxy-4-{[(4-hydroxycyclohexyl)amino]carbonyl}cyclohexyl)-3-methylphenyl]-N-methylpyridine-2-carboxamide;
4′(1-Hydroxy-4-{[(4-hydroxycyclohexyl)amino]carbonyl}cyclohexyl)-N,3′-dimethylbiphenyl-4-carboxamide;
1′-(3-Chloropyridin-2-yl)spiro[indole-3,4′-piperidin]-2(1H)-one;
4′-(4-[(cis-4-hydroxycyclohexyl)amino]carbonylcyclohex-1-en-1-yl)-N,3′-dimethylbiphenyl-4-carboxamide;
4′-(4-{[(cis-4-hydroxycyclohexyl)amino]carbonyl}cyclohexyl)-N,3′-dimethylbiphenyl-4-carboxamide;
5-[4-(4-[(cis-4-hydroxycyclohexyl)amino]carbonylcyclohex-1-en-1-yl)-3-methylphenyl]-N-methylpyridine-2-carboxamide;
N-(cis-4-hydroxycyclohexyl)-4-(2-methylphenyl)cyclohex-3-ene-1-carboxamide;
N-(cis-4-hydroxycyclohexyl)-4-(2-methylphenyl)cyclohexanecarboxamide;
1′-(piperidin-1-ylcarbonyl)-3H-spiro[2-benzofuran-1,4′-piperidin]-3-one;
1′-(piperidin-1-ylcarbonyl)-3H-spiro[2-benzofuran-1,4′-piperidine];
1′-(piperidin-1-ylcarbonyl)spiro[indole-3,4′-piperidin]-2(1H)-one;
8-(piperidin-1-ylcarbonyl)-1-phenyl-1,3,8-triazaspiro[4.5]decan-4-one;
7-(5-bromo-3-chloropyridin-2-yl)-2-(cyclohexylcarbonyl)-2,7-diazaspiro[4.5]decane;
7-(5-bromo-3-chloropyridin-2-yl)-N-cyclohexyl-2,7-diazaspiro[4.5]decane-2-carboxamide;
7-(5-bromo-3-chloropyridin-2-yl)-2-(piperidin-1-ylcarbonyl)-2,7-diazaspiro[4.5]decane;
7-(5-bromo-3-chloropyridin-2-yl)-2-(morpholin-4-ylcarbonyl)-2,7-diazaspiro[4.5]decane;
7-(5-bromo-3-chloropyridin-2-yl)-2-(3-methoxybenzoyl)-2,7-diazaspiro[4.5]decane;
7-(5-bromo-3-chloropyridin-2-yl)-2-(2-chlorobenzoyl)-2,7-diazaspiro[4.5]decane;
2-(1-adamantylcarbonyl)-7-(5-bromo-3-chloropyridin-2-yl)-2,7-diazaspiro[4.5]decane; and
N-1-adamantyl-7-(5-bromo-3-chloropyridin-2-yl)-2,7-diazaspiro[4.5]decane-2-carboxamide,
or a pharmaceutically acceptable salt thereof.

90. A compound selected from:
8-(4-Chlorophenyl)-2-(trans-4-hydroxycyclohexyl)-2-azaspiro[4.5]decan-1-one;
8-(4-Bromophenoxy)-2-(trans-4-hydroxycyclohexyl)-2-azaspiro[4.5]decan-1-one;
3-(cis-4-Hydroxycyclohexyl)-1-methyl-8-phenyl-1,3-diazaspiro[4.5]decane-2,4-dione;
N-[1-(4-Cyanophenyl)-4-methylpiperidin-4-yl]-2-oxa-6-azatricyclo[3.3.1.1(3,7)]decane-6-carboxamide;
N-4-methyl-1-[4-(trifluoromethyl)phenyl]piperidin-4-yl-2-oxa-6-azatricyclo[3.3.1.1(3,7)]decane-6-carboxamide;
N-[1-(2-fluoro-4-methylphenyl)-4-methylpiperidin-4-yl]-2-oxa-6-azatricyclo[3.3.1.1(3,7)]decane-6-carboxamide;
N-[1-(2-Chlorophenyl)-4-methylpiperidin-4-yl]-2-oxa-6-azatricyclo[3.3.1.1(3,7)]decane-6-carboxamide;
N-[1-(2,3-difluorophenyl)-4-methylpiperidin-4-yl]-2-oxa-6-azatricyclo[3.3.1.1(3,7)]decane-6-carboxamide; and
1-[(3S)-1-(4-Bromo-2-fluorophenyl)piperidin-3-yl]-3-(cis-4-hydroxycyclohexyl)-tetrahydropyrimidin-2(1H)-one,
or pharmaceutically acceptable salt thereof.

91. A composition comprising a compound of claim 1, or pharmaceutically acceptable salt thereof, and at least one pharmaceutically acceptable carrier.

92. A method of modulating 11βHSD1 comprising contacting said 11βHSD1 with a compound of claim 1, or pharmaceutically acceptable salt thereof.

93. The method of claim 92 wherein said modulating is inhibiting.

94. A method of treating a disease in a patient, wherein said disease is associated with expression or activity of 11βHSD1, comprising administering to said patient a therapeutically effective amount of a compound of claim 1, or pharmaceutically acceptable salt thereof.

95. The method of claim 94, or pharmaceutically acceptable salt thereof, wherein said disease is obesity, diabetes, glucose intolerance, insulin resistance, hyperglycemia, atherosclerosis, hypertension, hyperlipidemia, cognitive impairment, dementia, depression, glaucoma, cardiovascular disorders, osteoporosis, inflammation, metabolic syndrome, coronary heart disease, type 2 diabetes, hypercortisolemia, androgen excess, or polycystic ovary syndrome (PCOS).

96. A method of treating metabolic syndrome in a patient comprising administering to said patient a therapeutically effective amount of a compound of claim 1, or pharmaceutically acceptable salt thereof.

97. A method of treating type 2 diabetes in a patient comprising administering to said patient a therapeutically effective amount of a compound of claim 1, or pharmaceutically acceptable salt thereof.
